SEQUENCE LISTING (34 sequences)

SEQ ID NO: 1 - protein, 1255 aa, SARS-CoV
MFIFLLFLTLTSGSDLDRCTTFDDVQAPNYTQHTSSMRGVYYPDEIFRSDTLYLTQDLFLPFYSNVTGFHTINHTFDNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDTWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 2 - protein, 76 aa, SARS-CoV
MYSFVSEETGTLIVNSVLLFLAFVVFLLVTLAILTALRLCAYCCNIVNVSLVKPTVYVYSRVKNLNSSEGVPDLLV

SEQ ID NO: 3 - protein, 221 aa, SARS-CoV
MADNGTITVEELKQLLEQWNLVIGFLFLAWIMLLQFAYSNRNRFLYIIKLVFLWLLWPVTLACFVLAAVYRINWVTGGIAIAMACIVGLMWLSYFVASFRLFARTRSMWSFNPETNILLNVPLRGTIVTRPLMESELVIGAVIIRGHLRMAGHSLGRCDIKDLPKEITVATSRTLSYYKLGASQRVGTDSGFAAYNRYRIGNYKLNTDHAGSNDNIALLVQ

SEQ ID NO: 4 - protein, 422 aa, SARS-CoV
MSDNGPQSNQRSAPRITFGGPTDSTDNNQNGGRNGARPKQRRPQGLPNNTASWFTALTQHGKEELRFPRGQGVPINTNSGPDDQIGYYRRATRRVRGGDGKMKELSPRWYFYYLGTGPEASLPYGANKEGIVWVATEGALNTPKDHIGTRNPNNNAATVLQLPQGTTLPKGFYAEGSRGGSQASSRSSSRSRGNSRNSTPGSSRGNSPARMASGGGETALALLLLDRLNQLESKVSGKGQQQQGQTVTKKSAAEASKKPRQKRTATKQYNVTQAFGRRGPEQTQGNFGDQDLIRQGTDYKHWPQIAQFAPSASAFFGMSRIGMEVTPSGTWLTYHGAIKLDDKDPQFKDNVILLNKHIDAYKTFPPTEPKKDKKKKTDEAQPLPQRQKKQPTVTLLPAADMDDFSRQLQNSMSGASADSTQA

SEQ ID NO: 5 - protein, 1282 aa, SARS-CoV-2
MFLLTTKRTMFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 6 - protein, 75 aa, SARS-CoV-2
MYSFVSEETGTLIVNSVLLFLAFVVFLLVTLAILTALRLCAYCCNIVNVSLVKPSFYVYSRVKNLNSSRVPDLLV

SEQ ID NO: 7 - protein, 222 aa, SARS-CoV-2
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 8 - protein, 419 aa, SARS-CoV-2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 9 - protein, 654 aa, SARS-CoV
SDLDRCTTFDDVQAPNYTQHTSSMRGVYYPDEIFRSDTLYLTQDLFLPFYSNVTGFHTINHTFDNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDTWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLR

SEQ ID NO: 10 - protein, 222 aa, SARS-CoV
RVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNF

SEQ ID NO: 11 - protein, 71 aa, SARS-CoV
NTRNIDATSTGNYNYKYRYLRHGKLRPFERDISNVPFSPDGKPCTPPALNCYWPLNDYGFYTTTGIGYQPY

SEQ ID NO: 12 - protein, 673 aa, SARS-CoV-2
SQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAR

SEQ ID NO: 13 - protein, 223 aa, SARS-CoV-2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 14 - protein, 72 aa, SARS-CoV-2
NSNNLDSKVGGNYNYLYRLFRKSNLKPFERDISTEIYQAGSTPCNGVEGFNCYFPLQSYGFQPTNGVGYQPY

SEQ ID NO: 15 - protein, 805 aa, Homo sapiens [sequence omitted]

SEQ ID NO: 16 - protein, 555 aa, Homo sapiens
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLL

SEQ ID NO: 17 - protein, 723 aa, Homo sapiens
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 18 - protein, 805 aa, Rhinolophus ferrumequinum
MSGSSWFLLSLVAVTAAQSTTEDLAKKFLDDFNSEAENLSHQSSLASWEYNTNISDENVQKMDEAGAKWSDFYEKQSKLAKNFSLEEIHNDTVKLQLQILQQSGSPVLSEDKSKRLNSILNAMSTIYSTGKVCKPNNPQECLLLEPGLDNIMGTSKDYNERLWAWEGWRAEVGKQLRPLYEEYVVLKNEMARGYHYEDYGDYWRRDYETEGSPDLEYSRDQLIKDVERIFAEIKPLYEQLHAYVRTKLMDTYPFHISPTGCLPAHLLGDMWGRFWTNLYPLTVPFGQKPNIDVTDAMLNQNWDAKRIFKEAEKFLVSIGLPNMTEGFWNNSMLTDPGDGRKVVCHPTAWDLGKGDFRIKMCTKVTMEDFLTAHHEMGHIQYDMAYASQPYLLRNGANEGFHEAVGEVMSLSVATPEHLKTMGLLSSDFLEDNETEINFLFKQALNIVGTLPLTYMLEKWRWMVFKGEIPKEEWMKKWWEMKRKIVGVVEPVPHDETYCDPASLFHVANDYSFIRYYTRTIFEFQFHEALCRIAKHDGPLHKCGISNSTDAGEKLHQMLSVGKSQPWTSVLKDFVGSKNMDVGPLLRYFEPLYTWLTEQNRKSFVGWNTDWSPYADQSIKVWISLKSALGEKAYEWNNNEMYLFRSSVAYAMREYFLKTKNQTILFGEEDVWVSNLKPRISFNFYVTSPRNLSDIIPRPEVEGAIRMSRSRINDAFRLDDNSLEFLGIQPTLGPPYQPPVTIWLIVFGVVMAVVVVGIVVLIITGIRDRRKKDQARSEENPYSSVDLSKGENNPGFQNGNDVQTSF

SEQ ID NO: 19 - protein, 723 aa, Rhinolophus ferrumequinum
QSTTEDLAKKFLDDFNSEAENLSHQSSLASWEYNTNISDENVQKMDEAGAKWSDFYEKQSKLAKNFSLEEIHNDTVKLQLQILQQSGSPVLSEDKSKRLNSILNAMSTIYSTGKVCKPNNPQECLLLEPGLDNIMGTSKDYNERLWAWEGWRAEVGKQLRPLYEEYVVLKNEMARGYHYEDYGDYWRRDYETEGSPDLEYSRDQLIKDVERIFAEIKPLYEQLHAYVRTKLMDTYPFHISPTGCLPAHLLGDMWGRFWTNLYPLTVPFGQKPNIDVTDAMLNQNWDAKRIFKEAEKFLVSIGLPNMTEGFWNNSMLTDPGDGRKVVCHPTAWDLGKGDFRIKMCTKVTMEDFLTAHHEMGHIQYDMAYASQPYLLRNGANEGFHEAVGEVMSLSVATPEHLKTMGLLSSDFLEDNETEINFLFKQALNIVGTLPLTYMLEKWRWMVFKGEIPKEEWMKKWWEMKRKIVGVVEPVPHDETYCDPASLFHVANDYSFIRYYTRTIFEFQFHEALCRIAKHDGPLHKCGISNSTDAGEKLHQMLSVGKSQPWTSVLKDFVGSKNMDVGPLLRYFEPLYTWLTEQNRKSFVGWNTDWSPYADQSIKVWISLKSALGEKAYEWNNNEMYLFRSSVAYAMREYFLKTKNQTILFGEEDVWVSNLKPRISFNFYVTSPRNLSDIIPRPEVEGAIRMSRSRINDAFRLDDNSLEFLGIQPTLGPPYQPPVT

SEQ ID NO: 20 - protein, 805 aa, Manis javanica
MSGSSWLLLSLVAVTAAQSTSDEEAKTFLEKFNSEAEELSYQSSLASWNYNTNITDENVQKMNVAGAKWSTFYEEQSKIAKNYQLQNIQNDTIKRQLQALQLSGSSALSADKNQRLNTILNTMSTIYSTGKVCNPGNPQECSLLEPGLDNIMESSKDYNERLWAWEGWRSEVGKQLRPLYEEYVVLKNEMARANHYEDYGDYWRGDYEAEGANGYNYSRDHLIEDVEHIFTQIKPLYEHLHAYVRAKLMDNYPSHISPTGCLPAHLLGDMWGRFWTNLYPLTVPFRQKPNIDVTDAMVNQTWDANRIFKEAEKFFVSVGLPKMTQTFWENSMLTEPGDGRKVVCHPTAWDLGKHDFRIKMCTKVTMDDFLTAHHEMGHIQYDMAYAMQPYLLRNGANEGFHEAVGEIMSLSAATPKHLKNIGLLPPDFYEDNETEINFLLKQALTIVGTLPFTYMLEKWRWMVFSGQIPKEQWMKKWWEMKREIVGVVEPVPHDETYCDPASLFHVANDYSFIRYYTRTIYQFQFQEALCQTAKHEGPLHKCDISNSAEAGQKLLQMLSLGKSKPWTLALERVVGTKNMDVRPLLNYFEPLLTWLKEQNKNSFVGWNTDWSPYAAQSIKVRISLKSALGEKAYEWNDSEMYLFRSSVAYAMREYFSKVKKQTIPFEDECVRVSDLKPRVSFIFFVTLPKNVSAVIPRAEVEEAIRISRSRINDAFRLDDNSLEFLGIQPTLQPPYQPPVTIWLIVFGVVMGVVVVGIVVLIFTGIRDRKKKDQARSEQNPYASVDLSKGENNPGFQNVDDVQTSF

SEQ ID NO: 21 - protein, 723 aa, Manis javanica
QSTSDEEAKTFLEKFNSEAEELSYQSSLASWNYNTNITDENVQKMNVAGAKWSTFYEEQSKIAKNYQLQNIQNDTIKRQLQALQLSGSSALSADKNQRLNTILNTMSTIYSTGKVCNPGNPQECSLLEPGLDNIMESSKDYNERLWAWEGWRSEVGKQLRPLYEEYVVLKNEMARANHYEDYGDYWRGDYEAEGANGYNYSRDHLIEDVEHIFTQIKPLYEHLHAYVRAKLMDNYPSHISPTGCLPAHLLGDMWGRFWTNLYPLTVPFRQKPNIDVTDAMVNQTWDANRIFKEAEKFFVSVGLPKMTQTFWENSMLTEPGDGRKVVCHPTAWDLGKHDFRIKMCTKVTMDDFLTAHHEMGHIQYDMAYAMQPYLLRNGANEGFHEAVGEIMSLSAATPKHLKNIGLLPPDFYEDNETEINFLLKQALTIVGTLPFTYMLEKWRWMVFSGQIPKEQWMKKWWEMKREIVGVVEPVPHDETYCDPASLFHVANDYSFIRYYTRTIYQFQFQEALCQTAKHEGPLHKCDISNSAEAGQKLLQMLSLGKSKPWTLALERVVGTKNMDVRPLLNYFEPLLTWLKEQNKNSFVGWNTDWSPYAAQSIKVRISLKSALGEKAYEWNDSEMYLFRSSVAYAMREYFSKVKKQTIPFEDECVRVSDLKPRVSFIFFVTLPKNVSAVIPRAEVEEAIRISRSRINDAFRLDDNSLEFLGIQPTLQPPYQPPVT

SEQ ID NO: 22 - protein, 805 aa, Paguma larvata
MSGSFWLLLSFAALTAAQSTTEELAKTFLETFNYEAQELSYQSSVASWNYNTNITDENAKNMNEAGAKWSAYYEEQSKLAQTYPLAEIQDAKIKRQLQALQQSGSSVLSADKSQRLNTILNAMSTIYSTGKACNPNNPQECLLLEPGLDNIMENSKDYNERLWAWEGWRAEVGKQLRPLYEEYVALKNEMARANNYEDYGDYWRGDYEEEWTGGYNYSRNQLIQDVEDTFEQIKPLYQHLHAYVRAKLMDTYPSRISRTGCLPAHLLGDMWGRFWTNLYPLTVPFGQKPNIDVTDAMVNQNWDARRIFKEAEKFFVSVGLPNMTQGFWENSMLTEPGDGRKVVCHPTAWDLGKGDFRIKMCTKVTMDDFLTAHHEMGHIQYDMAYAAQPFLLRNGANEGFHEAVGEIMSLSAATPNHLKTIGLLSPAFSEDNETEINFLLKQALTIVGTLPFTYMLEKWRWMVFKGAIPKEQWMQKWWEMKRNIVGVVEPVPHDETYCDPASLFHVANDYSFIRYYTRTIYQFQFQEALCQIAKHEGPLHKCDISNSTEAGKKLLEMLSLGRSEPWTLALERVVGAKNMNVTPLLNYFEPLFTWLKEQNRNSFVGWDTDWRPYSDQSIKVRISLKSALGEKAYEWNDNEMYLFRSSIAYAMREYFSKVKNQTIPFVEDNVWVSDLKPRISFNFFVTFSNNVSDVIPRSEVEDAIRMSRSRINDAFRLDDNSLEFLGIEPTLSPPYRPPVTIWLIVFGVVMGAIVVGIVLLIVSGIRNRRKNDQAGSEENPYASVDLNKGENNPGFQHADDVQTSF

SEQ ID NO: 23 - protein, 723 aa, Paguma larvata
QSTTEELAKTFLETFNYEAQELSYQSSVASWNYNTNITDENAKNMNEAGAKWSAYYEEQSKLAQTYPLAEIQDAKIKRQLQALQQSGSSVLSADKSQRLNTILNAMSTIYSTGKACNPNNPQECLLLEPGLDNIMENSKDYNERLWAWEGWRAEVGKQLRPLYEEYVALKNEMARANNYEDYGDYWRGDYEEEWTGGYNYSRNQLIQDVEDTFEQIKPLYQHLHAYVRAKLMDTYPSRISRTGCLPAHLLGDMWGRFWTNLYPLTVPFGQKPNIDVTDAMVNQNWDARRIFKEAEKFFVSVGLPNMTQGFWENSMLTEPGDGRKVVCHPTAWDLGKGDFRIKMCTKVTMDDFLTAHHEMGHIQYDMAYAAQPFLLRNGANEGFHEAVGEIMSLSAATPNHLKTIGLLSPAFSEDNETEINFLLKQALTIVGTLPFTYMLEKWRWMVFKGAIPKEQWMQKWWEMKRNIVGVVEPVPHDETYCDPASLFHVANDYSFIRYYTRTIYQFQFQEALCQIAKHEGPLHKCDISNSTEAGKKLLEMLSLGRSEPWTLALERVVGAKNMNVTPLLNYFEPLFTWLKEQNRNSFVGWDTDWRPYSDQSIKVRISLKSALGEKAYEWNDNEMYLFRSSIAYAMREYFSKVKNQTIPFVEDNVWVSDLKPRISFNFFVTFSNNVSDVIPRSEVEDAIRMSRSRINDAFRLDDNSLEFLGIEPTLSPPYRPPVT

SEQ ID NO: 24 - protein, 805 aa, Sus scrofa
MSGSFWLLLSLIPVTAAQSTTEELAKTFLEKFNLEAEDLAYQSSLASWNYNTNITDENIQKMNDARAKWSAFYEEQSRIAKTYPLDEIQTLILKRQLQALQQSGTSGLSADKSKRLNTILNTMSTIYSSGKVLDPNNPQECLVLEPGLDEIMENSKDYSRRLWAWESWRAEVGKQLRPLYEEYVVLENEMARANNYEDYGDYWRGDYEVTGTGDYDYSRNQLMEDVERTFAEIKPLYEHLHAYVRAKLMDAYPSRISPTGCLPAHLLGDMWGRFWTNLYPLTVPFGEKPSIDVTEAMVNQSWDAIRIFEEAEKFFVSIGLPNMTQGFWNNSMLTEPGDGRKVVCHPTAWDLGKGDFRIKMCTKVTMDDFLTAHHEMGHIQYDMAYAIQPYLLRNGANEGFHEAVGEIMSLSAATPHYLKALGLLPPDFYEDSETEINFLLKQALTIVGTLPFTYMLEKWRWMVFKGEIPKEQWMQKWWEMKREIVGVVEPLPHDETYCDPACLFHVAEDYSFIRYYTRTIYQFQFHEALCRTAKHEGPLYKCDISNSTEAGQKLLQMLSLGKSEPWTLALENIVGVKTMDVKPLLSYFEPLLTWLKAQNGNSSVGWNTDWTPYADQSIKVRISLKSALGKEAYEWNDNEMYLFRSSIAYAMRNYFSSAKNETIPFGAEDVWVSDLKPRISFNFFVTSPANMSDIIPRSDVEKAISMSRSRINDAFRLDDNTLEFLGIQPTLGPPDEPPVTVWLIIFGVVMGLVVVGIVVLIFTGIRDRRKKKQASSEENPYGSMDLSKGESNSGFQNGDDIQTSF

SEQ ID NO: 25 - protein, 723 aa, Sus scrofa
QSTTEELAKTFLEKFNLEAEDLAYQSSLASWNYNTNITDENIQKMNDARAKWSAFYEEQSRIAKTYPLDEIQTLILKRQLQALQQSGTSGLSADKSKRLNTILNTMSTIYSSGKVLDPNNPQECLVLEPGLDEIMENSKDYSRRLWAWESWRAEVGKQLRPLYEEYVVLENEMARANNYEDYGDYWRGDYEVTGTGDYDYSRNQLMEDVERTFAEIKPLYEHLHAYVRAKLMDAYPSRISPTGCLPAHLLGDMWGRFWTNLYPLTVPFGEKPSIDVTEAMVNQSWDAIRIFEEAEKFFVSIGLPNMTQGFWNNSMLTEPGDGRKVVCHPTAWDLGKGDFRIKMCTKVTMDDFLTAHHEMGHIQYDMAYAIQPYLLRNGANEGFHEAVGEIMSLSAATPHYLKALGLLPPDFYEDSETEINFLLKQALTIVGTLPFTYMLEKWRWMVFKGEIPKEQWMQKWWEMKREIVGVVEPLPHDETYCDPACLFHVAEDYSFIRYYTRTIYQFQFHEALCRTAKHEGPLYKCDISNSTEAGQKLLQMLSLGKSEPWTLALENIVGVKTMDVKPLLSYFEPLLTWLKAQNGNSSVGWNTDWTPYADQSIKVRISLKSALGKEAYEWNDNEMYLFRSSIAYAMRNYFSSAKNETIPFGAEDVWVSDLKPRISFNFFVTSPANMSDIIPRSDVEKAISMSRSRINDAFRLDDNTLEFLGIQPTLGPPDEPPVT

SEQ ID NO: 26 - protein, 200 aa, SARS-CoV-2
PNITNLCPFGEVFNATRFASVYAWNRKRISNCVADYSVLYNSASFSTFKCYGVSPTKLNDLCFTNVYDSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKS

SEQ ID NO: 27 - protein, 710 aa, SARS-CoV-2
MLLVNQSHQGFNKEHTSKMVSAIVLYVLLAAAAHSAFAQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAR

SEQ ID NO: 28 - protein, 203 aa, SARS-CoV
MNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATYLSLNTAAAL

SEQ ID NO: 29 - protein, 805 aa, Homo sapiens [sequence omitted]

SEQ ID NO: 30 - protein, 723 aa, Homo sapiens
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDRAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 31 - protein, 805 aa, Rhinolophus sinicus
MSSSSWLLLSLVAVTTAQFTTEDLAKIFLDEFNSEAENLSYQSSLASWDYNTNINDENVQKMDEAGAKWSAFYEEQSKLAKNYPLEQIQNVTVKLQLQILQQSGSPVLSEDKSKRLNSILNAMSTIYSTGKVCKPNKPHECLLLEPGLDNIMGTSKDYSERLWAWEGWRAEVGKQLRPLYEEYVVLKNEMARGYHYEDYGDYWRRDYETEESPGPGYSRDQLMKDVERIFTEIKPLYEHLHAYVRAKLMDTYPFHISPTGCLPAHLLGDMWGRFWTNLYPLTVPFGQKPNIDVTDEMLKQGWDADRIFKEAEKFFVSVGLPNMTEGFWNNSMLTEPGDGRKVVCHPTAWDLGKGDFRIKMCTKVTMEDFLTAHHEMGHIQYDMAYASQPYLLRNGANEGFHEAVGEVMSLSVATPKHLKTMGLLSPDFREDNETEINFLLKQALNIVGTLPFTYMLEKWRWMVFKGEIPKEEWMKKWWEMKRKIVGVVEPVPHDETYCDPASLFHVANDYSFIRYYTRTIFEFQFHEALCRIAQHDGPLHKCDISNSTDAGKKLHQMLSVGKSQAWTKTLEDIVDSRNMDVGPLLRYFEPLYTWLQEQNRKSYVGWNTDWSPYSDQSIKVRISLKSALGENAYEWNDNEMYLFRSSVAYAMREYFLKEKHQTILFGAENVWVSNLKPRISFNFHVTSPGNLSDIIPRPEVEGAIRMSRSRINDAFRLDDNSLEFLGIQPTLGPPYQPPVTIWLIVFGVVMAVVVVGIVVLIITGIRDRRKTDQARSEENPYSSVDLSKGENNPGFQNGDDVQTSF

SEQ ID NO: 32 - protein, 723 aa, Rhinolophus sinicus
QFTTEDLAKIFLDEFNSEAENLSYQSSLASWDYNTNINDENVQKMDEAGAKWSAFYEEQSKLAKNYPLEQIQNVTVKLQLQILQQSGSPVLSEDKSKRLNSILNAMSTIYSTGKVCKPNKPHECLLLEPGLDNIMGTSKDYSERLWAWEGWRAEVGKQLRPLYEEYVVLKNEMARGYHYEDYGDYWRRDYETEESPGPGYSRDQLMKDVERIFTEIKPLYEHLHAYVRAKLMDTYPFHISPTGCLPAHLLGDMWGRFWTNLYPLTVPFGQKPNIDVTDEMLKQGWDADRIFKEAEKFFVSVGLPNMTEGFWNNSMLTEPGDGRKVVCHPTAWDLGKGDFRIKMCTKVTMEDFLTAHHEMGHIQYDMAYASQPYLLRNGANEGFHEAVGEVMSLSVATPKHLKTMGLLSPDFREDNETEINFLLKQALNIVGTLPFTYMLEKWRWMVFKGEIPKEEWMKKWWEMKRKIVGVVEPVPHDETYCDPASLFHVANDYSFIRYYTRTIFEFQFHEALCRIAQHDGPLHKCDISNSTDAGKKLHQMLSVGKSQAWTKTLEDIVDSRNMDVGPLLRYFEPLYTWLQEQNRKSYVGWNTDWSPYSDQSIKVRISLKSALGENAYEWNDNEMYLFRSSVAYAMREYFLKEKHQTILFGAENVWVSNLKPRISFNFHVTSPGNLSDIIPRPEVEGAIRMSRSRINDAFRLDDNSLEFLGIQPTLGPPYQPPVT

SEQ ID NO: 33 - protein, 805 aa, Sus scrofa
MSGSFWLLLSLIPVTAAQSTTEELAKTFLEKFNLEAEDLAYQSSLASWTINTNITDENIQKMNDARAKWSAFYEEQSRIAKTYPLDEIQTLILKRQLQALQQSGTSGLSADKSKRLNTILNTMSTIYSSGKVLDPNNPQECLVLEPGLDEIMENSKDYSRRLWAWESWRAEVGKQLRPLYEEYVVLENEMARANNYEDYGDYWRGDYEVTGTGDYDYSRNQLMEDVERTFAEIKPLYEHLHAYVRAKLMDAYPSRISPTGCLPAHLLGDMWGRFWTNLYPLTVPFGEKPSIDVTEAMVNQSWDAIRIFEEAEKFFVSIGLPNMTQGFWNNSMLTEPGDGRKVVCHPTAWDLGKGDFRIKMCTKVTMDDFLTAHHEMGHIQYDMAYAIQPYLLRNGANEGFHEAVGEIMSLSAATPHYLKALGLLPPDFYEDSETEINFLLKQALTIVGTLPFTYMLEKWRWMVFKGEIPKEQWMQKWWEMKREIVGVVEPLPHDETYCDPACLFHVAEDYSFIRYYTRTIYQFQFHEALCRTAKHEGPLYKCDISNSTEAGQKLLQMLSLGKSEPWTLALENIVGVKTMDVKPLLSYFEPLLTWLKAQNGNSSVGWNTDWTPYADQSIKVRISLKSALGEDAYEWNDNEMYLFRSSIAYAMRNYFSSAKNETIPFGAVDVWVSDLKPRISFNFFVTSPANMSDIIPRSDVEKAISMSRSRINDAFRLDDNTLEFLGIQPTLGPPDEPPVTVWLIIFGVVMGLVVVGIVVLIFTGIRDRRKKKQASSEENPYGSMDLSKGESNSGFQNGDDIQTSF

SEQ ID NO: 34 - protein, 723 aa, Sus scrofa
QSTTEELAKTFLEKFNLEAEDLAYQSSLASWTINTNITDENIQKMNDARAKWSAFYEEQSRIAKTYPLDEIQTLILKRQLQALQQSGTSGLSADKSKRLNTILNTMSTIYSSGKVLDPNNPQECLVLEPGLDEIMENSKDYSRRLWAWESWRAEVGKQLRPLYEEYVVLENEMARANNYEDYGDYWRGDYEVTGTGDYDYSRNQLMEDVERTFAEIKPLYEHLHAYVRAKLMDAYPSRISPTGCLPAHLLGDMWGRFWTNLYPLTVPFGEKPSIDVTEAMVNQSWDAIRIFEEAEKFFVSIGLPNMTQGFWNNSMLTEPGDGRKVVCHPTAWDLGKGDFRIKMCTKVTMDDFLTAHHEMGHIQYDMAYAIQPYLLRNGANEGFHEAVGEIMSLSAATPHYLKALGLLPPDFYEDSETEINFLLKQALTIVGTLPFTYMLEKWRWMVFKGEIPKEQWMQKWWEMKREIVGVVEPLPHDETYCDPACLFHVAEDYSFIRYYTRTIYQFQFHEALCRTAKHEGPLYKCDISNSTEAGQKLLQMLSLGKSEPWTLALENIVGVKTMDVKPLLSYFEPLLTWLKAQNGNSSVGWNTDWTPYADQSIKVRISLKSALGEDAYEWNDNEMYLFRSSIAYAMRNYFSSAKNETIPFGAVDVWVSDLKPRISFNFFVTSPANMSDIIPRSDVEKAISMSRSRINDAFRLDDNTLEFLGIQPTLGPPDEPPVT

(19) United States
(12) Patent Application Publication
(10) Pub. No.: US 2022/0026428 A1
(43) Pub. Date: Jan. 27, 2022

(54) DETECTION OF ANTIBODIES TO SARSR-COV

(71) Applicant: National University of Singapore, Singapore (SG)

(72) Inventor: Linfa WANG, Singapore (SG)

(21) Appl. No.: 17/444,223

(22) Filed: Aug. 2, 2021

Related U.S. Application Data
(63) Continuation-in-part of application No. 16/939,405, filed on Jul. 27, 2020, now Pat. No. 11,112,412.

(30) Foreign Application Priority Data
Mar. 25, 2020 (SG) 10202002784P
May 14, 2020 (SG) 10202004468Q

(51) Int. Cl. G01N 33/569

(52) U.S. Cl. CPC G01N 33/56983; G01N 2333/165

(57) ABSTRACT

A kit, composition and method for detection of antibodies to severe acute respiratory syndrome related coronavirus (SARSr-CoV), and for diagnosis of SARSr-CoV infection.

### RELATED APPLICATIONS AND INCORPORATION BY REFERENCE

This application is a continuation-in-part of application Ser. No. 16/939,405 filed Jul. 7, 2020, now allowed, which application claims priority to Singapore Patent Application No. 10202002784P filed Mar. 25, 2020 and Singapore Patent Application No. 10202004468Q filed on May 14, 2020.

The foregoing applications, and all documents cited therein or during their prosecution (“appln cited documents”) and all documents cited or referenced herein (including without limitation all literature documents, patents, published patent applications cited herein) (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference. Any Genbank sequences mentioned in this disclosure are incorporated by reference with the Genbank sequence to be that of the earliest effective filing date of this disclosure.

Citation or identification of any document in this application is not an admission that such document is available as prior art to the present disclosure.

### SEQUENCE STATEMENT

The instant application contains a Sequence Listing, which has been submitted electronically and is hereby incorporated by reference in its entirety. Said ASCII copy, is named 55563_00007SL.txt and is 168 kb in size.

### FIELD OF THE INVENTION

The present disclosure relates to the detection of antibodies to severe acute respiratory syndrome-related coronavirus (SARSr-CoV), and the diagnosis of SARSr-CoV infection.

### BACKGROUND OF THE INVENTION

The coronavirus disease 2019 (COVID-19) outbreak, started in Wuhan, China, has spread rapidly to other cities in China, and affected more than 160 countries with more than 332,930 infections and 14,509 deaths as of 23 Mar. 2020. The etiological agent is severe acute respiratory syndrome coronavirus 2 (SARS-CoV-2), which is genetically 80% identical to severe acute respiratory syndrome coronavirus (SARS-CoV or SARS-CoV-1) which caused the 2002-2003 outbreak of severe acute respiratory syndrome [3].

Current diagnosis is based on clinical history and chest radiographic findings, and confirmation currently relies on nucleic acid-based assays.

There is an urgent need for reliable and easy-to-use assays for the detection of SARS-CoV-2, to expand the tools available to combat this worst pandemic in mankind's modern history.

Unlike molecular tests, serological tests are more difficult to develop and often suffer from specificity issues. The current “gold standard” is virus neutralization test (VNT) which requires the use of live virus in a specialized biocontainment facility, biosafety level 3 (BSL3) lab. The test process is not only expensive and at a biosafety risk, but also slow, typically taking 3-5 days to get results.

Several groups are developing ELISA-based tests to meet the need for BSL2-based tests which can be completed within hours rather than days. The present inventor was the first to use ELISA and VNT assays to facilitate contact tracing in Singapore [30]. Since then, several other groups have developed ELISA tests for SARS-CoV-2 infection [31, 32]. Most groups are using indirect ELISA format (coating ELISA plate with antigen, followed by binding with test sera, and HRP-conjugated secondary antibodies) (FIG. 1). This is the simplest/cheapest serological assay format, hence widely adopted. But it usually suffers from specificity issues. ELISA tests are currently used for “front line” screenings and there is usually a need for confirmation by the more specific and reliable VNT assays [30].

All current ELISA tests are species- (mostly human) and subtype- (IgG or IgM) specific. This can be a major drawback for surveillance studies beyond human sera. There is an urgent need for serological surveillance of different wildlife species to understand the missing link which might be responsible for the transmission of the virus from animal(s) to humans. It is a huge undertaking to develop specific ELISAs for all of the many wildlife species to be surveyed.

### SUMMARY OF THE INVENTION

In a first aspect, the present disclosure provides a kit which may comprise: (i) a polypeptide encoded by a SARSr-CoV or a fragment thereof, (ii) a polypeptide which binds specifically to the polypeptide or fragment of (i), and (iii) means for detecting interaction between the polypeptide or fragment of (i), and the polypeptide of (ii).

Also provided is a kit which may comprise: (i) a spike protein or a fragment thereof encoded by a SARSr-CoV, and (ii) an ACE2 protein or a fragment thereof which binds specifically to the spike protein or fragment of (i).

In some embodiments, the kit may further comprise means for detecting interaction between the spike protein or fragment of (i) and the ACE2 protein or fragment of (ii).

In some embodiments, the spike protein or fragment of (i) or the ACE2 protein or fragment of (ii) may be conjugated to a detection entity. In some embodiments, the spike protein or fragment of (i) or the ACE2 protein or fragment of (ii) may be immobilised on a solid support.

In some embodiments,

(a) the spike protein or fragment of (i) is conjugated to a detectable entity, and wherein the ACE2 or fragment of (ii) is immobilised on a solid support; or (b) the ACE2 or fragment of (ii) is conjugated to a detectable entity, and wherein the spike protein or fragment of (i) is immobilised on a solid support.

In some embodiments, the spike protein or fragment of (i) or the ACE2 protein or fragment of (ii) may be conjugated to a biotin molecule. The biotin molecule may bind an avidin or streptavidin-conjugated detection entity. In some embodiments, the kit of the disclosure may further comprise the avidin or streptavidin-conjugated detection entity. In some embodiments, the spike protein or fragment of (i) or the ACE2 protein or fragment of (ii) may be immobilised on a solid support.

In some embodiments,

(a) the spike protein or fragment of (i) is conjugated to a biotin molecule, and wherein the ACE2 or fragment of (ii) is immobilised on a solid support; or

(b) the ACE2 or fragment of (ii) is conjugated to a biotin molecule, and wherein the spike protein or fragment of (i) is immobilised on a solid support.

In some embodiments, the SARSr-CoV may be SARS-CoV-2 or a variant thereof.

In some embodiments, the spike protein or fragment may comprise or consist of the S1 subunit. In some embodiments, the spike protein or fragment may comprise or consist of an amino acid sequence having at least 70%, 75%, 80%, 85%, 90%, 91%, 92%, 93%, 94%, 95%, 96%, 97%, 98% or 99% sequence identity to SEQ ID NOs: 12 or 27. In some embodiments, the spike protein or fragment may comprise or consist of the receptor binding domain (RBD) of the spike protein. In some embodiments the spike protein or fragment may comprise or consist of an amino acid sequence having at least 70%, 75%, 80%, 85%, 90%, 91%, 92%, 93%, 94%, 95%, 96%, 97%, 98% or 99% sequence identity to SEQ ID NOs: 13 or 26. In some embodiments, the spike protein or fragment may comprise or consist of the receptor binding motif (RBM) of the spike protein. In some embodiments the spike protein or fragment may comprise or consist of an amino acid sequence having at least 70%, 75%, 80%, 85%, 90%, 91%, 92%, 93%, 94%, 95%, 96%, 97%, 98% or 99% sequence identity to SEQ ID NO: 14.

In some embodiments, the ACE2 protein or fragment may comprise or consist of an amino acid sequence having at least 70%, 75%, 80%, 85%, 90%, 91%, 92%, 93%, 94%, 95%, 96%, 97%, 98% or 99% sequence identity to SEQ ID NOs: 15, 16 or 29. In some embodiments, the ACE2 protein or fragment may comprise or consist of the extracellular domain. In some embodiments, the ACE2 protein or fragment may comprise or consist of an amino acid sequence having at least 70%, 75%, 80%, 85%, 90%, 91%, 92%, 93%, 94%, 95%, 96%, 97%, 98% or 99% sequence identity to SEQ ID NOs: 17 or 30.

In some embodiments, the detection entity may be a horseradish peroxidase, an alkaline phosphatase, an acridinium compound, a phycoerythrin, or a fluorescein isothiocyanate.

In some embodiments, the detection entity may be a horseradish peroxidase.

In some embodiments, the detection entity may be a colloidal gold nanoparticle, a colloidal silver nanoparticle, a fluorescent microsphere, or a quantum dot.

The solid support may be a microtiter plate, a bead, or a test strip.

The bead may be a magnetic bead.

The test strip may be a lateral flow assay test strip, or a thin layer chromatography test strip. The test strip, such as the lateral flow assay test strip, may comprise a first absorbent pad, a membrane strip and a second absorbent pad defining a flow path for transporting a sample, with a detection region at the membrane strip. In some embodiments, the solid support is a detection region in a test strip, made of e.g., cellulose acetate or nitrocellulose.

In some embodiments, the detection entity is a horseradish peroxidase, an alkaline phosphatase, an acridinium compound, a phycoerythrin, or a fluorescein isothiocyanate, and the solid support is a microtiter or a bead.

In some embodiments, the detection entity is a colloidal gold nanoparticle, a colloidal silver nanoparticle, a fluorescent microsphere, or a quantum dot, and the solid support is a test strip.

The present disclosure also provides a composition which may comprise:

(a) a solid support, and

(b) (i) a polypeptide encoded by a SARSr-CoV or a fragment thereof, and/or (ii) a polypeptide which binds specifically to the polypeptide or fragment of (i).

The present disclosure also provides a composition which may comprise:

(a) a solid support, and

(b) (i) a spike protein or a fragment thereof encoded by a SARSr-CoV, and/or (ii) an ACE2 protein or a fragment thereof which binds specifically to the spike protein or fragment of (i).

In some embodiments, the composition may further comprise means for detecting interaction between the spike protein or fragment of (i) and the ACE2 protein or fragment of (ii).

In some embodiments, the spike protein or fragment of (i) or the ACE2 protein or fragment of (ii) may be immobilised on the solid support.

In some embodiments, the spike protein or fragment of (i) or the ACE2 protein or fragment of (ii) may be conjugated to a detection entity. In some embodiments, the spike protein or fragment of (i) or the ACE2 protein or fragment of (ii) may be conjugated to a biotin molecule. The biotin molecule may bind to an avidin or streptavidin-conjugated detection entity. In some embodiments, the composition of the disclosure may further comprise the avidin or streptavidin-conjugated detection entity.

In some embodiments,

(a) the spike protein or fragment of (i) is conjugated to a detectable entity, and wherein the ACE2 or fragment of (ii) is immobilised on the solid support; or

(b) the ACE2 or fragment of (ii) is conjugated to a detectable entity, and wherein the spike protein or fragment of (i) is immobilised on the solid support.

In some embodiments,

(a) the spike protein or fragment of (i) is conjugated to a biotin molecule, and wherein the ACE2 or fragment of (ii) is immobilised on a solid support; or

(b) the ACE2 or fragment of (ii) is conjugated to a biotin molecule, and wherein the spike protein or fragment of (i) is immobilised on a solid support. In some embodiments, the SARSr-CoV may be SARS-CoV-2 or a variant thereof.

In some embodiments, the spike protein or fragment may comprise or consist of the S1 subunit. In some embodiments, the spike protein or fragment may comprise or consist of an amino acid sequence having at least 70%, 75%, 80%, 85%, 90%, 91%, 92%, 93%, 94%, 95%, 96%, 97%, 98% or 99% sequence identity to SEQ ID NOs: 12 or 27. In some embodiments, the spike protein or fragment may comprise or consist of the receptor binding domain (RBD) of the spike protein. In some embodiments, the spike protein or fragment may comprise or consist of an amino acid sequence having at least 70%, 75%, 80%, 85%, 90%, 91%, 92%, 93%, 94%, 95%, 96%, 97%, 98% or 99% sequence identity to SEQ ID NOs: 13 or 26. In some embodiments, the spike protein or fragment may comprise or consist of the receptor binding motif (RBM) of the spike protein. In some embodiments the spike protein or fragment may comprise or consist of an amino acid sequence having at least 70%, 75%, 80%, 85%, 90%, 91%, 92%, 93%, 94%, 95%, 96%, 97%, 98% or 99% sequence identity to SEQ ID NO: 14.

In some embodiments, the ACE2 protein or fragment may comprise or consist of an amino acid sequence having at least 70%, 75%, 80%, 85%, 90%, 91%, 92%, 93%, 94%, 95%, 96%, 97%, 98% or 99% sequence identity to SEQ ID NOs: 15, 16 or 29. In some embodiments, the ACE2 protein or fragment may comprise or consist of the extracellular domain. In some embodiments, the ACE2 protein or fragment may comprise or consist of an amino acid sequence having at least 70%, 75%, 80%, 85%, 90%, 91%, 92%, 93%, 94%, 95%, 96%, 97%, 98% or 99% sequence identity to SEQ ID NOs: 17 or 30.

The detection entity may be a horseradish peroxidase, an alkaline phosphatase, an acridinium compound, a phycoerythrin, or a fluorescein isothiocyanate.

In some embodiments the detection entity may be a horseradish peroxidase.

In some embodiments, the detection entity may be a colloidal gold nanoparticle, a colloidal silver nanoparticle, a fluorescent microsphere, or a quantum dot. The solid support may be a microtiter plate, a bead, or a test strip.

The bead may be a magnetic bead.

The test strip may be a lateral flow assay test strip, or a thin layer chromatography test strip. The test strip, such as the lateral flow assay test strip, may comprise a first absorbent pad, a membrane strip and a second absorbent pad defining a flow path for transporting a sample, with a detection region at the membrane strip. In some embodiments, the solid support is a detection region in a test strip, made of e.g., cellulose acetate or nitrocellulose.

In some embodiments, the detection entity is a horseradish peroxidase, an alkaline phosphatase, an acridinium compound, a phycoerythrin, or a fluorescein isothiocyanate, and the solid support is a microtiter or a bead.

In some embodiments, the detection entity is a colloidal gold nanoparticle, a colloidal silver nanoparticle, a fluorescent microsphere, or a quantum dot, and the solid support is a test strip.

The present disclosure also provides a polypeptide which may comprise the amino acid sequence of a polypeptide encoded by a SARSr-CoV or a fragment thereof, wherein the polypeptide may be conjugated to a detection entity or a biotin molecule. The biotin molecule may bind an avidin or streptavidin-conjugated detection entity.

The present disclosure also provides a polypeptide which may comprise the amino acid sequence of a spike protein or a fragment thereof encoded by a SARSr-CoV, wherein the polypeptide may be conjugated to a detection entity or a biotin molecule. The biotin molecule may bind an avidin or streptavidin-conjugated detection entity.

In some embodiments, the SARSr-CoV may be SARS-CoV-2 or a variant thereof.

In some embodiments, the spike protein or fragment may comprise or consist of the S1 subunit. In some embodiments, the spike protein or fragment may comprise or consist of an amino acid sequence having at least 70%, 75%, 80%, 85%, 90%, 91%, 92%, 93%, 94%, 95%, 96%, 97%, 98% or 99% sequence identity to SEQ ID NOs: 12 or 27. In some embodiments, the spike protein or fragment may comprise or consist of the receptor binding domain (RBD) of the spike protein. In some embodiments, the spike protein or fragment may comprise or consist of an amino acid sequence having at least 70%, 75%, 80%, 85%, 90%, 91%, 92%, 93%, 94%, 95%, 96%, 97%, 98% or 99% sequence identity to SEQ ID NOs: 13 or 26. In some embodiments, the spike protein or fragment may comprise or consist of the receptor binding motif (RBM) of the spike protein. In some embodiments the spike protein or fragment may comprise or consist of an amino acid sequence having at least 70%, 75%, 80%, 85%, 90%, 91%, 92%, 93%, 94%, 95%, 96%, 97%, 98% or 99% sequence identity to SEQ ID NO: 14.

The present disclosure also provides a polypeptide which may bind specifically to a polypeptide encoded by a SARSr-CoV or a fragment thereof, wherein the polypeptide may be conjugated to a detection entity or a biotin molecule. The biotin molecule may bind an avidin or streptavidin-conjugated detection entity.

The present disclosure also provides a polypeptide which may comprise the amino acid sequence of an ACE2 protein or a fragment thereof, wherein the polypeptide may be conjugated to a detection entity or a biotin molecule. The biotin molecule may bind an avidin or streptavidin-conjugated detection entity.

In some embodiments, the ACE2 protein or fragment may comprise or consist of an amino acid sequence having at least 70%, 75%, 80%, 85%, 90%, 91%, 92%, 93%, 94%, 95%, 96%, 97%, 98% or 99% sequence identity to SEQ ID NOs: 15, 16 or 29. In some embodiments, the ACE2 protein or fragment may comprise or consist of the extracellular domain. In some embodiments, the ACE2 protein or fragment may comprise or consist of an amino acid sequence having at least 70%, 75%, 80%, 85%, 90%, 91%, 92%, 93%, 94%, 95%, 96%, 97%, 98% or 99% sequence identity to SEQ ID NOs: 17 or 30.

In some embodiments, the detection entity may be a horseradish peroxidase, an alkaline phosphatase, an acridinium compound, a phycoerythrin, or a fluorescein isothiocyanate.

In some embodiments, the detection entity may be a horseradish peroxidase.

In some embodiments, the detection entity may be a colloidal gold nanoparticle, a colloidal silver nanoparticle, a fluorescent microsphere, or a quantum dot.

The quantity of the spike protein or fragment of (i), the ACE2 protein or fragment of (ii), or the polypeptide, of the disclosure that is immobilized on the solid support may depend on a) the quantity of the spike protein or fragment of (i), the ACE2 protein or fragment of (ii), or the polypeptide, that is incubated with/impregnated in the solid support, and/or b) the capacity of the solid support to accommodate the spike protein or fragment of (i), the ACE2 protein or fragment of (ii) or the polypeptide. In some embodiments, the quantity of the spike protein or fragment of (i), the ACE2 protein or fragment of (ii), or the polypeptide to be immobilized on a microtiter plate (or a plate well) is determined by e.g., a) the quantity of the spike protein or fragment of (i), the ACE2 protein or fragment of (ii), or the polypeptide, that is incubated with the microtiter plate, and b) the area of a microtiter plate (or a plate well) that is for incubating and attaching the spike protein or fragment of (i), the ACE2 protein or fragment of (ii), or the polypeptide. In some embodiments, the quantity of the spike protein or fragment of (i), the ACE2 protein or fragment of (ii), or the polypeptide to be immobilized on beads, e.g., magnetic beads, is determined by e.g., a) the quantity of the spike protein or fragment of (i), the ACE2 protein or fragment of (ii), or the polypeptide, that is incubated with the beads, b) the quantity of beads incubated with the spike protein or fragment of (i), the ACE2 protein or fragment of (ii) or the polypeptide, and c) the space, e.g., the surface area, of a bead for conjugating the spike protein or fragment of (i), the ACE2 protein or fragment of (ii), or the polypeptide. In some embodiments, the quantity of the spike protein or fragment of (i), the ACE2 protein or fragment of (ii), or the polypeptide, of the disclosure that is immobilized on the bead may be regulated by adjusting a) the quantity/concentration of the spike protein or fragment of (i), the ACE2 protein or fragment of (ii), or the polypeptide, of the disclosure that is incubated with the bead, b) the quantity/concentration of the beads incubated with the spike protein or fragment of (i), the ACE2 protein or fragment of (ii), or the polypeptide, of the disclosure, and/or c) the surface area of a bead for conjugating the spike protein or fragment of (i), the ACE2 protein or fragment of (ii), or the polypeptide. In some embodiments, the quantity of the spike protein or fragment of (i), the ACE2 protein or fragment of (ii), or the polypeptide, of the disclosure that is immobilized on the test strip is determined by e.g., the quantity of the spike protein or fragment of (i), the ACE2 protein or fragment of (ii), or the polypeptide, that is impregnated in or coated on the test strip.

The present disclosure also provides a nucleic acid encoding a polypeptide according to the present disclosure.

The present disclosure also provides a vector which may comprise the nucleic acid according to the present disclosure.

The present disclosure also provides a cell which may comprise the nucleic acid or vector according to the present disclosure.

The present disclosure also provides the use of the kit, composition or polypeptide according to the present disclosure, in a method for detecting the presence of antibodies to a SARSr-CoV in a sample.

In some embodiments, the SARSr-CoV may be SARS-CoV-2 or a variant thereof.

The present disclosure also provides the use of the kit, composition or polypeptide according to the present disclosure, in a method for determining whether a subject is or has been infected with a SARSr-CoV.

In some embodiments, the SARSr-CoV may be SARS-CoV-2 or a variant thereof.

In some embodiments, the subject may be a mammal. In some embodiments, the subject may be a human.

The present disclosure also provides a method of analysing a sample for the presence of antibodies to a SARSr-CoV, which may comprise:
contacting the sample with: (i) a polypeptide encoded by a SARSr-CoV or a fragment thereof, and (ii) a polypeptide which binds specifically to the polypeptide or fragment of (i), and
determining the level of interaction between the polypeptide or fragment of (i) and the polypeptide of (ii).

In some embodiments, the SARSr-CoV may be SARS-CoV-2 or a variant thereof.

In some embodiments, the sample may be a blood sample, a lymph sample, a saliva sample, a synovial fluid sample. In some embodiments, the sample may be serum.

The present disclosure also provides a method of determining whether a subject is or has been infected with a SARSr-CoV, which may comprise:
contacting a sample from the subject with: (i) a polypeptide encoded by a SARSr-CoV or a fragment thereof, and (ii) a polypeptide which binds specifically to the polypeptide or fragment of (i), and
determining the level of interaction between the polypeptide or fragment of (i) and the polypeptide of (ii).

In some embodiments, the SARSr-CoV may be SARS-CoV-2 or a variant thereof.

In some embodiments, the subject may be a mammal. In some embodiments, the subject may be a human.

In some embodiments, the sample may be a blood sample, a lymph sample, a saliva sample, a synovial fluid sample. In some embodiments, the sample may be serum.

The present disclosure also provides a method of analysing a sample for the presence of antibodies to a SARSr-CoV, which may comprise:
contacting the sample with: (i) a spike protein or a fragment thereof encoded by a SARSr-CoV, and (ii) an ACE2 protein or a fragment thereof which binds specifically to the spike protein or fragment of (i), and
determining the level of interaction between the spike protein or fragment of (i) and the ACE2 or fragment of (ii).

The present disclosure also provides a method of determining whether a subject is or has been infected with a SARSr-CoV, which may comprise:
contacting a sample obtained from the subject with: (i) a spike protein or a fragment thereof encoded by a SARSr-CoV, and (ii) an ACE2 protein or a fragment thereof which binds specifically to the spike protein or fragment of (i), and
determining the level of interaction between the spike protein or fragment of (i) and the ACE2 or fragment of (ii).

In some embodiments, the spike protein or fragment of (i) or the ACE2 protein or fragment of (ii) may be conjugated to a detection entity. In some embodiments, the spike protein or fragment of (i) or the ACE2 protein or fragment of (ii) may be conjugated to a biotin molecule, and the biotin molecule may bind an avidin or streptavidin-conjugated detection entity.

In some embodiments, the spike protein or fragment of (i) or the ACE2 protein or fragment of (ii) may be immobilised on a solid support. The solid support may be a microtiter plate, a bead, or a test strip. The bead may be a magnetic bead. The test strip may be a lateral flow assay test strip, or a thin layer chromatography test strip.

In some embodiments, the spike protein or fragment of (i) may be conjugated to a detectable entity, and wherein the ACE2 or fragment of (ii) may be immobilised on a solid support. In some embodiments, the spike protein or fragment of (i) may be conjugated to a biotin molecule, the biotin molecule may bind an avidin or streptavidin-conjugated detection entity, and wherein the ACE2 or fragment of (ii) may be immobilised on a solid support.

In some embodiments, the SARSr-CoV may be SARS-CoV-2 or a variant thereof.

In some embodiments, the subject may be a mammal. In some embodiments, the subject may be a human.

In some embodiments, the sample may be a blood sample, a lymph sample, a saliva sample, a synovial fluid sample. In some embodiments, the sample may be serum.

In some embodiments, the spike protein or fragment may comprise or consist of the S1 subunit. In some embodiments, the spike protein or fragment comprises or consists of an amino acid sequence having at least 70%, 75%, 80%, 85%, 90%, 91%, 92%, 93%, 94%, 95%, 96%, 97%, 98% or 99% sequence identity to SEQ ID NOs: 12 or 27. In some embodiments, the spike protein or fragment may comprise or consist of the receptor binding domain (RBD) of the spike protein. In some embodiments the spike protein or fragment may comprise or consist of an amino acid sequence having at least 70%, 75%, 80%, 85%, 90%, 95% or 99% sequence identity to SEQ ID NOs: 13 or 26. In some embodiments, the spike protein or fragment may comprise or consist of the receptor binding motif (RBM) of the spike protein. In some embodiments the spike protein or fragment may comprise or consist of an amino acid sequence having at least 70%, 75%, 80%, 85%, 90%, 91%, 92%, 93%, 94%, 95%, 96%, 97%, 98% or 99% sequence identity to SEQ ID NO: 14.

In some embodiments, the ACE2 protein or fragment may comprise or consist of an amino acid sequence having at least 70%, 75%, 80%, 85%, 90%, 91%, 92%, 93%, 94%, 95%, 96%, 97%, 98% or 99% sequence identity to SEQ ID NOs: 15, 16 or 29. In some embodiments, the ACE2 protein or fragment may comprise or consist of the extracellular domain. In some embodiments, the ACE2 protein or fragment may comprise or consist of an amino acid sequence having at least 70%, 75%, 80%, 85%, 90%, 91%, 92%, 93%, 94%, 95%, 96%, 97%, 98% or 99% sequence identity to SEQ ID NOs: 17 or 30.

In some embodiments, the detection entity may be a horseradish peroxidase, an alkaline phosphatase, an acridinium compound, a phycoerythrin, or a fluorescein isothiocyanate.

In some embodiments, the detection entity may be a horseradish peroxidase.

In some embodiments, the detection entity may be a colloidal gold nanoparticle, a colloidal silver nanoparticle, a fluorescent microsphere, or a quantum dot.

In some embodiments, the detection entity is a horseradish peroxidase, an alkaline phosphatase, an acridinium compound, a phycoerythrin, or a fluorescein isothiocyanate, and the solid support is a microtiter or a bead.

In some embodiments, the detection entity is a colloidal gold nanoparticle, a colloidal silver nanoparticle, a fluorescent microsphere, or a quantum dot, and the solid support is a test strip.

In some embodiments, the method of the disclosure comprises:
contacting the sample with the spike protein or fragment of (i),
immobilizing the spike protein or fragment of (i) to the solid support,
contacting the spike protein or fragment of (i) immobilized on the solid support with the ACE2 or fragment of (ii), and
determining the level of interaction between the spike protein or fragment of (i) and the ACE2 or fragment of (ii),

wherein the ACE2 or fragment of (ii) is conjugated to the detection entity.

In some embodiments, the ACE2 or fragment of (ii) is conjugated to a detection entity selected from the group consisting of a hoseradish peroxidase, an alkaline phosphatase, an acridinium compound, a phycoerythrin, and a fluorescein isothiocyanate.

In some embodiments, the spike protein or fragment of (i) is conjugated to a biotin, and the solid support is conjugated with an avidin or a streptavidin. The solid support may be a microtiter plate, or a bead. In some embodiments, the solid support is a microtiter plate, and the method may comprise the steps of coating the microtiter plate with the avidin or streptavidin, and optionally incubating the plate with a blocking buffer to block non-specific binding sites, prior to the step of immobilizing the spike protein or fragment of (i) to the microtiter plate. In some embodiments, the solid support is a bead, and the method may comprise steps of conjugating an avidin or a streptavidin to the bead via e.g., chemical reactions between chemical groups on avidin or streptavidin and chemical groups on the bead, and optionally incubating the bead with a blocking buffer to block non-specific binding sites, prior to the step of immobilizing the spike protein or fragment of (i) to the bead.

In some embodiments, the spike protein or fragment of (i) is conjugated to an avidin or a streptavidin, and the solid support is conjugated with a biotin. The solid support may be a bead, such as a magnetic bead. In some embodiments, the method may comprise the step of conjugating the biotin to the bead via e.g., chemical reactions between chemical groups on biotin and chemical groups on the bead.

The method may comprise the step of removing components that do not bind the spike protein or fragment of (i), prior to the step of contacting the spike protein or fragment of (i) immobilized on the solid support with the ACE2 or fragment of (ii).

In some embodiments, the method of the disclosure comprises:
immobilizing the spike protein or fragment of (i) to the solid support,
contacting the sample with the spike protein or fragment of (i) immobilized on the solid support,
contacting the spike protein or fragment of (i) immobilized on the solid support with the ACE2 or fragment of (ii), and
determining the level of interaction between the spike protein or fragment of (i) and the ACE2 or fragment of (ii),

wherein the ACE2 or fragment of (ii) is conjugated to a) the detection entity or b) the biotin molecule which binds an avidin or streptavidin-conjugated detection entity.

In some embodiments, the solid support is a microtiter plate. The method may comprise the step of coating the microtiter plate with the spike protein or fragment of (i) to immobilize the spike protein or fragment of (i) to the solid support. The method may further comprise the step of incubating the plate with a blocking buffer to block non-specific binding sites, prior to the step of contacting the sample with the spike protein or fragment of (i) immobilized on the solid support. The method may comprise the step of removing components that do not bind the spike protein or fragment of (i), after the step of contacting the sample with the spike protein or fragment of (i) immobilized on the solid support.

In some embodiments, the solid support is a bead, e.g., a magnetic bead. The method may comprise the step of conjugating the spike protein or fragment of (i) to the bead via, e.g., chemical reactions between chemical groups on the spike protein or fragment of (i) and chemical groups on the bead, to immobilize the spike protein or fragment of (i) to the bead. In some embodiments, the method may comprise the step of binding a biotinylated spike protein or fragment of (i) to an avidin or streptavidin-conjugated bead, or alternatively binding an avidin or streptavidin-conjugated spike protein of fragment of (i) to a biotin-conjugated bead, to immobilize the spike protein or fragment of (i) to the bead. The method may comprise the step of removing components that do not bind the spike protein or fragment of (i), after the step of contacting the sample with the spike protein or fragment of (i) immobilized on the solid support.

In some embodiments, the quantity of the spike protein or fragment of (i) immobilized on the solid support may be determined by a) the quantity or concentration of the beads incubated with the spike protein or fragment of (i), b) the quantity or concentration of the spike protein or fragment of (i) incubated with the beads, and/or c) the capacity of a bead to accommodate the spike protein or fragment of (i), such as the surface area of a bead for conjugating the spike protein of fragment of (i). The quantity of the spike protein or fragment of (i) immobilized on the solid support may be regulated by adjusting a) the quantity of the spike protein or fragment of (i) incubated with the bead, b) the quantity of the bead incubated with the spike protein or fragment of (i), and/or c) the space, e.g., the surface area, of a bead for conjugating the spike protein or fragment of (i).

The molar ratio of the spike protein or fragment of (i) to the antibodies to a SARSr-CoV in the sample may be optimized by e.g., adjusting the spike protein or fragment of (i) immobilized on the bead/microtiter plate, such that the method of the disclosure may detect antibodies with higher sensitivity and/or higher specificity. In some embodiments, the method of the disclosure comprises:
immobilizing the spike protein or fragment of (i) to the test strip,
contacting the sample with the ACE2 or fragment of (ii),
transporting the resultant mixture along the test strip, and
determining the level of interaction between the spike protein or fragment of (i) and the ACE2 or fragment of (ii),

wherein the ACE2 or fragment of (ii) is conjugated to a detection entity selected from the group consisting of a colloidal gold nanoparticle, a colloidal silver nanoparticle, a fluorescent microsphere, or a quantum dot.

In some embodiments, the method of the disclosure comprises:
immobilizing the ACE2 or fragment of (ii) to the test strip,
contacting the sample with the spike protein or fragment of (i),
transporting the resultant mixture along the test strip, and
determining the level of interaction between the spike protein or fragment of (i) and the ACE2 or fragment of (ii),

wherein the spike protein or fragment of (i) is conjugated to a detection entity selected from the group consisting of a colloidal gold nanoparticle, a colloidal silver nanoparticle, a fluorescent microsphere, or a quantum dot.

Other features and advantages of the instant disclosure will be apparent from the following detailed description and examples which should not be construed as limiting. The contents of all references, GenBank entries, patents and published patent applications cited throughout this application are expressly incorporated herein by reference.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1. Schematic representation of the principle of indirect ELISA. 1) Antigen coating; 2) Binding of antigen-specific antibody; 3) Binding of HRP-conjugated secondary antibody; 4) Colour development with HRP substrate (TMB).

FIGS. 2A and 2B. Schematic representation of the principle of virus neutralization. (2A) Most virus-neutralizing antibodies act by blocking virus attachment although some can also act by blocking uncoating. (2B) Surrogate virus neutralization mimics the action of most neutralizing antibodies by blocking receptor-viral protein interaction in an ELISA plate well.

FIGS. 3A and 3B. Graphs showing the results of analysis of direct binding to hACE2 coated onto an ELISA plate for (3A) recombinant HRP-conjugated SARS-CoV-2 spike protein RBD, (3B) recombinant HRP-conjugated SARS-CoV-2 spike protein S1 subunit, and recombinant HRP-conjugated SARS-CoV-2 nucleoprotein.

FIGS. 4A and 4B. Graphs showing the results of analysis of serum samples by surrogate virus neutralisation test (sVNT) using immobilised human ACE2 protein and (4A) HRP-conjugated SARS-CoV-2 spike protein RBD or (4B) HRP-conjugated SARS-CoV-2 spike protein S1 subunit.

FIG. 5. Graph showing the results of analysis of 74 serum samples from PCR-confirmed COVID-19 patients and 11 negative (healthy) human serum samples using the sVNT using immobilised human ACE2 protein and HRP-conjugated SARS-CoV-2 spike protein RBD.

FIGS. 6A to 6F. Schematic representations and graphs showing the principle and initial validation of the SARS-CoV-2 surrogate virus neutralization test (sVNT). (6A) Mechanism of conventional virus neutralization test (VNT). Anti-SARS-CoV-2 neutralizing antibodies block SARS-CoV-2 Spike protein from binding to hACE2 receptor proteins on the host cell surface. (6B) In the sVNT assay, anti-SARS-CoV-2 neutralizing antibodies block HRP-conjugated RBD protein from binding to the hACE2 protein pre-coated on an ELISA plate. (6C) Binding of HRP-conjugated SARS-CoV-2 N, S1 and RBD proteins to hACE2. (6D) Inhibition of SARS-CoV-2 RBD-hACE2 interaction by COVID-19 patient sera. (6E) Binding of HRP-conjugated SARS-CoV RBD to hACE2. (6F) Inhibition of SARS-CoV RBD-hACE2 interaction by SARS patient sera.

FIGS. 7A to 7D. Bar charts showing isotype-independent neutralization by human sera with different levels of IgM and IgG antibodies. (7A) High IgM/Low IgG (n=5); (7B) Low IgM/High IgG (n=3); (7C) Low IgM/Low IgG (n=9); (7D) High IgM/High IgG (n=5). The IgM and IgG levels were determined by isotype-specific capture ELISA.

FIGS. 8A to 8G. Graphs showing species-independent and virus-specific neutralization. (8A) Rabbit anti-SARS-CoV-2 RBD sera from immunization (n=3). (8B) Ferret anti-SARS-CoV sera from infection (n=2). (8C) Rabbit anti-SARS-CoV sera from immunization (n=2). (8D) SARS-CoV-2 sVNT using different coronavirus sera: human COVID-19 sera (n=10), human SARS sera sampled in 2003 (n=7, <1 year), human SARS-CoV sera sampled in 2020 (n=10, >17 years), human OC43 sera (n=8), human 229E/NL63 sera (n=10), MERS-CoV sera from experimentally infected alpaca (n=4). (8E) Comparative analysis of homologous and heterologous NAb levels for the 2003 SARS serum panel. (8F) Comparative analysis of homologous and heterologous NAb levels for the 2020 SARS serum panel. (8G) Comparative analysis of homologous N-specific antibodies in the three serum cohorts. SARS-CoV-2 N protein indirect ELISA for COVID-19 sera and SARS-CoV N protein indirect ELISA for the two SARS serum panels.

FIGS. 9A to 9C. Correlation between sVNT and VNT and sVNT testing with two COVID-19 patient cohorts from two different nations. (9A) Correlation analysis for 13 COVID-19 sera with different levels of SARS-CoV-2 antibodies by VNT and sVNT at 70% inhibition. Testing of healthy control and COVID-19 serum cohorts in Singapore (9B) (COVID-19 n=77, control n=75) and Nanjing, China (9C) (COVID-19 n=50, control n=50).

FIG. 10. Table showing correlation between VNT and sVNT for different COVID-19 sera. Neutralization titers were obtained from two biological replicates each with two technical replicates. sVNT titers at 90%, 70%, 50% and 30% inhibition, respectively, are shown.

FIG. 11. Graph showing titration of COVID-19 sera with 417 different levels of SARS-CoV-2 antibodies. Serum samples were two-fold diluted starting at 1:10. Percentage inhibition was plotted at each dilution point for two negative controls and 13 COVID-19 sera from PCR positive patients.

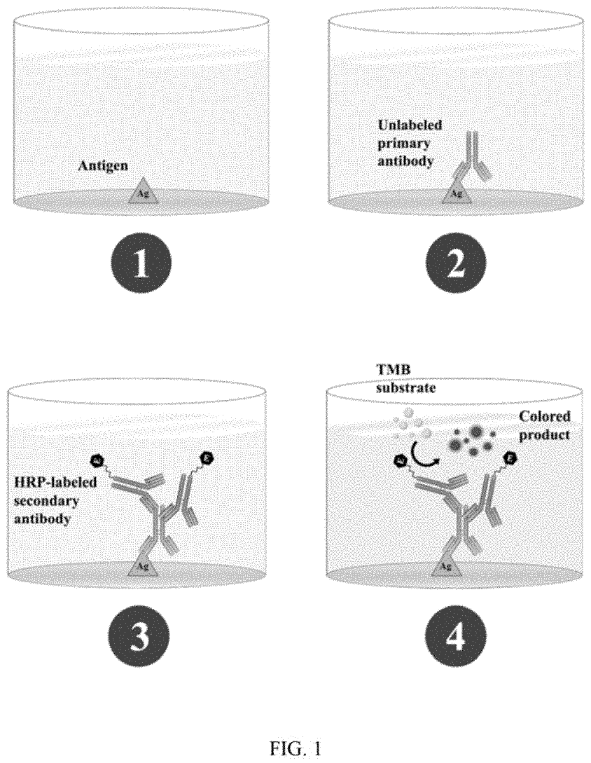

### DETAILED DESCRIPTION OF THE INVENTION

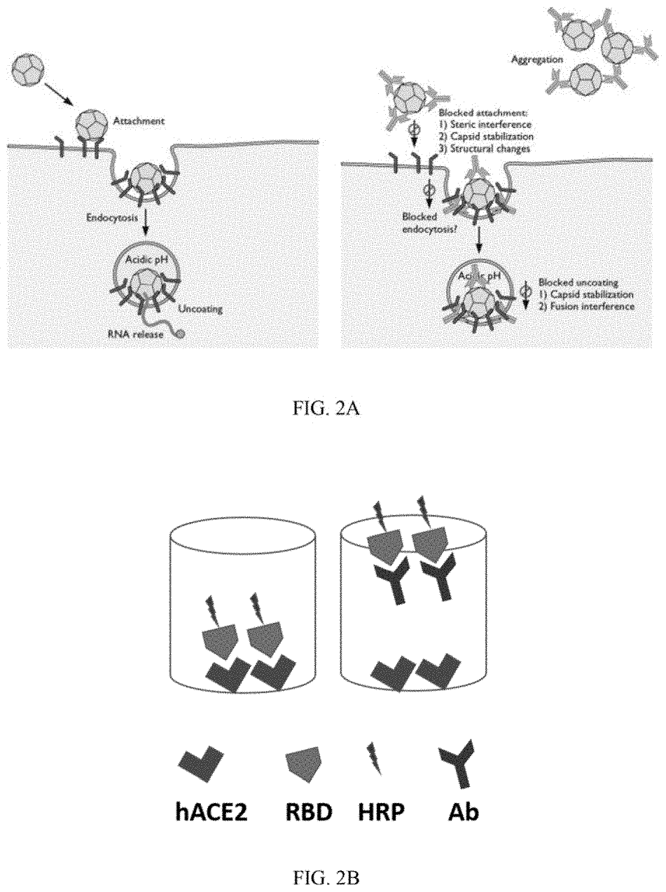

The present invention is based on the principle of using surrogate molecules of live SARSr-CoV in assays for the detection of the presence of neutralising antibody to SARSr-CoV. The invention strips the traditional virus neutralisation test back to its molecular elements, based on antibodies blocking the molecular interaction of the cell entry receptor (ACE2) and the key viral spike protein 51 domain (RBD) (FIG. 2B).

Advantageously, analysis of samples using the articles and in accordance with methods of the present disclosure is more reliable than ELISA-based analysis, and is more versatile, not being limited to the detection of certain antibody isotypes or the detection of antibodies in samples derived from a limited number of species. Rather, the articles and methods of the present disclosure can be employed to detect neutralising antibodies to SARSr-CoV across all antibody isotypes and in samples derived from any species. As the disclosed articles and methods provide for more sensitive and specific analysis, there is no need to confirm results by virus neutralisation tests, as is typically required for ELISA-based analysis.

Moreover, the analysis of samples using the articles and in accordance with methods of the present disclosure is associated with the advantage over virus neutralisation tests that it does not employ live virus or infectious material, and is therefore safe and does not require the use of BSL3 facilities. Furthermore, the use of the articles and methods of the present disclosure is less expensive and time-consuming than virus neutralisation tests, with results obtainable in a matter of hours.

The present disclosure concerns severe acute respiratory syndrome-related coronavirus (SARSr-CoV). The virology of SARSr-CoV and epidemiology of disease associated with SARSr-CoV infection are reviewed, for example, in Cheng et al., Clin Microbiol Rev (2007) 20(4): 660-694 and de Wit et al., Nat Rev Microbiol (2016) 14: 523-534, both of which are hereby incorporated by reference in their entirety.

SARSr-CoV is a species of coronavirus of the genus Betacoronavirus and subgenus Sarbecoronavirus that infects humans, bats and certain other mammals. It is an enveloped positive-sense single-stranded RNA virus.

Two strains of SARSr-CoV have caused serious outbreaks of severe respiratory diseases in humans: SARS-CoV, which caused an outbreak of severe acute respiratory syndrome (SARS) between 2002 and 2003, and SARS-CoV-2, which has caused the coronavirus disease 2019 (COVID-19) pandemic. There are hundreds of strains of SARSr-CoV known only to infect non-human species; bats are a major reservoir of many strains of SARS-related coronaviruses.

In some embodiments in accordance with the various aspects of the present disclosure, the SARSr-CoV is a strain of SARSr-CoV that causes coronavirus disease in humans.

In some embodiments, the SARSr-CoV is SARS-CoV or a variant thereof, or SARS-CoV-2 or a variant thereof. In some embodiments, the SARSr-CoV is SARS-CoV-2 or a variant thereof.

In some embodiments herein, “SARS-CoV” refers to the SARSr-CoV strain having the nucleotide sequence with a GenBank Accession No.: AY278488.2 (“SARS coronavirus BJ01, complete genome”) see, e.g., Wu et al., Genomics Proteomics Bioinformatics 1 (2), 131-144 (2003). A variant of SARS-CoV may comprise a nucleotide sequence having at least 80% sequence identity, e.g. one of at least 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 99.9% sequence identity to the nucleotide sequence with a GenBank Accession No.: AY278488.2.

As used herein, “sequence identity” refers to the percent of nucleotides/amino acid residues in a subject sequence that are identical to nucleotides/amino acid residues in a reference sequence, after aligning the sequences and, if necessary, introducing gaps, to achieve the maximum percent sequence identity between the sequences. Pairwise and multiple sequence alignment for the purposes of determining percent sequence identity between two or more amino acid or nucleic acid sequences can be achieved in various ways known to a person of skill in the art, for instance, using the publicly available computer software such as ClustalOmega (Soding, J. 2005, Bioinformatics 21, 951-960), T-coffee (Notredame et al. 2000, J. Mol. Biol. (2000) 302, 205-217), Kalign (Lassmann and Sonnhammer 2005, BMC Bioinformatics, 6(298)) and MAFFT (Katoh and Standley 2013, Molecular Biology and Evolution, 30(4) 772-780 software. When using such softwares, the default parameters, e.g. for gap penalty and extension penalty, are preferably used.

In some embodiments herein, “SARS-CoV-2” refers to the SARSr-CoV strain having the nucleotide sequence with a GenBank Accession No.: MN996527.1 (“Severe acute respiratory syndrome coronavirus 2 isolate WIV02, complete genome”), reported in Zhou et al., Nature (2020) 579: 270-273. A variant of SARS-CoV-2 may comprise a nucleotide sequence having at least 80% sequence identity, e.g. one of at least 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 99.9% sequence identity to the nucleotide sequence with a GenBank Accession No.: MN996527.1.

In preferred embodiments, the SARSr-CoV is SARS-CoV-2 or a variant thereof.

The present disclosure also concerns polypeptides encoded by a SARSr-CoV.

Herein, a polypeptide refers to any molecule which may comprise three or more amino acid residues joined by peptide bonds. A polypeptide according to the present disclosure encompasses peptides (e.g. tripeptides, oligopeptides, etc.), and also polypeptides which may comprise chemical modification, such e.g. glycosylation (glycopolypeptides), phosphorylation, hydroxylation, sulfonation, palmitoylation, and disulfide formation. Polypeptides may also be referred to as proteins.

Constituent proteins and glycoproteins encoded by the SARSr-CoV genome are described e.g. in Masters, Adv Virus Res. (2006) 66:193-292 and Song et al., Viruses (2019) 11(1): 59, both of which are hereby incorporated by reference in their entirety. The SARSr-CoV genome encodes four major structural proteins: the spike (S) protein, the envelope (E) protein, the membrane (M) protein, and the nucleocapsid (N) protein.

The spike protein of SARS-CoV may have the amino acid sequence shown in SEQ ID NO: 1. The envelope protein of SARS-CoV may have the amino acid sequence shown in SEQ ID NO: 2. The membrane protein of SARS-CoV may have the amino acid sequence shown in SEQ ID NO: 3. The nucleocapsid protein of SARS-CoV may have the amino acid sequence shown in SEQ ID NO: 4.

The spike protein of SARS-CoV-2 may have the amino acid sequence shown in SEQ ID NO: 5. The envelope protein of SARS-CoV-2 may have the amino acid sequence shown in SEQ ID NO: 6. The membrane protein of SARS-CoV-2 may have the amino acid sequence shown in SEQ ID NO: 7. The nucleocapsid protein of SARS-CoV-2 may have the amino acid sequence shown in SEQ ID NO: 8.

In some embodiments, a polypeptide encoded by a SARS-CoV may comprise or consist of the amino acid sequence of SEQ ID NO: 1, or an amino acid sequence having at least 70% sequence identity, e.g. one of at least 80%, 85%, 90%, 91%, 92%, 93%, 94%, 95%, 96%, 97%, 98% or 99% amino acid sequence identity to the amino acid sequence of SEQ ID NO: 1. In some embodiments, a polypeptide encoded by a SARS-CoV may comprise or consist of the amino acid sequence of SEQ ID NO: 2, or an amino acid sequence having at least 70% sequence identity, e.g. one of at least 80%, 85%, 90%, 91%, 92%, 93%, 94%, 95%, 96%, 97%, 98% or 99% amino acid sequence identity to the amino acid sequence of SEQ ID NO: 2. In some embodiments, a polypeptide encoded by a SARS-CoV may comprise or consist of the amino acid sequence of SEQ ID NO: 3, or an amino acid sequence having at least 70% sequence identity, e.g. one of at least 80%, 85%, 90%, 91%, 92%, 93%, 94%, 95%, 96%, 97%, 98% or 99% amino acid sequence identity to the amino acid sequence of SEQ ID NO: 3. In some embodiments, a polypeptide encoded by a SARS-CoV may comprise or consist of the amino acid sequence of SEQ ID NO: 4, or an amino acid sequence having at least 70% sequence identity, e.g. one of at least 80%, 85%, 90%, 91%, 92%, 93%, 94%, 95%, 96%, 97%, 98% or 99% amino acid sequence identity to the amino acid sequence of SEQ ID NO: 4.

In some embodiments, a polypeptide encoded by a SARS-CoV-2 may comprise or consist of the amino acid sequence of SEQ ID NO: 5, or an amino acid sequence having at least 70% sequence identity, e.g. one of at least 80%, 85%, 90%, 91%, 92%, 93%, 94%, 95%, 96%, 97%, 98% or 99% amino acid sequence identity to the amino acid sequence of SEQ ID NO: 5. In some embodiments, a polypeptide encoded by a SARS-CoV-2 may comprise or consist of the amino acid sequence of SEQ ID NO: 6, or an amino acid sequence having at least 70% sequence identity, e.g. one of at least 80%, 85%, 90%, 91%, 92%, 93%, 94%, 95%, 96%, 97%, 98% or 99% amino acid sequence identity to the amino acid sequence of SEQ ID NO: 6. In some embodiments, a polypeptide encoded by a SARS-CoV-2 may comprise or consist of the amino acid sequence of SEQ ID NO: 7, or an amino acid sequence having at least 70% sequence identity, e.g. one of at least 80%, 85%, 90%, 91%, 92%, 93%, 94%, 95%, 96%, 97%, 98% or 99% amino acid sequence identity to the amino acid sequence of SEQ ID NO: 7. In some embodiments, a polypeptide encoded by a SARS-CoV-2 may comprise or consist of the amino acid sequence of SEQ ID NO: 8, or an amino acid sequence having at least 70% sequence identity, e.g. one of at least 80%, 85%, 90%, 91%, 92%, 93%, 94%, 95%, 96%, 97%, 98% or 99% amino acid sequence identity to the amino acid sequence of SEQ ID NO: 8.

In some embodiments, a polypeptide encoded by a SARSr-CoV is selected from a spike protein, an envelope protein, a membrane protein and a nucleocapsid protein, or a fragment of a spike protein, envelope protein, membrane protein or nucleocapsid protein. In some embodiments, a polypeptide encoded by a SARSr-CoV is selected from a spike protein, an envelope protein or a membrane protein, or a fragment of a spike protein, envelope protein or membrane protein. In some embodiments, a polypeptide encoded by a SARSr-CoV is a spike protein or a fragment of a spike protein.

As used herein, a “fragment” of a reference protein/polypeptide may be of any length (by number of amino acids), although may optionally be at least 25% of the length of the reference protein/polypeptide and may have a maximum length of one of 50%, 75%, 80%, 85%, 90%, 91%, 92%, 93%, 94%, 95%, 96%, 97%, 98%, or 99% of the reference protein/polypeptide.

The SARSr-CoV spike protein comprises 51 and S2 subunits. The 51 subunit comprises a minimal receptor-binding domain (RBD) through which the SARSr-CoV binds to ACE2 expressed by host cells. Although virus neutralization can be achieved by other type of antibodies, receptor/entry blocking antibodies represent a substantial majority of neutralization antibodies for any given virus [33].

The RBD has been shown to be sufficient for binding to ACE2 in in vitro experiments [34]. From SARS studies, it has been shown that most neutralizing antibodies to SARS-CoV are directed against the RBD region [35].

The RBD in turn comprises the receptor binding motif (RBM), which is the region of the RBD that contacts ACE2.

In some embodiments, a polypeptide encoded by a SARSr-CoV may comprise or consist of the S1 subunit of a spike protein. In some embodiments, a polypeptide encoded by a SARSr-CoV may comprise or consist of the RBD of a spike protein. In some embodiments, a polypeptide encoded by a SARSr-CoV may comprise or consist of the RBM of a spike protein.

The S1 subunit of the spike protein of SARS-CoV may have the amino acid sequence shown in SEQ ID NO: 9. The RBD of the spike protein of SARS-CoV may have the amino acid sequence shown in SEQ ID NOs: 10 or 28. The RBM of the spike protein of SARS-CoV may have the amino acid sequence shown in SEQ ID NO: 11.

The S1 subunit of the spike protein of SARS-CoV-2 may have the amino acid sequence shown in SEQ ID NOs: 12 or 27. The RBD of the spike protein of SARS-CoV-2 may have the amino acid sequence shown in SEQ ID NOs: 13 or 26. The RBM of the spike protein of SARS-CoV-2 may have the amino acid sequence shown in SEQ ID NO: 14.

In some embodiments, a polypeptide encoded by a SARSr-CoV (e.g. a spike protein or a fragment thereof encoded by a SARSr-CoV) may comprise or consist of the amino acid sequence of SEQ ID NO: 9, or an amino acid sequence having at least 70% sequence identity, e.g. one of at least 80%, 85%, 90%, 91%, 92%, 93%, 94%, 95%, 96%, 97%, 98% or 99% amino acid sequence identity to the amino acid sequence of SEQ ID NO: 9. In some embodiments, a polypeptide encoded by a SARSr-CoV (e.g. a spike protein or a fragment thereof encoded by a SARSr-CoV) may comprise or consist of the amino acid sequence of SEQ ID NOs: 10 or 28, or an amino acid sequence having at least 70% sequence identity, e.g. one of at least 80%, 85%, 90%, 91%, 92%, 93%, 94%, 95%, 96%, 97%, 98% or 99% amino acid sequence identity to the amino acid sequence of SEQ ID NOs: 10 or 28. In some embodiments, a polypeptide encoded by a SARSr-CoV (e.g. a spike protein or a fragment thereof encoded by a SARSr-CoV) may comprise or consist of the amino acid sequence of SEQ ID NO: 11, or an amino acid sequence having at least 70% sequence identity, e.g. one of at least 80%, 85%, 90%, 91%, 92%, 93%, 94%, 95%, 96%, 97%, 98% or 99% amino acid sequence identity to the amino acid sequence of SEQ ID NO: 11.

In some embodiments, a polypeptide encoded by a SARSr-CoV (e.g. a spike protein or a fragment thereof encoded by a SARSr-CoV) may comprise or consist of the amino acid sequence of SEQ ID NOs: 12 or 27, or an amino acid sequence having at least 70% sequence identity, e.g. one of at least 80%, 85%, 90%, 91%, 92%, 93%, 94%, 95%, 96%, 97%, 98% or 99% amino acid sequence identity to the amino acid sequence of SEQ ID NOs: 12 or 27. In some embodiments, a polypeptide encoded by a SARSr-CoV (e.g. a spike protein or a fragment thereof encoded by a SARSr-CoV) may comprise or consist of the amino acid sequence of SEQ ID NOs: 13 or 26, or an amino acid sequence having at least 70% sequence identity, e.g. one of at least 80%, 85%, 90%, 91%, 92%, 93%, 94%, 95%, 96%, 97%, 98% or 99% amino acid sequence identity to the amino acid sequence of SEQ ID NOs: 13 or 26. In some embodiments, a polypeptide encoded by a SARSr-CoV (e.g. a spike protein or a fragment thereof encoded by a SARSr-CoV) may comprise or consist of the amino acid sequence of SEQ ID NO: 14, or an amino acid sequence having at least 70% sequence identity, e.g. one of at least 80%, 85%, 90%, 91%, 92%, 93%, 94%, 95%, 96%, 97%, 98% or 99% amino acid sequence identity to the amino acid sequence of SEQ ID NO: 14.

The present disclosure also concerns polypeptides which bind specifically to polypeptides/fragments thereof encoded by a SARSr-CoV.

As used herein, “specific binding” refers to interaction which is selective for the relevant molecule, and which can be discriminated from non-specific binding. A polypeptide that binds specifically to a given molecule preferably binds the molecule with greater affinity, and/or with greater duration than it binds to other molecules to which the polypeptide does not bind specifically.

The ability of a given polypeptide to bind specifically to a given molecule can be determined by analysis according to methods known in the art, such as by ELISA, Surface Plasmon Resonance (SPR; see e.g. Hearty et al., Methods Mol Biol (2012) 907:411-442), Bio-Layer Interferometry (see e.g. Lad et al., (2015) J Biomol Screen 20(4): 498-507), flow cytometry, or by a radiolabeled antigen-binding assay (MA) enzyme-linked immunosorbent assay. Through such analysis binding to a given molecule can be measured and quantified. In some embodiments, the binding may be the response detected in a given assay.

In some embodiments, the extent of binding of the polypeptide to a non-target molecule is less than about 10% of the level of binding to the molecule to which the polypeptide binds specifically, as measured e.g. by ELISA, SPR, Bio-Layer Interferometry or by RIA. Alternatively, specific binding may be reflected in terms of binding affinity, wherein the polypeptide binds to the molecule to which the polypeptide binds specifically with a dissociation constant (K_{D}) that is at least 0.1 order of magnitude (i.e. 0.1×10ⁿ, where n is an integer representing the order of magnitude) greater than the K_{D} of the polypeptide towards a non-target molecule. This may optionally be one of at least 0.2, 0.3, 0.4, 0.5, 0.6, 0.7, 0.8, 0.9, 1.0, 1.5, or 2.0.

A polypeptide which binds specifically to a polypeptide/fragment encoded by a SARSr-CoV may be e.g. an interaction partner for the polypeptide/fragment encoded by a SARSr-CoV, or an antigen-binding molecule which binds specifically to the polypeptide/fragment encoded by a SARSr-CoV.

An interaction partner for a polypeptide/fragment encoded by a SARSr-CoV may be any polypeptide which associates with the polypeptide/fragment. The association may involve covalent interaction (e.g. disulfide bonding) and/or non-covalent interaction (e.g. electrostatic interaction (e.g. ionic bonding, hydrogen bonding), Van der Waals forces) between the polypeptide/fragment encoded by a SARSr-CoV and the interaction partner.

As used herein, an “antigen-binding molecule” refers to a molecule which is capable of specific binding to a target polypeptide, and encompasses e.g. monoclonal antibodies, polyclonal antibodies, monospecific and multispecific antibodies (e.g., bispecific antibodies), and antibody fragments (e.g. Fv, scFv, Fab, scFab, F(ab′)2, Fab2, diabodies, triabodies, scFv-Fc, minibodies, single domain antibodies (e.g. VhH), etc.) and aptamers.

In some embodiments, an interaction partner for a polypeptide/fragment encoded by a SARSr-CoV is a polypeptide expressed by a cell to which the SARSr-CoV binds, or a fragment thereof. In some embodiments, an interaction partner is a receptor molecule expressed at the cell surface of a cell to which the SARSr-CoV binds, or a fragment thereof.

In some embodiments, a polypeptide which binds specifically to a polypeptide/fragment encoded by a SARSr-CoV may comprise or consist of the amino acid sequence of an ACE2 or a fragment thereof, or comprise or consist of an amino acid sequence having at least 60% sequence identity, e.g. one of at least 65%, 70%, 75%, 80%, 85%, 90%, 91%, 92%, 93%, 94%, 95%, 96%, 97%, 98%, 99% or 100% sequence identity to the amino acid sequence of an ACE2 or a fragment thereof.

Angiotensin-converting enzyme 2 (ACE2) is a single-pass type I transmembrane carboxypeptidase which attaches to the cell membrane of cells of the outer surface tissues of lungs, arteries, heart, kidney, and intestines. The structure and function of ACE2 is described e.g. in Hamming et al., J Pathol (2004) 203(2): 631-637, which is hereby incorporated by reference in its entirety. ACE2 has been identified to be the entry point into cells for SARS-CoV and SARS-CoV-2, via interaction with the spike protein. The SARSr-CoV spike protein binds to the extracellular domain of ACE2. ACE2 has been identified as the key cell entry receptor for both SARS-CoV and SARS-CoV-2 [3, 7].

In this specification “ACE2” refers to ACE2 from any species and includes ACE2 isoforms, fragments, variants (including mutants) or homologues from any species. In some embodiments, the ACE2 is ACE2 from a mammal (e.g. a therian, placental, epitherian, preptotheria, archontan, primate (rhesus, cynomolgous, non-human primate or human)). In some embodiments, the ACE2 is ACE2 from a human, bat, pangolin, civet or pig. Isoforms, fragments, variants or homologues of ACE2 may optionally be characterised as having at least 70% sequence identity, preferably one of 80%, 85%, 90%, 91%, 92%, 93%, 94%, 95%, 96%, 97%, 98%, 99% or 100% amino acid sequence identity to the amino acid sequence of an immature or mature ACE2 isoform from a given species, e.g. human.

Human ACE2 isoform 1's amino acid sequence is set forth in SEQ ID NOs: 15 or 29. Human ACE2 isoform 2's amino acid sequence is shown in SEQ ID NO: 16. The extracellular domain of human ACE2 isoform 1 is shown in SEQ ID NOs: 17 or 30.

The bat ACE2 may have the amino acid sequence shown in SEQ ID NOs: 18 or 31. The extracellular domain of bat ACE2 may have the amino acid sequence shown in SEQ ID NOs: 19 or 32.

Pangolin ACE2 may have the amino acid sequence shown in SEQ ID NO: 20. The extracellular domain of pangolin ACE2 may have the amino acid sequence shown in SEQ ID NO: 21.

Civet ACE2 may have the amino acid sequence shown in SEQ ID NO: 22. The extracellular domain of civet ACE2 may have the amino acid sequence shown in SEQ ID NO: 23.

Pig ACE2 may have the amino acid sequence shown in SEQ ID NOs: 24 or 33. The extracellular domain of pig ACE2 may have the amino acid sequence shown in SEQ ID NOs: 25 or 34.

In some embodiments, an ACE2 or fragment thereof may comprise or consist of the amino acid sequence of SEQ ID NOs: 15 or 29, or an amino acid sequence having at least 70% sequence identity, e.g. one of at least 80%, 85%, 90%, 91%, 92%, 93%, 94%, 95%, 96%, 97%, 98% or 99% amino acid sequence identity to the amino acid sequence of SEQ ID NOs: 15 or 29.

In some embodiments, an ACE2 or fragment thereof may comprise or consist of the amino acid sequence of SEQ ID NO: 16, or an amino acid sequence having at least 70% sequence identity, e.g. one of at least 80%, 85%, 90%, 91%, 92%, 93%, 94%, 95%, 96%, 97%, 98% or 99% amino acid sequence identity to the amino acid sequence of SEQ ID NO: 16.

In some embodiments, an ACE2 or fragment thereof may comprise or consist of the amino acid sequence of SEQ ID NOs: 17 or 30, or an amino acid sequence having at least 70% sequence identity, e.g. one of at least 80%, 85%, 90%, 91%, 92%, 93%, 94%, 95%, 96%, 97%, 98% or 99% amino acid sequence identity to the amino acid sequence of SEQ ID NOs: 17 or 30.

In some embodiments, an ACE2 or fragment thereof may comprise or consist of the amino acid sequence of SEQ ID NOs: 18 or 31, or an amino acid sequence having at least 70% sequence identity, e.g. one of at least 80%, 85%, 90%, 91%, 92%, 93%, 94%, 95%, 96%, 97%, 98% or 99% amino acid sequence identity to the amino acid sequence of SEQ ID NOs: 18 or 31.

In some embodiments, an ACE2 or fragment thereof may comprise or consist of the amino acid sequence of SEQ ID NOs: 19 or 32, or an amino acid sequence having at least 70% sequence identity, e.g. one of at least 80%, 85%, 90%, 91%, 92%, 93%, 94%, 95%, 96%, 97%, 98% or 99% amino acid sequence identity to the amino acid sequence of SEQ ID NOs: 19 or 32.

In some embodiments, an ACE2 or fragment thereof may comprise or consist of the amino acid sequence of SEQ ID NO: 20, or an amino acid sequence having at least 70% sequence identity, e.g. one of at least 80%, 85%, 90%, 91%, 92%, 93%, 94%, 95%, 96%, 97%, 98% or 99% amino acid sequence identity to the amino acid sequence of SEQ ID NO: 20.

In some embodiments, an ACE2 or fragment thereof may comprise or consist of the amino acid sequence of SEQ ID NO: 21, or an amino acid sequence having at least 70% sequence identity, e.g. one of at least 80%, 85%, 90%, 91%, 92%, 93%, 94%, 95%, 96%, 97%, 98% or 99% amino acid sequence identity to the amino acid sequence of SEQ ID NO: 21.

In some embodiments, an ACE2 or fragment thereof may comprise or consist of the amino acid sequence of SEQ ID NO: 22, or an amino acid sequence having at least 70% sequence identity, e.g. one of at least 80%, 85%, 90%, 91%, 92%, 93%, 94%, 95%, 96%, 97%, 98% or 99% amino acid sequence identity to the amino acid sequence of SEQ ID NO: 22.

In some embodiments, an ACE2 or fragment thereof may comprise or consist of the amino acid sequence of SEQ ID NO: 23, or an amino acid sequence having at least 70% sequence identity, e.g. one of at least 80%, 85%, 90%, 91%, 92%, 93%, 94%, 95%, 96%, 97%, 98% or 99% amino acid sequence identity to the amino acid sequence of SEQ ID NO: 23.

In some embodiments, an ACE2 or fragment thereof may comprise or consist of the amino acid sequence of SEQ ID NOs: 24 or 33, or an amino acid sequence having at least 70% sequence identity, e.g. one of at least 80%, 85%, 90%, 91%, 92%, 93%, 94%, 95%, 96%, 97%, 98% or 99% amino acid sequence identity to the amino acid sequence of SEQ ID NOs: 24 or 33.

In some embodiments, an ACE2 or fragment thereof may comprise or consist of the amino acid sequence of SEQ ID NOs: 25 or 34, or an amino acid sequence having at least 70% sequence identity, e.g. one of at least 80%, 85%, 90%, 91%, 92%, 93%, 94%, 95%, 96%, 97%, 98% or 99% amino acid sequence identity to the amino acid sequence of SEQ ID NOs: 25 or 34.

In some embodiments, the ACE2 or fragment according to the present disclosure is selected in accordance with the kits, compositions, uses and methods of the present disclosure to display (i) specific binding to the polypeptide/fragment thereof encoded by a SARSr-CoV (e.g. a spike protein or a fragment thereof encoded by a SARSr-CoV), and (ii) an affinity of binding to the polypeptide/fragment thereof encoded by a SARSr-CoV providing for sufficient sensitivity for the reliable detection of the presence in a sample of antibodies to the polypeptide/fragment thereof encoded by a SARSr-CoV (e.g. a spike protein or a fragment thereof encoded by a SARSr-CoV).

The present disclosure provides kits and compositions which may comprise a polypeptide encoded by a SARSr-CoV or a fragment thereof (e.g. a spike protein or a fragment thereof encoded by a SARSr-CoV), and/or a polypeptide which binds specifically to the polypeptide encoded by a SARSr-CoV or fragment (e.g. an ACE2 protein or a fragment thereof which binds specifically to a spike protein or a fragment thereof encoded by a SARSr-CoV).

In some embodiments in accordance with such aspects the kits and compositions may further comprise a solid support.

The solid support may be any solid support to which a polypeptide can readily be immobilised (e.g. by adsorption or conjugation), and which is suitable for the analysis of antibody containing samples, e.g. blood derived samples such as serum samples in accordance with the present disclosure. Suitable solid supports for use in such kits and compositions are well known to the skilled person.

In some embodiments, a solid support may comprise or consist of polystyrene, polypropylene, polycarbonate, cyclo-olefin, glass or quartz. In some embodiments, a solid support may be a microtiter (or “multiwell”) plate or microarray plate. In some embodiments, a solid support may be a bead, e.g. a magnetic bead. In some embodiments, a solid support may be a test strip, such as a lateral flow assay test strip, or a thin layer chromatography test strip. More particularly, a solid support may be a detection region/zone of a test strip. The test strip or the detection region of a test strip may be a porous membrane made of polysaccharides (e.g., cellulose materials such as paper and cellulose derivatives, such as cellulose acetate and nitrocellulose); polyether sulfone; polyethylene; nylon; polyvinylidene fluoride (PVDF); polyester; polypropylene; silica; inorganic materials, such as deactivated alumina, diatomaceous earth, MgSO₄, or other inorganic finely divided material uniformly dispersed in a porous polymer matrix, with polymers such as vinyl chloride, vinyl chloride-propylene copolymer, and vinyl chloride-vinyl acetate copolymer; cloth, both naturally occurring (e.g., cotton) and synthetic (e.g., nylon or rayon); porous gels, such as silica gel, agarose, dextran, and gelatin; polymeric films, such as polyacrylamide; and the like. In some embodiments, the test strip or the detection region of a test strip is made of nitrocellulose or cellulose acetate.

A polypeptide according to the present disclosure may be immobilised on (or ‘coated’ on) a solid support according to the present disclosure in accordance with methods well known to the skilled person. A polypeptide may be covalently or non-covalently immobilised on a solid support.

For example, a polypeptide may be immobilised on a solid support by applying a solution of a polypeptide in buffer solution under conditions suitable for, and for sufficient time to allow, the polypeptide to adsorb to the surface of the solid support. Alternatively, a polypeptide may be conjugated to a solid support, e.g. through a covalent bond. A polypeptide may be immobilized on a solid support via e.g., Van der Waals forces, chemical electronic bonds formed between the chemical group(s) on the polypeptide and the chemical group(s) on the solid support, and the like.

In some embodiments, articles of the present disclosure may further comprise means for detecting interaction between a polypeptide encoded by a SARSr-CoV or a fragment thereof (e.g. a spike protein or a fragment thereof encoded by a SARSr-CoV) and a polypeptide which binds specifically to the polypeptide encoded by the SARSr-CoV or fragment (e.g. an ACE2 protein or a fragment thereof which binds specifically to a spike protein or a fragment thereof encoded by the SARSr-CoV).

The means for detecting interaction can be any suitable means. For example, the means could employ an antibody capable of specifically binding to the polypeptide complex formed by interaction between the polypeptide encoded by a SARSr-CoV or a fragment thereof and the polypeptide which binds specifically to the polypeptide encoded by the SARSr-CoV or fragment, or could employ a reporter of interaction between the polypeptide encoded by a SARSr-CoV or a fragment thereof and the polypeptide which binds specifically to the polypeptide encoded by the SARSr-CoV or fragment.

In some embodiments, the means for detecting interaction may employ a detection entity. By way of illustration, in the experimental examples hereinbelow SARS-CoV-2 S1 and RBD polypeptides are conjugated to a horseradish peroxidase (HRP) moiety. After washing to remove unbound SARS-CoV-2 S1 and RBD polypeptide, the level of horseradish peroxidase activity is indicative of the amount of S1/RBD bound to the immobilised ACE2.

Accordingly, in some embodiments in accordance with the various aspects of the present disclosure the polypeptide encoded by a SARSr-CoV or a fragment thereof (e.g. a spike protein or a fragment thereof encoded by a SARSr-CoV), and/or the polypeptide which binds specifically to the polypeptide encoded by a SARSr-CoV or fragment (e.g. an ACE2 protein or a fragment thereof which binds specifically to a spike protein or a fragment thereof encoded by a SARSr-CoV) are/is conjugated to a detection entity for use to detect interaction between the polypeptide encoded by a SARSr-CoV or a fragment thereof and the polypeptide which binds specifically to the polypeptide encoded by the SARSr-CoV or fragment.

The polypeptide encoded by a SARSr-CoV or a fragment thereof (e.g. a spike protein or a fragment thereof encoded by a SARSr-CoV), and/or the polypeptide which binds specifically to the polypeptide encoded by a SARSr-CoV or fragment may be conjugated to a) a detection entity, or b) a biotin molecule which binds or is able to bind an avidin or streptavidin-conjugated detection entity.

A detection entity may, for example, be a detectable moiety, e.g. a fluorescent, luminescent, immuno-detectable, radio, chemical, nucleic acid or polypeptide label. In some embodiments, the fluorescent label is fluorescein isothiocyanate or phycoerythrin. In some embodiments, the luminescent label is colloidal gold, or colloidal silver. In some embodiments, a detection entity may be a moiety having detectable activity, e.g. an enzymatic activity on a given substrate. Examples of detection entities having detectable activity include e.g. horseradish peroxidase (HRP) and luciferase moieties.

In some embodiments, the detection entity is a colloidal gold nanoparticle, a colloidal silver nanoparticle, a fluorescent microsphere, or a quantum dot.

In some embodiments wherein a detection entity is a moiety having detectable activity, the kit or composition according to the present disclosure may further comprise reagents required for analysis of the detectable activity.

In some embodiments, the kit or composition according to the disclosure may employ detection by chemiluminescence. That is, in some embodiments, the detectable activity is chemiluminescence. Assays based on detection of chemiluminescence are described e.g. in Kricka et al., Analytica chimica acta, (2003), 500(1): 279-286 and Chen et al., Chinese Journal of Analytical Chemistry (2012) 40(1): 3-10, both of which are hereby incorporated by reference in their entirety.

In some embodiments a detection entity may e.g. be a chemical entity which produces electromagnetic radiation upon appropriate excitation. In some embodiments, a chemical entity may be an acridinium compound (e.g. an acridinium ester or acridinium sulfonamide ester), which may be excited using alkaline hydrogen peroxide.

In some embodiments a detection entity may e.g. be an enzyme entity which catalyses the production of electromagnetic radiation from a luminescent chemical. In some embodiments, an enzyme entity may be HRP, which may be used to catalyse decomposition of luminol in the presence of hydrogen peroxide. In some embodiments, an enzyme entity may be alkaline phosphatase, which may be used to catalyse decomposition of AMPPD.

In some embodiments, the polypeptide encoded by a SARSr-CoV or a fragment thereof (e.g. a spike protein or a fragment thereof (e.g. RBD) encoded by a SARSr-CoV) is provided in the kit or composition of the present disclosure such that in use to analyse a sample, the quantity or concentration of the polypeptide encoded by a SARSr-CoV or a fragment thereof (e.g. a spike protein or a fragment thereof (e.g. RBD) encoded by a SARSr-CoV) is (a) less than or equal to (in molar ratio) the quantity/concentration of neutralising antibodies to the SARSr-CoV in the sample being analysed using the kit or composition, and/or (b) sufficient to produce a detectable signal of interaction between the polypeptide encoded by a SARSr-CoV or a fragment thereof (e.g. a spike protein or a fragment thereof (e.g. RBD) encoded by a SARSr-CoV) and a polypeptide which binds specifically to the polypeptide encoded by the SARSr-CoV or fragment (e.g. an ACE2 protein or a fragment thereof which binds specifically to a spike protein or a fragment thereof encoded by the SARSr-CoV) in the absence of neutralising antibodies to the SARSr-CoV in the sample being analysed using the kit or composition.

The skilled person is able to determine suitable quantities/concentrations of polypeptide encoded by a SARSr-CoV (e.g. a spike protein) or fragment thereof where the compositions and kits are put to such use e.g. by determination of/reference to the quantity/concentration of neutralising antibodies to the SARSr-CoV in samples determined or otherwise known to contain neutralising antibodies to the SARSr-CoV. For example, in some embodiments a suitable quantity/concentration of the polypeptide may be less than or equal to (in molar ratio) the average (e.g. the mean) quantity/concentration of neutralising antibodies to the SARSr-CoV in reference sample(s) containing neutralising antibodies to the SARSr-CoV. In some embodiments, serial dilution of the sample to be tested may be used to determine a suitable quantity of a polypeptide encoded by a SARSr-CoV (e.g. a spike protein) or fragment thereof.

The skilled person is also able to determine suitable quantities/concentrations of polypeptide encoded by a SARSr-CoV (e.g. a spike protein) or fragment thereof where the compositions and kits are put to such use e.g. by determination of/reference to the minimal quantity/concentration required to produce a detectable signal of interaction between the polypeptide encoded by a SARSr-CoV or a fragment thereof (e.g. a spike protein or a fragment thereof (e.g. RBD) encoded by a SARSr-CoV) and the polypeptide which binds specifically to the polypeptide encoded by a SARSr-CoV or fragment (e.g. ACE2) in the absence of neutralising antibodies to the SARSr-CoV in the sample.

The present disclosure also provides the constituent polypeptides of the kits and compositions described herein. The polypeptides may be provided in isolated or substantially purified form.

In some embodiments the polypeptides of the present disclosure may comprise one or more linker sequences between amino acid sequences, e.g. between the amino acid sequence of a spike protein or a fragment thereof encoded by a SARSr-CoV and a detection entity.

Linker sequences are known to the skilled person, and are described, for example in Chen et al., Adv Drug Deliv Rev (2013) 65(10): 1357-1369, which is hereby incorporated by reference in its entirety. In some embodiments, a linker sequence may be a flexible linker sequence. Flexible linker sequences allow for relative movement of the amino acid sequences which are linked by the linker sequence. Flexible linkers are known to the skilled person, and several are identified in Chen et al., Adv Drug Deliv Rev (2013) 65(10): 1357-1369. Flexible linker sequences often comprise high proportions of glycine and/or serine residues.

In some embodiments, the linker sequence may comprise at least one glycine residue and/or at least one serine residue. In some embodiments the linker sequence may consist of glycine and serine residues. In some embodiments, the linker sequence may have a length of 1-2, 1-3, 1-4, 1-5 or 1-10 amino acids.

The polypeptides of the present disclosure may additionally comprise further amino acids or sequences of amino acids. For example, the polypeptide may comprise amino acid sequence(s) to facilitate expression, folding, trafficking, processing or purification of the polypeptide. For example, the polypeptide may comprise a sequence encoding a His, (e.g. 6×His), Myc, GST, MBP, FLAG, HA, E, or Biotin tag, optionally at the N- or C-terminus of the polypeptide.

The polypeptides of the present disclosure may additionally comprise a signal peptide (also known as a leader sequence or signal sequence). Signal peptides normally consist of a sequence of 5-30 hydrophobic amino acids, which form a single alpha helix. Secreted proteins and proteins expressed at the cell surface often comprise signal peptides.

The signal peptide may be present at the N-terminus of the polypeptide, and may be present in the newly synthesised polypeptide. The signal peptide provides for efficient trafficking and secretion of the polypeptide. Signal peptides are often removed by cleavage, and thus are not comprised in the mature polypeptide secreted from the cell expressing the polypeptide.

Signal peptides are known for many proteins, and are recorded in databases such as GenBank, UniProt, Swiss-Prot, TrEMBL, Protein Information Resource, Protein Data Bank, Ensembl, and InterPro, and/or can be identified/predicted e.g. using amino acid sequence analysis tools such as SignalP (Petersen et al., 2011 Nature Methods 8: 785-786) or Signal-BLAST (Frank and Sippl, 2008 Bioinformatics 24: 2172-2176).

Polypeptides according to the disclosure may be prepared according to methods for the production of polypeptides known to the skilled person.

Polypeptides may be prepared by chemical synthesis, e.g. liquid or solid phase synthesis. For example, peptides/polypeptides can by synthesised using the methods described in, for example, Chandrudu et al., Molecules (2013), 18: 4373-4388, which is hereby incorporated by reference in its entirety.

Alternatively, polypeptides may be produced by recombinant expression. Molecular biology techniques suitable for recombinant production of polypeptides are well known in the art, such as those set out in Green and Sambrook, Molecular Cloning: A Laboratory Manual (4th Edition), Cold Spring Harbor Press, 2012, and in Nat Methods. (2008); 5(2): 135-146, both of which are hereby incorporated by reference in their entirety.

For recombinant production according to the disclosure, any cell suitable for the expression of polypeptides may be used. The cell may be a prokaryote or eukaryote. In some embodiments the cell is a prokaryotic cell, such as a cell of archaea or bacteria. In some embodiments the bacteria may be Gram-negative bacteria such as bacteria of the family Enterobacteriaceae, for example Escherichia coli. In some embodiments, the cell is a eukaryotic cell such as a yeast cell, a plant cell, insect cell or a mammalian cell, e.g. CHO, HEK (e.g. HEK293), HeLa or COS cells. In some embodiments, the cell is a CHO cell that transiently or stably expresses the polypeptides.

In some cases the cell is not a prokaryotic cell because some prokaryotic cells do not allow for the same folding or post-translational modifications as eukaryotic cells. In addition, very high expression levels are possible in eukaryotes and proteins can be easier to purify from eukaryotes using appropriate tags. Specific plasmids may also be utilised which enhance secretion of the protein into the media.

In some embodiments polypeptides may be prepared by cell-free-protein synthesis (CFPS), e.g. using a system described in Zemella et al. Chembiochem (2015) 16(17): 2420-2431, which is hereby incorporated by reference in its entirety.

Production may involve culture or fermentation of a eukaryotic cell modified to express the polypeptide of interest. The culture or fermentation may be performed in a bioreactor provided with an appropriate supply of nutrients, air/oxygen and/or growth factors. Secreted proteins can be collected by partitioning culture media/fermentation broth from the cells, extracting the protein content, and separating individual proteins to isolate secreted polypeptide. Culture, fermentation and separation techniques are well known to those of skill in the art, and are described, for example, in Green and Sambrook, Molecular Cloning: A Laboratory Manual (4th Edition; incorporated by reference herein above).

Bioreactors include one or more vessels in which cells may be cultured. Culture in the bioreactor may occur continuously, with a continuous flow of reactants into, and a continuous flow of cultured cells from, the reactor. Alternatively, the culture may occur in batches. The bioreactor monitors and controls environmental conditions such as pH, oxygen, flow rates into and out of, and agitation within the vessel such that optimum conditions are provided for the cells being cultured.

Following culturing the cells that express the polypeptide, the polypeptide of interest may be isolated. Any suitable method for separating proteins from cells known in the art may be used. In order to isolate the polypeptide it may be necessary to separate the cells from nutrient medium. If the polypeptide is secreted from the cells, the cells may be separated by centrifugation from the culture media that contain the secreted polypeptide of interest. If the polypeptide of interest is collected within the cell, protein isolation may comprise centrifugation to separate cells from cell culture medium, treatment of the cell pellet with a lysis buffer, and cell disruption e.g. by sonification, rapid freeze-thaw or osmotic lysis.

It may then be desirable to isolate the polypeptide of interest from the supernatant or culture medium, which may contain other protein and non-protein components. A common approach to separating protein components from a supernatant or culture medium is by precipitation. Proteins of different solubilities are precipitated at different concentrations of precipitating agent such as ammonium sulfate. For example, at low concentrations of precipitating agent, water soluble proteins are extracted. Thus, by adding different increasing concentrations of precipitating agent, proteins of different solubilities may be distinguished. Dialysis may be subsequently used to remove ammonium sulfate from the separated proteins.

Other methods for distinguishing different proteins are known in the art, for example ion exchange chromatography and size chromatography. These may be used as an alternative to precipitation, or may be performed subsequently to precipitation.

Once the polypeptide of interest has been isolated from culture it may be desired or necessary to concentrate the polypeptide. A number of methods for concentrating proteins are known in the art, such as ultrafiltration or lyophilisation.

The present disclosure provides a nucleic acid encoding a polypeptide according to the present disclosure. It will be appreciated that “a nucleic acid” encompasses a plurality of such nucleic acids.

In some embodiments, the nucleic acid is purified or isolated, e.g. from other nucleic acid, or naturally-occurring biological material. In some embodiments the nucleic acid(s) comprise or consist of DNA and/or RNA.

The present disclosure also provides a vector which may comprise the nucleic acid according to the present disclosure.

The nucleotide sequence may be contained in a vector, e.g. an expression vector. A “vector” as used herein is a nucleic acid molecule used as a vehicle to transfer exogenous nucleic acid into a cell. The vector may be a vector for expression of the nucleic acid in the cell. Such vectors may include a promoter sequence operably linked to the nucleotide sequence encoding the sequence to be expressed. A vector may also include a termination codon and expression enhancers. Any suitable vectors, promoters, enhancers and termination codons known in the art may be used to express a peptide or polypeptide from a vector according to the disclosure.

The term “operably linked” may include the situation where a selected nucleic acid sequence and a regulatory nucleic acid sequence (e.g. promoter and/or enhancer) are covalently linked in such a way as to place the expression of nucleic acid sequence under the influence or control of the regulatory sequence (thereby forming an expression cassette). Thus a regulatory sequence is operably linked to the selected nucleic acid sequence if the regulatory sequence is capable of effecting transcription of the nucleic acid sequence. The resulting transcript(s) may then be translated into a desired peptide(s)/polypeptide(s).

Suitable vectors include plasmids, binary vectors, DNA vectors, mRNA vectors, viral vectors (e.g. gammaretroviral vectors (e.g. murine Leukemia virus (MLV)-derived vectors), lentiviral vectors, adenovirus vectors, adeno-associated virus vectors, vaccinia virus vectors and herpesvirus vectors), transposon-based vectors, and artificial chromosomes (e.g. yeast artificial chromosomes).

In some embodiments, the vector may be a eukaryotic vector, e.g. a vector which may comprises the elements necessary for expression of protein from the vector in a eukaryotic cell. In some embodiments, the vector may be a mammalian vector, e.g. that may comprises a cytomegalovirus (CMV) or SV40 promoter to drive protein expression.

The present disclosure also provides a cell which may comprises or expresses a polypeptide according to the present disclosure. Also provided is a cell which may comprises or expresses a nucleic acid or vector according to the present disclosure. The cell may be a eukaryotic cell, e.g. a mammalian cell. The mammal may be a primate (rhesus, cynomolgous, non-human primate or human) or a non-human mammal (e.g. rabbit, guinea pig, rat, mouse or other rodent (including any animal in the order Rodentia), cat, dog, pig, sheep, goat, cattle (including cows, e.g. dairy cows, or any animal in the genus Bos), horse (including any animal in the family Equidae), donkey, and non-human primate).

The present disclosure also provides a method for producing a cell which may comprises a nucleic acid or vector according to the present disclosure, which may comprise introducing a nucleic acid or vector according to the present disclosure into a cell. In some embodiments, introducing an isolated nucleic acid or vector according to the present disclosure into a cell comprises transformation, transfection, electroporation or transduction (e.g. retroviral transduction).

The present disclosure also provides a method for producing a cell which may express or comprise a polypeptide according to the present disclosure, which may comprise introducing a nucleic acid or vector according to the present disclosure in a cell. In some embodiments, the methods additionally comprise culturing the cell under conditions suitable for expression of the nucleic acid or vector by the cell. In some embodiments, the methods are performed in vitro.

The present disclosure also provides cells obtained or obtainable by the methods according to the present disclosure.

The present disclosure also concerns methods using and uses of the kits, compositions and polypeptides of the present disclosure.

The articles of the present disclosure are useful for detecting antibodies to a polypeptide encoded by a SARSr-CoV or a fragment thereof (e.g. a spike protein or a fragment thereof encoded by a SARSr-CoV).

In particular, the articles of the present disclosure are useful for detecting the presence of antibodies which reduce or inhibit binding of a polypeptide encoded by a SARSr-CoV or fragment thereof to a polypeptide which binds specifically to the polypeptide encoded by a SARSr-CoV or fragment. Such antibodies may be referred to as neutralising antibodies.

By way of further explanation, with reference to the experimental examples hereinbelow, the presence of antibodies which inhibit binding of SARS-CoV-2 spike protein S1 subunit and SARS-CoV-2 spike protein RBD are detected in samples based on the determination of a reduced level of interaction between the SARS-CoV-2 spike protein S1 subunit or SARS-CoV-2 spike protein RBD with ACE2 immobilised on the solid support. The presence of neutralising antibodies in the sample is inferred from the determination of a reduced level of interaction relative to a control condition lacking neutralising antibodies to SARS-CoV-2 spike protein S1 subunit or SARS-CoV-2 spike protein RBD.

Accordingly, the articles of the present disclosure are useful in methods for detecting the presence of antibodies to a SARSr-CoV in a sample. It will be appreciated that the methods are useful for the detection of the presence of antibodies to the spike protein or a fragment thereof encoded by a SARSr-CoV employed in the kit/composition.

By way of illustration, the assays exemplified herein employ SARS-CoV-2 spike protein S1 subunit or SARS-CoV-2 spike protein RBD, and are therefore useful for the detection of antibodies to SARS-CoV-2 spike protein S1 subunit or SARS-CoV-2 spike protein RBD.

Detection of the presence of antibodies to a SARSr-CoV in a sample may be indicative of an ongoing or previous infection with the SARSr-CoV. Detection of the presence of antibodies to a SARSr-CoV in a sample may be indicative of an ongoing or previous immune response, particularly a humoral immune response, to the SARSr-CoV.

Accordingly, the present disclosure provides methods for determining whether a subject is or has been infected with a SARSr-CoV, and also provides methods for determining whether a subject is mounting/has mounted an immune response (e.g. a humoral immune response) to a SARSr-CoV.

The methods may generally comprise analysing a sample in order to determine whether its contents reduce or inhibit interaction between a polypeptide encoded by a SARSr-CoV (e.g. a spike protein) or fragment thereof and a polypeptide which binds specifically to the polypeptide encoded by a SARSr-CoV or fragment (e.g. ACE2), relative to the level of interaction observed for an appropriate negative control condition.

An appropriate negative control condition may e.g. employ an equivalent sample known to lack antibodies capable of reducing or inhibiting interaction between a polypeptide encoded by a SARSr-CoV (e.g. a spike protein) or fragment thereof and a polypeptide which binds specifically to the polypeptide encoded by a SARSr-CoV or fragment (e.g. ACE2). For example, a control sample may be from a known naïve subject, e.g. a subject known not to have been infected with the SARSr-CoV.

Determination of reduced or inhibited interaction indicates the presence of neutralising antibodies to the polypeptide encoded by a SARSr-CoV or fragment thereof (e.g. the spike protein or fragment thereof) in the sample.

As used herein, “reduced” or “inhibited” interaction may be a level of interaction which is less than 1 times, e.g. <0.99 times, <0.95 times, <0.9 times, <0.85 times, <0.8 times, <0.75 times, <0.7 times, <0.65 times, <0.6 times, <0.55 times, <0.5 times, <0.45 times, <0.4 times, <0.35 times, <0.3 times, <0.25 times, <0.2 times, <0.15 times, <0.1 times, <0.05 times, or <0.01 times the level of interaction between the polypeptide encoded by a SARSr-CoV (e.g. a spike protein) or fragment thereof and the polypeptide which binds specifically to the polypeptide encoded by a SARSr-CoV or fragment (e.g. ACE2) observed in the negative control condition.

Alternatively, “reduced” or “inhibited” interaction may be expressed in terms of percentage inhibition of the level of interaction between the polypeptide encoded by a SARSr-CoV (e.g. a spike protein) or fragment thereof and the polypeptide which binds specifically to the polypeptide encoded by a SARSr-CoV or fragment (e.g. ACE2) observed in the negative control condition. In such instances, “reduced” or “inhibited” interaction may refer to percentage inhibition of greater than 0%, e.g. greater than 5%, 10%, 15%, 20%, 25%, 30%, 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%, or 100% of the level of interaction between the polypeptide encoded by a SARSr-CoV (e.g. a spike protein) or fragment thereof and the polypeptide which binds specifically to the polypeptide encoded by a SARSr-CoV or fragment (e.g. ACE2) observed in the negative control condition.

In some embodiments, aspects of the present disclosure may comprise:
contacting a sample obtained from the subject with: (i) a polypeptide encoded by a SARSr-CoV (e.g. a spike protein) or fragment thereof, and (ii) a polypeptide which binds specifically to the polypeptide encoded by a SARSr-CoV or fragment (e.g. ACE2), and
determining the level of interaction between the polypeptide or fragment of (i) and the polypeptide of (ii).

In some embodiments, aspects of the present disclosure may comprise:
(a) contacting a sample obtained from the subject with (i) a polypeptide encoded by a SARSr-CoV (e.g. a spike protein) or fragment thereof,
(b) contacting the mixture formed by step (a) with (ii) a polypeptide which binds specifically to the polypeptide encoded by a SARSr-CoV or fragment (e.g. ACE2), and
(c) determining the level of interaction between the polypeptide or fragment of (i) and the polypeptide of (ii).

In some embodiments, aspects of the present disclosure may comprise:
contacting the sample with the spike protein or fragment of (i),
immobilizing the spike protein or fragment of (i) to the solid support,
contacting the spike protein or fragment of (i) immobilized on the solid support with the ACE2 or fragment of (ii), and
determining the level of interaction between the spike protein or fragment of (i) and the ACE2 or fragment of (ii),
   wherein the ACE2 or fragment of (ii) is conjugated to the detection entity, and
   (a) the spike protein or fragment of (i) is conjugated to a biotin molecule, the solid support is a microtiter plate or a bead, and the solid support is conjugated with an avidin or a streptavidin, or
   (b) the spike protein or fragment of (i) is conjugated to an avidin or a streptavidin, the solid support is a bead, and the solid support is conjugated with a biotin.

The method may comprise the step of removing components that do not bind the spike protein or fragment of (i), prior to the step of contacting the spike protein or fragment of (i) immobilized on the solid support with the ACE2 or fragment of (ii).

In some embodiments, aspects of the present disclosure may comprise:
immobilizing the spike protein or fragment of (i) to the solid support,
contacting the sample with the spike protein or fragment of (i) immobilized on the solid support,
contacting the spike protein or fragment of (i) immobilized on the solid support with the ACE2 or fragment of (ii), and
determining the level of interaction between the spike protein or fragment of (i) and the ACE2 or fragment of (ii),
   wherein the solid support is a bead or a microtiter plate, and the ACE2 or fragment of (ii) is conjugated to a) the detection entity orb) the biotin molecule which binds an avidin or streptavidin-conjugated detection entity.

The method may comprise the step of removing components that do not bind the spike protein or fragment of (i), after the step of contacting the sample with the spike protein or fragment of (i) immobilized on the solid support.

In some embodiments, the solid support is a bead, e.g., a magnetic bead. The quantity of the spike protein or fragment of (i) immobilized on the solid support may be regulated by adjusting a) the quantity of the spike protein or fragment of (i) incubated with the bead, b) the quantity of the bead incubated with the spike protein or fragment of (i), and/or (c) the space, e.g., the surface area, of a bead for attaching/conjugating the spike protein or fragment of (i). The molar ratio of the spike protein or fragment of (i) to the antibodies to a SARSr-CoV in the sample may be optimized by e.g., adjusting the spike protein or fragment of (i) immobilized on the bead, such that the method of the disclosure may detect antibodies with higher sensitivity and/or higher specificity.

In some embodiments, aspects of the present disclosure may comprise:
(a) contacting a sample obtained from the subject with (i) a polypeptide which binds specifically to the polypeptide encoded by a SARSr-CoV or fragment (e.g. ACE2),
(b) contacting the mixture formed by step (a) with (ii) a polypeptide encoded by a SARSr-CoV (e.g. a spike protein) or fragment thereof, and
(c) determining the level of interaction between the polypeptide or fragment of (i) and the polypeptide of (ii).

In some embodiments, “contacting” samples/mixtures with polypeptides according to the present disclosure may comprise mixing such samples/mixture and compositions (e.g. solutions) which may comprise the polypeptides, e.g. in an appropriate container or on an appropriate solid support.

In some embodiments, “contacting” samples/mixtures with polypeptides according to the present disclosure may comprise applying a sample/mixture to a solid support to which the polypeptides are immobilised.

In some embodiments, the aspects may comprise contacting the sample with a composition which comprises a polypeptide encoded by a SARSr-CoV (e.g. a spike protein) or fragment thereof (RBD)), wherein the polypeptide is provided at a quantity or concentration which is (a) less than or equal to (in terms of molar ratio) the quantity/concentration of neutralising antibodies to the SARSr-CoV in the sample, and/or (b) sufficient to produce a detectable signal of interaction between the polypeptide or fragment of (i) and the polypeptide or fragment of (ii) in the absence of neutralising antibodies to the SARSr-CoV in the sample.

In some embodiments, the aspects may comprise determining the presence or absence of neutralising antibodies to the SARSr-CoV in the sample. In some embodiments, the aspects may comprise determining the quantity and/or concentration of neutralising antibodies to the SARSr-CoV in the sample.

The skilled person is able to determine suitable quantities/concentrations of polypeptide encoded by a SARSr-CoV (e.g. a spike protein) or fragment thereof to be employed in such methods e.g. by determination of/reference to the quantity/concentration of neutralising antibodies to the SARSr-CoV in samples determined or otherwise known to contain neutralising antibodies to the SARSr-CoV. For example, in some embodiments a suitable quantity/concentration of the polypeptide may be less than or equal to (in molar ratio) the average (e.g. the mean) quantity/concentration of neutralising antibodies to the SARSr-CoV in reference sample(s) containing neutralising antibodies to the SARSr-CoV.

The skilled person is also able to determine suitable quantities/concentrations of polypeptide encoded by a SARSr-CoV (e.g. a spike protein) or fragment thereof to be employed in such methods e.g. by determination of/reference to the minimal quantity/concentration required to produce a detectable signal of interaction between the polypeptide encoded by a SARSr-CoV or a fragment thereof (e.g. a spike protein or a fragment thereof (e.g. RBD) encoded by a SARSr-CoV) and the polypeptide which binds specifically to the polypeptide encoded by a SARSr-CoV or fragment (e.g. ACE2) in the absence of neutralising antibodies to the SARSr-CoV in the sample.

In some embodiments, the aspects may employ a range of different quantities/concentrations of polypeptide encoded by a SARSr-CoV (e.g. a spike protein) or fragment thereof for the analysis of a given sample. That is, in some embodiments, aspects of the present disclosure may comprise performing the analysis with a range of different quantities/concentrations of the polypeptide encoded by a SARSr-CoV (e.g. a spike protein) or fragment thereof for the analysis of aliquots of a given same sample. The range of different quantities/concentrations of the polypeptide may be a dilution series, and may e.g. by prepared by serial dilution of a solution of the polypeptide.

In some embodiments, the aspects may further comprise one or more of the following:
obtaining a sample (e.g. a blood sample) from a subject;
preparing a blood-derived sample (e.g. a serum sample) from a blood sample obtained from a subject;
providing a composition or kit according to the present disclosure;
applying a blood-derived sample (e.g. a serum sample) to a composition according to the present disclosure;
incubating the sample with the polypeptides/composition for sufficient time and under appropriate conditions for the formation of antibody:antigen complexes;
aspiration of the sample;
washing to remove unbound proteins;
detection of interaction between the polypeptide or fragment of (i) and the polypeptide or fragment of (ii).

In some embodiments, the aspects may further comprise determining the level (e.g. the percentage) of inhibition of interaction between the polypeptide or fragment of (i) and the polypeptide or fragment of (ii).

In some embodiments, the aspects may further comprise comparing the level (e.g. the percentage) of inhibition of interaction between the polypeptide or fragment of (i) and the polypeptide or fragment of (ii) as observed to a reference threshold value for determining that the sample contains neutralising antibodies to the SARSr-CoV. In some embodiments, the aspects may further comprise determining on the basis of such comparison whether a sample contains or does not contain neutralising antibodies to the SARSr-CoV (i.e. determining the presence or absence of neutralising antibodies to the SARSr-CoV in the sample).

Appropriate reagents, buffers and washing steps to be employed in such methods are well known to the person skilled in the art of molecular biology, and can moreover be inferred by reference to the experimental examples herein and Bossart et al., J Viol Meth (2007) 142: 29-40, which is hereby incorporated by reference in its entirety.

In some aspects, the articles and methods of the present disclosure are employed for the diagnosis of infection with a SARSr-CoV, e.g. SARS-CoV-2. In some aspects, the articles and methods are employed for the diagnosis of a disease caused by infection with a SARSr-CoV, e.g. COVID-19.

Aspects of the present disclosure concern the analysis of samples from subjects. The sample may be of, or may be derived from, any product produced by a subject. A sample may be taken from any tissue or bodily fluid, e.g. a blood sample (including blood-derived samples), serum sample, lymph sample, saliva sample, synovial fluid sample. A blood-derived sample may be a selected fraction of a patient's blood, e.g. a selected cell-containing fraction, or a plasma or serum fraction.

The sample may be any sample containing the antibody products of a humoral immune response. In some embodiments the sample obtained from a subject is an antibody-containing sample or antibody-containing fraction thereof. The sample is preferably a blood sample or a blood-derived sample, more preferably a serum sample.

Samples may be collected (e.g. by venesection), processed and/or stored (e.g. frozen and stored at −80° C.) according to known techniques, and in accordance with the analysis to be subsequently performed on the sample.

A sample may be or may have been collected from a subject of interest. The subject according to the various aspects of the present disclosure may be any animal. In some embodiments, the subject is a mammal (e.g. a therian, placental, epitherian, preptotheria, archontan, primate (rhesus, cynomolgous, non-human primate or human)). In some embodiments, the subject is a human, bat, pangolin, civet or pig. The subject may be a non-human mammal, but is more preferably human. The subject may be male or female. The subject may be a patient.

The subject may be suspected of being infected with a SARSr-CoV, or may be suspected of having a disease caused by infection with a SARSr-CoV. The subject may be suspected of having previously been infected with a SARSr-CoV, or may be suspected of having previously had a disease caused by infection with a SARSr-CoV.

The subject may have been diagnosed with infection with a SARSr-CoV or may have been diagnosed with a disease caused by infection with a SARSr-CoV. The subject may have experienced infection with a SARSr-CoV or may have had a disease caused by infection with a SARSr-CoV.

In particular embodiments, the articles and methods of the present disclosure provide for the detection of the presence in a sample (e.g. a patient-derived sample) of neutralising antibodies to a SARSr-CoV (e.g. SARS-CoV-2), based on detection of blocking of the interaction/binding between an ACE2 protein or fragment thereof and a spike protein or fragment thereof encoded by the SARSr-CoV (e.g. the RBD of the SARSr-CoV) by such neutralising antibodies.

More particularly, preferred embodiments provide for the detection of the presence in a sample (e.g. a sample derived from a COVID-19 patient) of neutralising antibodies to a SARS-CoV-2, based on detection of blocking of the interaction/binding between an ACE2 protein or fragment thereof and the RBD of the SARS-CoV-2 by such neutralising antibodies.

Even where such neutralising antibodies are present in a sample, they will often be present at low levels (i.e. there will be a limited quantity of the antibody). In such instances, if there is an over-supply (i.e. an excess) of spike protein/fragment thereof (e.g. RBD) in the reaction mix, the quantity of antibodies in a test sample may be insufficient to bind to all of the spike protein/fragment thereof (e.g. RBD) available, and the surplus spike protein/fragment thereof (e.g. RBD) will bind to the ACE2 protein/fragment thereof yielding signal, and thus resulting in a false-negative result.

Therefore, in preferred embodiments in accordance with the preset disclosure, the quantity of spike protein/fragment thereof (e.g. RBD) is present in the reaction mix at a level which is (i) sufficient to provide a detectable level of signal (i.e. on interaction with the ACE2 protein/fragment thereof) in the absence of neutralising antibodies to the SARSr-CoV to enable the correct determination of true negative samples lacking neutralising antibodies to the SARSr-CoV, but (ii) not more, in terms of molar ratio (i.e. no molar excess of), than the amount of neutralising antibodies to the SARSr-CoV in the sample, such that all of the spike protein/fragment thereof (e.g. RBD) present in the reaction mix will be neutralized by the antibodies available in the sample, and false-negative results will be avoided.

It will be appreciated that one way to achieve the optimal quantity of the spike protein/fragment thereof (e.g. RBD) in the reaction mix is to prepare a solution (e.g. using a suitable buffer) containing the soluble spike protein/fragment thereof (e.g. RBD) at an appropriate concentration (e.g. by serial dilution of a stock solution), and to use an appropriate volume of the spike protein/fragment thereof (e.g. RBD)-containing solution in the reaction mix.

Another way to achieve the optimal quantity of the spike protein/fragment thereof (e.g. RBD) in the reaction mix is to provide an appropriate quantity of the spike protein/fragment thereof (e.g. RBD) to the reaction mix e.g. in a form immobilized on a solid support, e.g. in the form of the spike protein/fragment thereof (e.g. RBD) immobilized on beads.

The person of ordinary skill in the art would readily recognize permutations of the embodiments of particular interest described above may be devised, and that the articles and methods of the present disclosure can be calibrated and therefore be made quantitative or at least semi-quantitative.

In some embodiments in accordance with the various aspects of the present disclosure, the kits, compositions and methods may be characterised by reference to certain functional properties.

In some embodiments, a kit, composition and/or method according to the present disclosure may possess one or more of the following properties:
(i) Ability to detect the presence of neutralising antibodies to the SARSr-CoV in a sample with greater than 90% (e.g. one of greater than 91%, 92%, 93%, 94%, 95%, 96%, 97%, 98% or 99%, or 100%) specificity;
(ii) Ability to detect the presence of neutralising antibodies to the SARSr-CoV in a sample with greater than 90% (e.g. one of greater than 91%, 92%, 93%, 94%, 95%, 96%, 97%, 98% or 99%, or 100%) sensitivity;
(iii) Ability to detect the presence of neutralising antibodies to the SARSr-CoV in a sample without employing the relevant live SARSr-CoV;
(iv) Ability to detect the presence of neutralising antibodies to the SARSr-CoV irrespective of antibody isotype;
(v) Ability to detect the presence of neutralising antibodies to the SARSr-CoV in a sample irrespective of the species of the subject from which the sample is derived; and
(vi) Ability to arrive at a determination of the presence or absence of neutralising antibodies to the SARSr-CoV in a sample within less than 12 hours (e.g. one of less than 11 hours, 10 hours, 9 hours, 8 hours, 7 hours, 6 hours, 5 hours, 4 hours, 3 hours, 2 hours or 1 hour).

The disclosure includes the combination of the aspects and preferred features described except where such a combination is clearly impermissible or expressly avoided.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the disclosure in diverse forms thereof.

For the avoidance of any doubt, any theoretical explanations provided herein are provided for the purposes of improving the understanding of a reader. The inventor will not be bound by any of these theoretical explanations.

Where a nucleic acid sequence is disclosed herein, the reverse complement thereof is also expressly contemplated. Also, where a polypeptide-encoding nucleic acid sequence is disclosed herein equivalent polypeptide-encoding sequences as a result of degeneracy of the genetic code are also expressly contemplated.

The singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

Methods disclosed herein may be performed, or products may be present, in vitro, ex vivo, or in vivo. The term “in vitro” is intended to encompass experiments with materials, biological substances, cells and/or tissues in laboratory conditions or in culture whereas the term “in vivo” is intended to encompass experiments and procedures with intact multi-cellular organisms. In some embodiments, methods performed in vivo may be performed on non-human animals. “Ex vivo” refers to something present or taking place outside an organism, e.g. outside the human or animal body, which may be on tissue (e.g. whole organs) or cells taken from the organism.

For standard molecular biology techniques, see Sambrook, J., Russel, D. W. Molecular Cloning, A Laboratory Manual. 3 ed. 2001, Cold Spring Harbor, New York: Cold Spring Harbor Laboratory Press.

Aspects and embodiments of the present disclosure will now be discussed with reference to the accompanying figures and the following examples. Further aspects and embodiments will be apparent to those skilled in the art. All documents mentioned in this text are incorporated herein by reference in their entirety. While the disclosure has been described in conjunction with the exemplary embodiments described below, many equivalent modifications and variations will be apparent to those skilled in the art when given this disclosure. Accordingly, the exemplary embodiments of the disclosure are considered to be illustrative and not limiting. Various changes to the described embodiments may be made without departing from the spirit and scope of the disclosure.

### EXAMPLES

### Example 1: Surrogate Virus Neutralization Test

In this Example, the inventor presents a novel surrogate virus neutralization test (COVID-sVNT).

### 1.1 Materials and Methods

Recombinant His-tagged human ACE2 (hACE2, Cat #: 10108-H08H) was purchased from SinoBiologics. Recombinant SARS-CoV-2 nucleoprotein (NP) (Cat #: Z03488), spike protein 51 subunit (Cat #: Z03501) and spike protein RBD (Cat #: Z03479) were purchased from GenScript and conjugated with horse radish peroxidase (HRP) through customer service by GenScript.

1) Indirect ELISA: RBD protein (50 ng) was coated in a 96-well ELISA plate. Test serum was incubated at 1:100 dilution, after washing to remove unbound protein, followed by incubation with HRP-conjugated anti-human antibody (at a dilution of 1:10,000). After washing, HRP substrate was added for colour development.

2) Surrogate virus-host binding assay: hACE2 was coated onto wells of a 96-well plate at a concentration of 100 ng/well. After washing to remove unbound protein, HRP-conjugated recombinant nucleoprotein, S1 or RBD protein (20-100 ng) was then added to assess specific binding. After washing, HRP substrate was added for colour development.

3) Surrogate virus neutralization test (sVNT): hACE2 was coated onto wells of a 96-well plate at a concentration of 100 ng/well. In a separate plate, HRP-conjugated recombinant nucleoprotein, S1 or RBD protein (20-100 ng) was preincubated with different dilutions of test sera. The serum-HRP-protein mixes were then added to the hACE2 coated plate to assess specific inhibition/neutralization. After washing, HRP substrate was added for colour development.

### 1.2 Results

Six serum samples were used in this proof of concept study: two SARS-CoV-2 positive serum samples (COVID-55, COVID-63), two SARS-CoV positive serum samples (SARS-2, SARS-7), one negative serum sample (NEGATIVE), and one unknown serum sample (TEST-007).

The results of the above two assays are shown in the table 1 below.

TABLE 1
Results of indirect ELISA and sVNT

| Sample | ELISA¹ | sVNT-RBD² | sVNT-S1² | VNT³ |
|---|---|---|---|---|
| COVID-55 | 1.49 | +ve | +ve | +ve |
| COVID-63 | 1.62 | +ve | +ve | +ve |
| TEST-007 | 1.04 | −ve | −ve | −ve |
| SARS-2 | 0.20 | −ve | −ve | −ve |
| SARS-7 | 0.25 | −ve | −ve | −ve |
| NEGATIVE | 0.10 | −ve | −ve | −ve |

¹Average from two independent experiments
²Inhibition/neutralization at 1:40 or greater dilution
³Neutralisiton at 1:20 or greater dilution

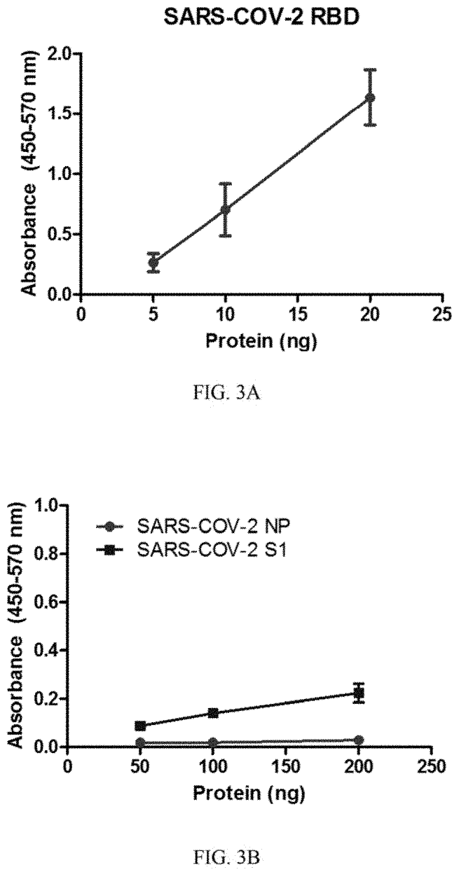

In the surrogate virus-host binding assay, when different HRP-protein conjugates were added to the hACE2 coated plate, the HRP-NP displayed no binding at all (FIG. 3B) whereas both HRP-S1 and HRP-RBD displayed significant binding (FIGS. 3A and 3B), demonstrating that the recombinant S1 and RBD proteins were functional, and suitable to be used in surrogate virus neutralization assays.

As a proof of concept, the inventor conducted the sVNT using the same panel of sera used for the indirect ELISA.

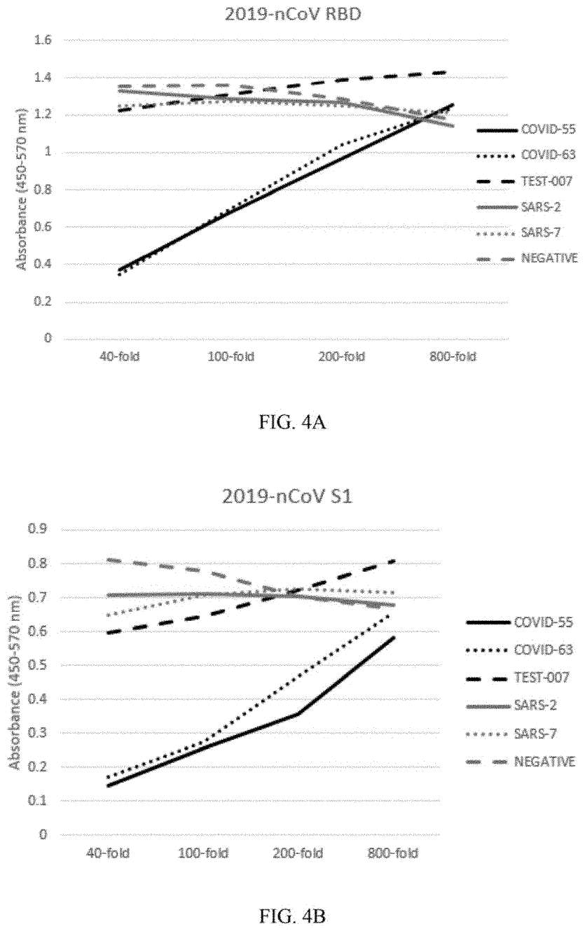

As shown in FIGS. 4A and 4B, only the two SARS-CoV-2 positive sera showed significant inhibition/neutralization in either RBD- (FIG. 4A) or S1- (FIG. 4B) based sVNT. The assay demonstrated excellent specificity, with the inhibition curves for COVID-19 samples in FIGS. 4A and 4B being completely dose and dilution dependent.

The inventor next performed analysis of 74 serum samples obtained from COVID-19 patients having been tested positive by PCR for SARS-CoV-2 infection, and 11 negative (healthy) human serum samples, using the version of the sVNT employing recombinant HRP-conjugated SARS-CoV-2 spike protein RBD.

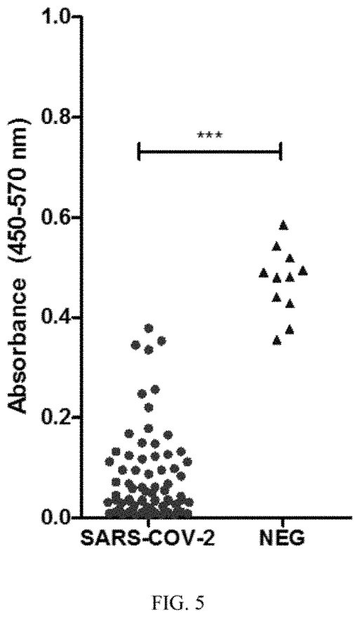

The results are shown in FIG. 5. The sVNT was able to reliably distinguish samples containing neutralising antibodies to SARS-CoV-2 from samples not containing such antibodies.

It is known that not all COVID-19 patients induce strong neutralising antibody responses to SARS-CoV-2, accounting for the results in samples determined by sVNT to be weakly-positive.

### 1.3 Conclusion

In conclusion, the inventor has developed an assay capable of detecting neutralising antibodies to SARS-CoV-2 with high sensitivity and specificity.

Importantly, the surrogate virus neutralization test is more specific than the widely-used indirect ELISA assay, as evidenced by the TEST-007 sample giving a positive reading in the indirect ELISA assay, but being shown by the surrogate virus neutralization test to be negative (as confirmed by the live virus neutralisation test).

### Example 2: SARS-CoV-2 Surrogate Virus Neutralization Test (sVNT) Based on Antibody-Mediated Blockage of ACE2-Spike (RBD) Protein Interaction

There is an urgent need for a robust serological test to detect neutralizing antibodies to SARS-CoV-2. Such a test is not only important for contact tracing, but for determining infection rate, herd immunity and predicted humoral protection. The current gold standard is a virus neutralization test (VNT) requiring live virus and a biosafety level 3 (BSL3) laboratory. On the other hand, the ELISA- or lateral flow-based assays are for the detection of binding antibodies, which does not directly correlate with their neutralizing ability. Here the inventor describes a SARS-CoV-2 surrogate virus neutralization test (sVNT) that is designed to detect total neutralizing antibodies in an isotype- and species-independent manner. This simple and rapid test is based on antibody-mediated blockage of virus-host interaction between the ACE2 receptor protein and the receptor binding domain (RBD) of the viral spike protein. The test has been validated with two COVID-19 patient cohorts in two different countries, achieving 100% specificity and 95-100% sensitivity, and is capable of differentiating antibody responses from other known human coronaviruses. Importantly, the sVNT does not require BSL3 containment, thereby making the test immediately accessible to the global community.

### 2.1 Introduction

The COVID-19 outbreak was first recognized in December 2019 in Wuhan, China¹, which has since spread to all parts of the world resulting in a total 2,160,207 infections with 146,088 deaths as of 18 Apr. 2020². The causative agent was identified as 2019-nCoV, subsequently designated SARS-CoV-2^{3,4}, which belongs to the species SARS-related coronavirus (SARSr-CoV), same as for SARS-CoV, the causative agent of the SARS outbreak 17 years ago⁵.

While molecular detection, such as polymerase chain reaction (PCR) and next generation sequencing (NGS), played and continue to play an important role in acute diagnosis and monitoring of genetic changes of the virus, there is now an urgent need for a reliable and versatile serological or antibody test. Such a test is needed for retrospective contact tracing, investigation of asymptomatic infection rate, accurate determination of case fatality rate, assessment of herd immunity and humoral protective immunity in recovered patients and recipients of vaccine candidates, and in the search for the natural reservoir host and intermediate host(s) [6]. Research laboratories and pharmaceutical companies are racing to produce antibody tests that can detect SARS-CoV-2 infection with sufficient specificity and sensitivity [6]. There are two types of antibody tests one can aim for. The first type is the virus neutralization test (VNT) which detects neutralizing antibodies (NAbs) in a patient's blood. VNT requires handling live SARS-CoV-2 in a specialized biosafety level 3 (BSL3) containment facility which is tedious and time consuming, taking 2-4 days to complete. Pseudovirus-based virus neutralization test (pVNT) is similar, but still requires the use of live viruses and cells although handled in a BSL2 laboratory [7, 8]. All other assays, such as ELISA and lateral flow rapid tests, represent the second assay type which detect only binding antibodies, and not NAbs [6, 9-11].

In this Example the inventor describes a surrogate virus neutralization test (sVNT) which detects NAbs, but without the need to use any live virus or cells and can be completed in 1-2 hours in a BSL2 lab. Using purified receptor binding domain (RBD) protein from the viral spike (S) protein and the host cell receptor ACE2, the test is designed to mimic the virus-host interaction by direct protein-protein interaction in a test tube or an ELISA plate well. This highly specific interaction can then be neutralized, i.e., blocked by highly specific NAbs in patient or animal sera in the same manner as in a conventional VNT.

### 2.2 Materials and Methods

Human embryonic kidney (HEK293T) cells (ATCC #CRL-3216) and African green monkey kidney, clone E6 (Vero-E6) cells (ATCC #CRL-1586) were maintained in Dulbecco's modified Eagle Medium (DMEM) supplemented with 10% fetal bovine serum. SARS-CoV-2, isolate BetaCoV/Singapore/2/2020 (Accession ID EPI_ISL_406973), was used for virus neutralization test on Vero-E6 cells as described previously [26].

In Singapore, COVID-19 patient sera used in this study was from the Singapore PROTECT study as described [13]. Sera from recovered SARS patients from 2003 were as previously described [15]. For SARS recall sampling in 2020, the inventor obtained blood from consenting individuals previously admitted for SARS (ethics approval number: NHG DSRB E 2020/00091). The hCoV serum panel included post-infection samples from subjects confirmed CoV 229/NL63 and CoV OC43 positive using the SeeGene RV12 respiratory multiplex kit in a previous study (ethics approval number: NUS-IRB 11-3640) [27]. Negative control sera were obtained from residual serum samples from previous unrelated studies.

In Nanjing, China, COVID-19 convalescent sera were collected with written informed consent and approved by the ethics committee of the Second Hospital of Nanjing (ethics approval number: 2020-LS-ky003). Rabbit anti-SARS-CoV-2 RBD sera were purchased from GenScript. Rabbit and ferret anti-SARS-CoV sera, and alpaca anti-MERS-CoV sera were as described in previous studies [28, 29].

Direct Binding and sVNT Assay

For direct binding, hACE2 protein (GenScript, Cat #: Z03484) was coated at 100 ng/well in 100 mM carbonate-bicarbonate coating buffer (pH 9.6). HRP-conjugated SARS-CoV-2 nucleocapsid (briefly referred to as N hereinafter, GenScript, Cat #: Z03488), 51 (GenScript, Cat #: Z03501), RBD (GenScript, Cat #: Z03479) or HRP-conjugated SARS-CoV-RBD (customer-made by GenScript) was added to the hACE2 coated plate at different concentration in OptEIA assay diluent (BD) for 1 h at room temperature. Unbound HRP-conjugated antigens were removed by five phosphate buffered saline, 0.05% tween-20 (PBST) washes. Colorimetric signal was developed on the enzymatic reaction of HRP with chromogenic substrate, 3,3′,5,5′-tetramethylbenzidine (TMB) (Invitrogen). Equal volume of TMB stop solution (KPL) was added to stop the reaction, and the absorbance reading at 450 nm and 570 nm were acquired using Cytation 5 microplate reader (BioTek). For the surrogate neutralization test (sVNT), 6 ng of HRP-RBD (from SARS-CoV or SARS-CoV-2) was pre-incubated with test serum at the final dilution of 1:20 for 1 h at 37° C., followed by hACE2 incubation for 1 h at room temperature. Inhibition (%)=(1−Sample OD value/Negative Control OD value)×100.

### Indirect ELISA

SARS-CoV-2 N protein and SARS-CoV N protein were expressed from the pcDNA3.1 SARS-CoV-2 N and pDualGC SARS-CoV N transfected HEK293T cells and purified using Ni Sepharose (GE healthcare). For indirect ELISA, 100 ng of each protein was coated onto MaxiSORP ELISA plate (Nunc) using 100 mM carbonate buffer and blocked with BD OptEIA (BD). COVID-19 and SARS patient sera were tested at a dilution of 1:50 and detected by Goat-anti-human IgG-HRP (Santa Cruz) at 1:10,000 dilution. The chromogenic signal was developed using TMB substrate (Invitrogen) and the reaction was stop with TMB stop solution (KPL). Absorbance readings at 450 and 570 nm were obtained using Cytation 5 microplate reader (Bio-Tek).

### Capture ELISA

96-well Maxisorp plates (Nunc) were coated with 10 μg/ml of anti-human IgM (SeraCare) or anti-human IgG (Jackson labs) in bicarbonate buffer overnight at 4° C. Wells were blocked using BD OptEIA assay diluent (BD) for 1 h at 37° C., and heat-inactivated sera diluted 1:50 were next added and incubated for 1 h at 37° C. Following extensive washing, SARS-CoV-2-RBD-HRP (GenScript) diluted 4 μg/ml was added and incubated for 30 min at 37° C. Chromogenic reaction was quantified following the addition of TMB substrate (Invitrogen) and stop solution (KPL SeraCare). The absorbance of the samples was measured at 450 nm and the background at 570 nm. Negative controls consisted of 37 naïve human sera. Results are presented as fold-change over average reading of negative controls.

### Statistical Analysis

Statistical analysis was perform using GraphPad Prism software with the Kruskal-Wallis test to compare multiple groups, followed by Dunn's multiple comparisons test. Data were considered significant if * P<0.05, **P<0.01, ***P<0.001, ****P<0.0001.

### 2.3 Results

### Biochemical Simulation of Virus-Receptor Interaction and Antibody-Mediated Neutralization

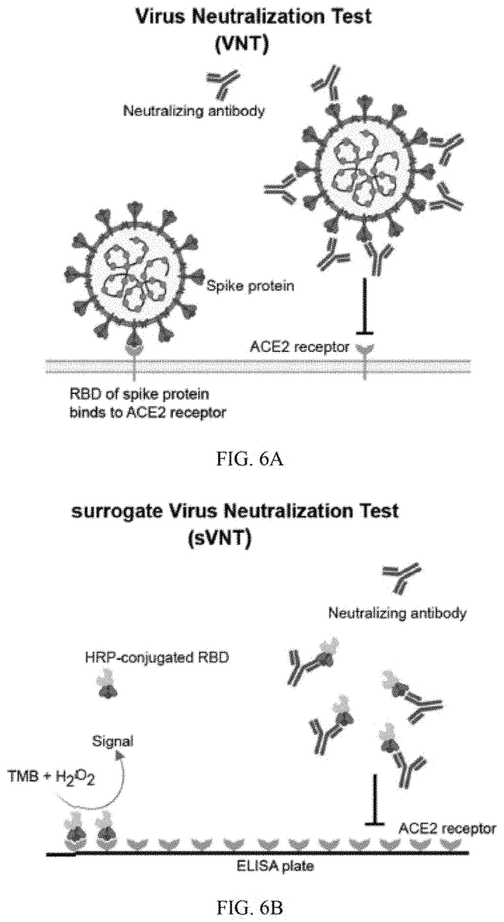

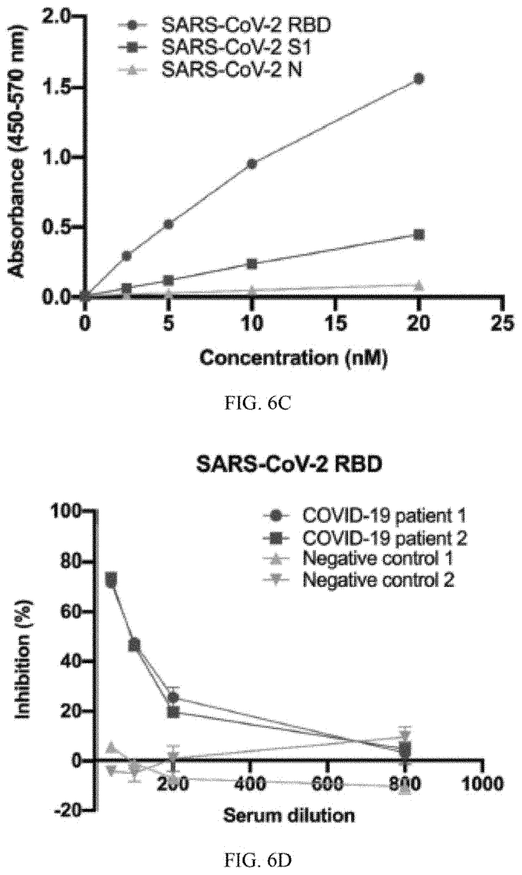

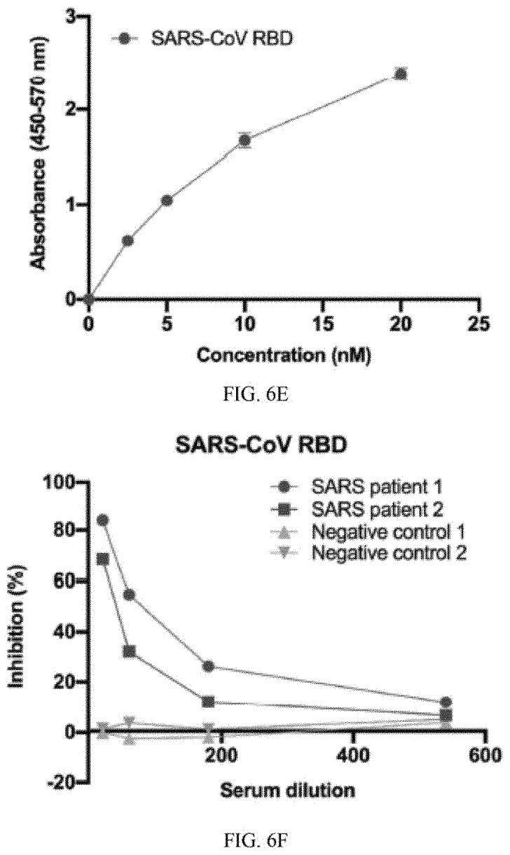

Immediately after SARS-CoV-2 was identified as the causative agent of the COVID-19 outbreak, it was shown that the human angiotensin converting enzyme-2 (hACE2) is the main functional receptor for viral entry [3]. The inventor hypothesized that the virus-receptor binding can be mimicked in vitro via a protein-protein interaction using purified recombinant hACE2 and the RBD of the SARS-CoV-2 S protein. This interaction can be blocked by virus NAbs present in the test serum, using the same principle as a conventional VNT conducted using live virus inside a BSL3 facility (FIGS. 6A and 6B).

Direct binding was demonstrated using different SARS-CoV-2 proteins conjugated with horseradish peroxidase (HRP). There is a dose-dependent specific binding between hACE2 and RBD or S1, but not with the nucleocapsid (N) protein, with the RBD producing the best binding characteristics (FIG. 6C). The HRP-RBD protein was chosen for subsequent studies. It was then demonstrated that the specific RBD-hACE2 binding can be blocked or neutralized by COVID-19 sera in a dose-dependent manner, but not by sera from healthy controls (FIG. 6D). To prove that the same principle works with the closely related SARS-CoV, which also uses hACE2 as the entry receptor [12], similar experiments were repeated and proved that the SARS-CoV RBD performed in an almost identical manner in this new test format (FIGS. 6E and 6F), termed surrogate virus neutralization test (sVNT).

### Isotype- and Species-Independent Neutralization

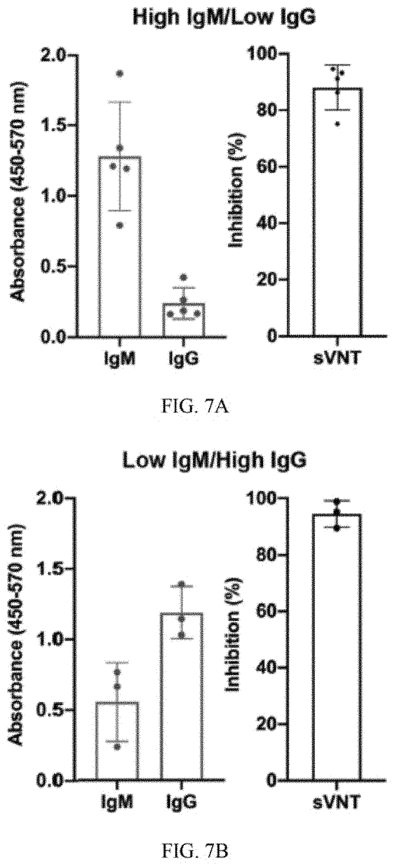

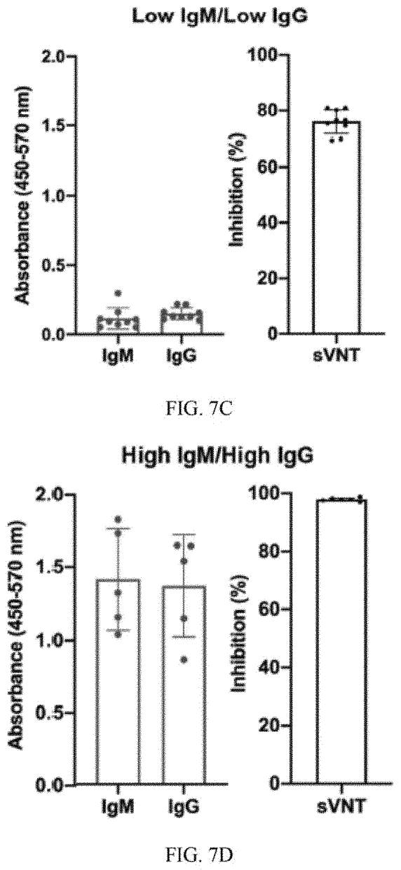

One of the advantages of the sVNT is its ability to detect total antibodies in patient sera, in contrast to most SARS-CoV-2 antibody tests published or marketed, which are almost all isotype-specific, mostly for IgM or IgG, with some for IgA [9-11]. From a panel of 77 COVID-19 positive sera from patients in Singapore, the inventor has designated four groups based on IgM or IgG ELISA levels, determined by in-house capture ELISA assays (see Methods), present in the patient convalescent sera: a) high IgM/low IgG; b) low IgM/high IgG; c) low IgM/low IgG; and d) high IgM/high IgG. All groups showed strong neutralization activity in the sVNT (FIGS. 7A-7D), demonstrating the isotype-independent performance of the assay. It is worth to note that for panel c with low IgM/IgG, the % inhibition in sVNT is still significant at 70-75%, demonstrating its superior sensitivity as this group of sera were deemed negative or weakly positive with isotype-specific capture ELISA based on IgM or IgG alone.

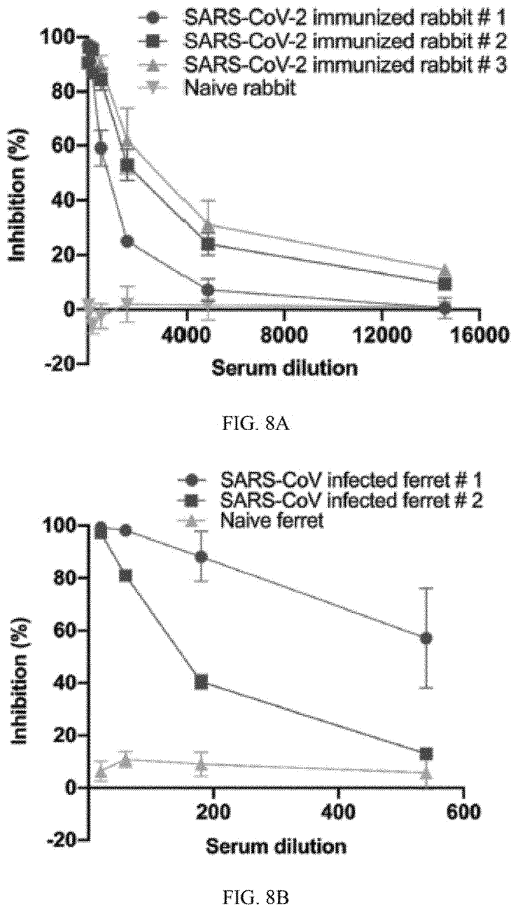

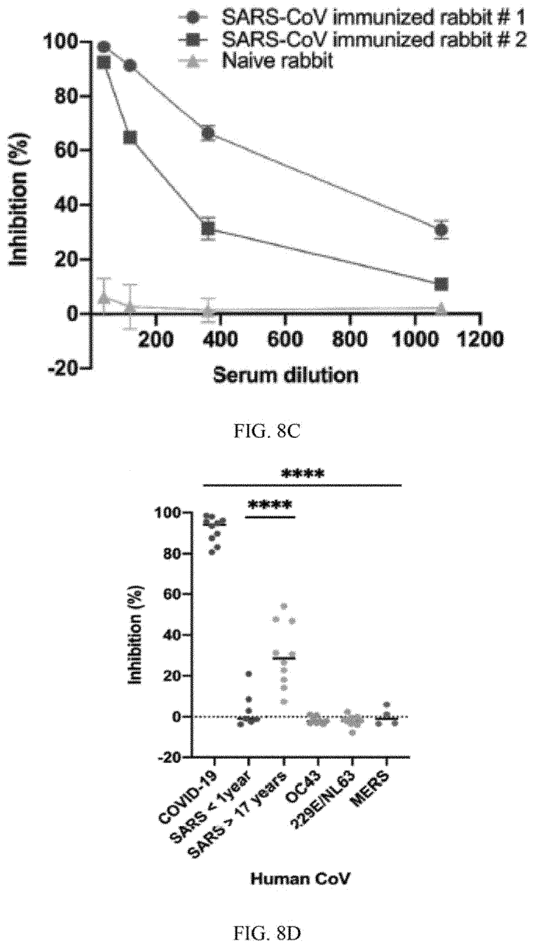

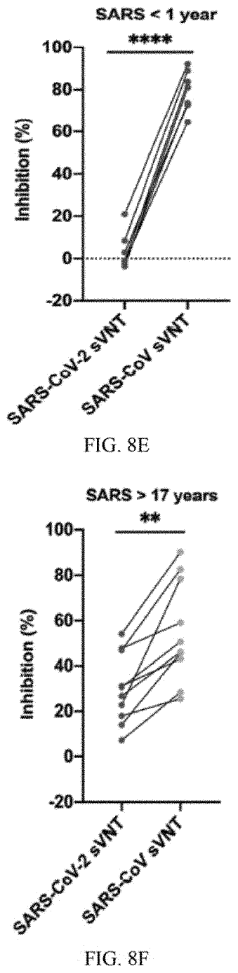

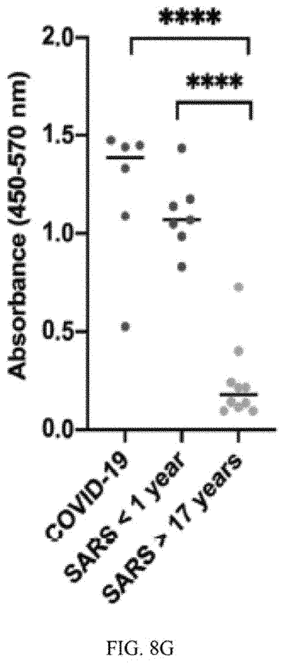

The inventor then tested different animal sera in the sVNT assays to demonstrate species-independent performance. Results from three independent rabbits immunized with the SARS-CoV-2 RBD protein, demonstrate very potent neutralizing activity in the SARS-CoV-2 sVNT (FIG. 8A). Similarly, sera from ferrets infected with SARS-CoV (FIG. 8B) and rabbits immunized with inactivated SARS-CoV (FIG. 8C) also showed an efficient dose-dependent inhibition of the hACE2-SARS-CoV RBD interaction in the SARS-CoV sVNT.

Specificity Against Other hCoVs and Comparison of SARS Sera Collected in 2003 vs 2020

To demonstrate specificity, the inventor tested different panels of sera against other known human coronaviruses (hCoVs) and confirmed that the SARS-CoV-2 sVNT can differentiate antibody responses between COVID-19 and other coronavirus infections (FIG. 8D). For SARS sera, there is some level of cross reactivity as expected from their close genetical relatedness and previous published studies [3, 7]. But the difference in neutralization is statistically significant, and hence the sVNT can be used to differentiate SARS-CoV-2 infection from past SARS infection. For human sera from patients with 229/NL63 or OC43 infection and alpaca sera from experimental MERS-CoV infection, there is no detectable cross neutralization.

During the investigation of potential cross reactivity between SARS sera and SARS-CoV-2 virus, several important observations were made. Firstly, despite the lack of cross neutralization by SARS sera against the live SARS-CoV-2 virus in VNT observed by us and other groups [13], some level of cross neutralization in sVNT was detected (FIG. 8D), indicating sVNT is more sensitive than VNT. Secondly, SARS NAbs are detectable for at least 17 years in recovered patients (FIG. 8F). Thirdly, the cross neutralization level is higher in the 2020 SARS sera than the 2003 samples (FIG. 8D) although the homologous neutralizing level of the 2020 SARS sera (FIG. 8F) is lower than the 2003 SARS sera (FIG. 8E). Lastly, the N-specific antibody level was found to be much lower in the 2020 SARS sera than the 2003 samples (FIG. 8G).

Correlation Between Live Virus VNT and Biochemical sVNT

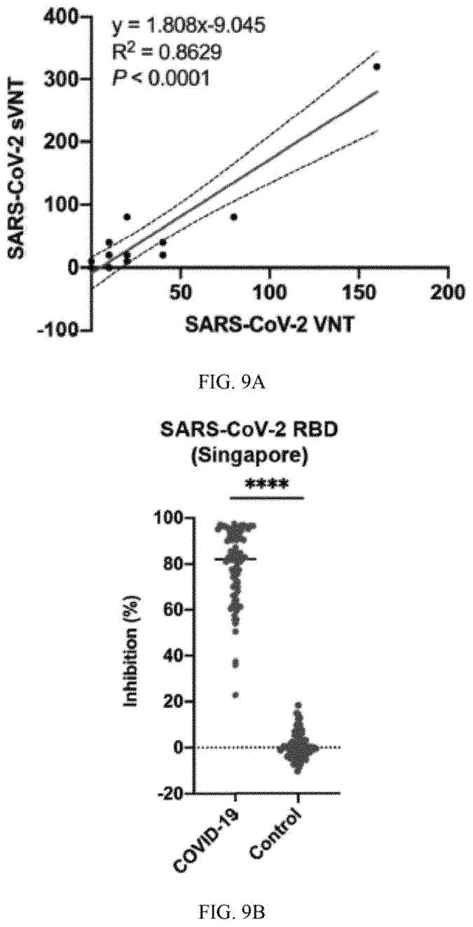

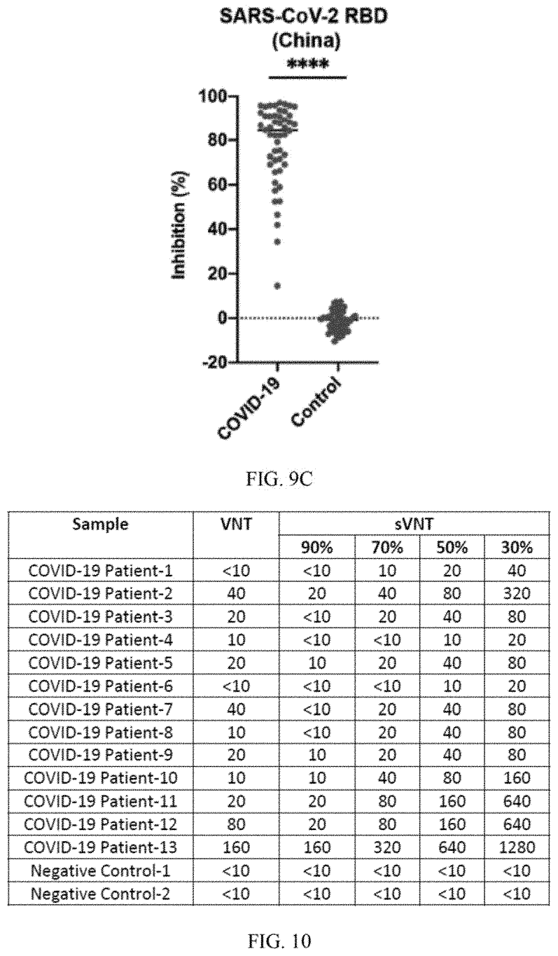

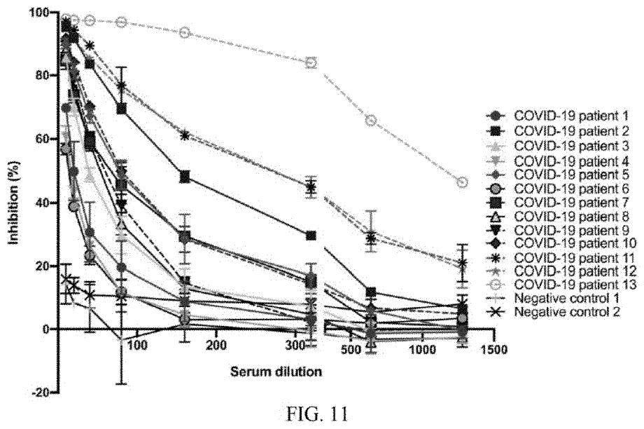

A panel of COVID-19 sera with different levels of SARS-CoV-2 NAbs as shown by sVNT (FIG. 11) were chosen for a comparative and correlation study between the live virus based VNT and the RBD-hACE2 based sVNT. The results demonstrate good overall correlation between the two assays (FIG. 9A and FIG. 10). The SARS-CoV-2 sVNT is more sensitive than VNT. At the 50% inhibition cutoff, which is considered a stringent cutoff as evident from the titration curves in FIG. 11, all COVID-19 patient sera showed neutralization at 1:20 or greater with the COVID-19 Patient 13 serum reaching a neutralization titer equal to or greater than 640 (FIG. 11).

Validation with Two Cohorts of Positive and Negative Sera from Two Countries

To validate the performance of the SARS-CoV-2 sVNT, two different cohorts of positive and negative sera were analysed. The assay was performed in two different countries by two independent groups to further assure reliability and reproducibility. For the first cohort, 77 sera from PCR-confirmed COVID-19 patients in Singapore collected on days 14-33 after symptom onset and 75 healthy control sera were tested. All control sera were negative, resulting in a 100% specificity. Using a cutoff at 20% inhibition, the assay sensitivity is at 100%. The sensitivity decreases to 95.6% when a 40% cutoff is used (FIG. 9B). For the second cohort, 50 sera each of healthy controls and PCR-confirmed COVID-19 patients in Nanjing, China, sampled on days 27-61 after symptom onset were tested. The specificity is 100%. The sensitivity is 98% and 96% using a 20% and 40% cutoff, respectively (FIG. 9C).

### 2.3 Discussion

We are more than 100 days into the COVID-19 outbreak and attention worldwide, both in the scientific community and for policy makers, has shifted focus from acute diagnostic strategy and capacity to the use of serology for the “exit strategy”, relying on accurate assessment of infection prevalence at the individual and population (herd) level. Discussion and debate on the role of serology has intensified greatly in this context [6].

While there are many COVID-19 lab-based or point-of-care (POC) antibody test kits commercially available, none are capable of measuring NAbs. VNT or pVNT remain the only platform for detection of NAbs. Both require live virus and cells, highly skilled operators, are less sensitive in general, and take days to obtain results. VNT and pVNT are thus not suitable for mass production and testing, even in the most developed nations.

The World Health Organization (WHO) has recently cautioned that positive results from antibody tests do not equal to protective immunity [14] due to two aspects or obstacles. Firstly, most, if not all, current testing done at large scale is for detection of binding antibodies only and does not measure NAbs; secondly, the presence of NAbs may or may not correlate with protection. While the second aspect will take much more in-depth scientific and clinical research to resolve in the specific context of COVID-19 infection, past experiences with viral infection in general argue that in most recovered patients NAb level is a good indicator of protective immunity, despite the fact that some patients may not obey this “rule of thumb” [15, 16]. In this Example, the inventor describes a novel sVNT platform to tackle the first obstacle.

The data presented here demonstrated that sVNT is as specific as, and more sensitive than VNT. The results obtained from sVNT correlates well with VNT. The major advantage of sVNT is that it can be rapidly conducted in most research or clinical labs without the need to use live biological materials and biosafety containment. The sVNT is also amenable to high throughput testing and/or fully automated testing after minimal adaptation.

Another advantage of sVNT is its ability to detect SARS-CoV-2 antibodies in a species-independent manner. As the origin of SARS-CoV-2 and early transmission event remain elusive, the sVNT assay will be ideally suited for “virus hunting” as past studies have amply demonstrated that serological surveys are more superior than molecular detection as the virus-specific antibodies last much longer in animals than the viral genetic material [17-19]. Sampling serum for antibody detection is also more reliable than other sampling approaches used for molecular detection as the target tissues can vary from virus to virus [20-22].

In addition, sVNT offers a key advantage over most ELISA or POC tests in its ability to detect total NAbs in an isotype-independent manner. This will not only simplify the testing strategy, but also further increase test sensitivity. As shown in FIG. 7C for the serum panel of COVID-19 patients showing low IgM and IgG in the isotype-specific ELISAs, the sVNT assay still detected significant level of NAbs. Although the mechanism needs further investigation, there are at least two possibilities: the presence of other Ig isotypes or neutralization synergy or cooperativity from the combination of different isotype antibodies targeting different neutralization critical epitopes, as previously observed for HIV and other viruses [23-25].

Results obtained for the two SARS serum panels are very interesting. The long lasting NAbs 17 years after initial infection is encouraging news for recovered COVID-19 patients considering the close relationship of the two viruses. The mechanism and biological significance of the increased cross neutralization towards SARS-COV-2 coupled with the decrease/disappearance of N-specific antibodies 17 years after infection warrants further investigation in the context of better understanding SARSr-CoV immune response dynamics.

In summary, the inventor has addressed the challenge of COVID-19 serology with a new approach that enables the detection of NAbs in an easy, safe, rapid and inexpensive manner with enhanced specificity and sensitivity. The data indicate that their performance is generally well correlated. Its application can cover many aspects of COVID-19 investigation from contact tracing, sero-prevalence survey, reservoir/intermediate animal tracking to assessment of herd immunity, longevity of protective immunity and efficacy of different vaccine candidates.

Exemplary sequences of the disclosure are set forth below.

| SEQ ID | | |
|---|---|---|
| NO: | DESCRIPTION | SEQUENCE |
| 1 | SARS-CoV spike | MFIFLLFLTLTSGSDLDRCTTFDDVQAPNYTQHTSSMRGVYYPDEIFRSDTLYLTQDLFLPFY |
| | protein | SNVTGFHTINHTFDNPVIPFKDGIYFAATEKSNVVRGWVFGSTMNNKSQSVIIINNSTNVVIR |
| | | ACNFELCDNPFFAVSKPMGTQTHTMIFDNAFNCTFEYISDAFSLDVSEKSGNFKHLREFVFK |
| | | NKDGFLYVYKGYQPIDVVRDLPSGFNTLKPIFKLPLGINITNFRAILTAFSPAQDTWGTSAAA |
| | | YFVGYLKPTTFMLKYDENGTITDAVDCSQNPLAELKCSVKSPEIDKGIYQTSNFRVVPSGDV |
| | | VRFPNITNLCPFGEVFNATKFPSVYAWERKKISNCVADYSVLYNSTFFSTFKCYGVSATKLN |
| | | DLCFSNVYADSFVVKGDDVRQIAPGQTGVIADYNYKLPDDFMGCVLAWNTRNEDATSTGN |
| | | YNYKYRYLRHGKLRPFERDISNVPFSPDGKPCTPPALNCYWPLNDYGFYTTTGIGYQPYRV |
| | | VVLSFELLNAPATVCGPKLSTDLIKNQCVNFNFNGLTGTGVLTPSSKRFQPFQQFGRDVSDF |
| | | TDSVRDPKTSEILDISPCSFGGVSVITPGTNASSEVAVLYQDVNCTDVSTAIHADQLTPAWRI |
| | | YSTGNNVFQTQAGCLIGAEHVDTSYECDIPIGAGICASYHTVSLLRSTSQKSIVAYTMSLGAD |
| | | SSIAYSNNTIAIPTNFSISITTEVMPVSMAKTSVDCNMYICGDSTECANLLLQYGSFCTQLNRA |
| | | LSGIAAEQDRNTREVFAQVKQMYKTPTLKYFGGFNFSQILPDPLKPTKRSFIEDLLFNKVTLA |
| | | DAGFMKQYGECLGDINARDLICAQKFNGLTVLPPLLTDDMIAAYTAALVSGTATAGWTFG |
| | | AGAALQIPFAMQMAYRFNGIGVTQNVLYENQKQIANQFNKAISQIQESLTTTSTALGKLQD |
| | | VVNQNAQALNTLVKQLSSNFGAISSVLNDILSRLDKVEAEVQIDRLITGRLQSLQTYVTQQLI |
| | | RAAEIRASANLAATKMSECVLGQSKRVDFCGKGYHLMSFPQAAPHGVVFLHVTYVPSQER |
| | | NFTTAPAICHEGKAYFPREGVFVFNGTSWFITQRNFFSPQIITTDNTFVSGNCDVVIGIINNTV |
| | | YDPLQPELDSFKEELDKYFKNHTSPDVDLGDISGINASVVNIQKEIDRLNEVAKNLNESLIDL |
| | | QELGKYEQYIKWPWYVWLGFIAGLIAIVMVTILLCCMTSCCSCLKGACSCGSCCKFDEDDS |
| | | EPVLKGVKLHYT |
| 2 | SARS-CoV | MYSFVSEETGTLIVNSVLLFLAFVVFLLVTLAILTALRLCAYCCNIVNVSLVKPTVYVYSRV |
| | envelope protein | KNLNSSEGVPDLLV |
| 3 | SARS-CoV | MADNGTITVEELKQLLEQWNLVIGFLFLAWIMLLQFAYSNRNRFLYIIKLVFLWLLWPVTL |
| | membrane protein | ACFVLAAVYRINWVTGGIAIAMACIVGLMWLSYFVASFRLFARTRSMWSFNPETNILLNVP |
| | | LRGTIVTRPLMESELVIGAVIIRGHLRMAGHSLGRCDIKDLPKEITVATSRTLSYYKLGASQR |
| | | VGTDSGFAAYNRYRIGNYKLNTDHAGSNDNIALLVQ |
| 4 | SARS-CoV | MSDNGPQSNQRSAPRITFGGPTDSTDNNQNGGRNGARPKQRRPQGLPNNTASWFTALTQH |
| | nucleocapsid | GKEELRFPRGQGVPINTNSGPDDQIGYYRRATRRVRGGDGKMKELSPRWYFYYLGTGPEAS |
| | protein | LPYGANKEGIVWVATEGALNTPKDHIGTRNPNNNAATVLQLPQGTTLPKGFYAEGSRGGSQ |
| | | ASSRSSSRSRGNSRNSTPGSSRGNSPARMASGGGETALALLLLDRLNQLESKVSGKGQQQQ |
| | | GQTVTKKSAAEASKKPRQKRTATKQYNVTQAFGRRGPEQTQGNFGDQDLIRQGTDYKHW |
| | | PQIAQFAPSASAFFGMSRIGMEVTPSGTWLTYHGAIKLDDKDPQFKDNVILLNKHIDAYKTF |
| | | PPTEPKKDKKKKTDEAQPLPQRQKKQPTVTLLPAADMDDFSRQLQNSMSGASADSTQA |
| 5 | SARS-CoV-2 | MFLLTTKRTMFVFLVLLPLVSSQCVNLTTRTQLPPAYTNSFTRGVYYPDKVFRSSVLHSTQD |
| | spike protein | LFLPFFSNVTWFHAIHVSGTNGTKRFDNPVLPFNDGVYFASTEKSNIIRGWIFGTTLDSKTQS |
| | | LLIVNNATNVVEKVCEFQFCNDPFLGVYYHKNNKSWMESEFRVYSSANNCTFEYVSQPFLM |
| | | DLEGKQGNFKNLREFVFKNIDGYFKIYSKHTPINLVRDLPQGFSALEPLVDLPIGINITRFQTL |
| | | LALHRSYLTPGDSSSGWTAGAAAYYVGYLQPRTFLLKYNENGTITDAVDCALDPLSETKCT |
| | | LKSFTVEKGIYQTSNFRVQPTESIVRFPNITNLCPFGEVFNATRFASVYAWNRKRISNCVADY |
| | | SVLYNSASFSTFKCYGVSPTKLNDLCFTNVYADSFVIRGDEVRQIAPGQTGKIADYNYKLPD |
| | | DFTGCVIAWNSNNLDSKVGGNYNYLYRLFRKSNLKPFERDISTEIYQAGSTPCNGVEGFNC |
| | | YFPLQSYGFQPTNGVGYQPYRVVVLSFELLHAPATVCGPKKSTNLVKNKCVNFNFNGLTGT |
| | | GVLTESNKKFLPFQQFGRDIADTTDAVRDPQTLEILDITPCSFGGVSVITPGTNTSNQVAVLY |
| | | QDVNCTEVPVAIHADQLTPTWRVYSTGSNVFQTRAGCLIGAEHVNNSYECDIPIGAGICASY |
| | | QTQTNSPRRARSVASQSIIAYTMSLGAENSVAYSNNSIAIPTNFTISVTTEILPVSMTKTSVDC |
| | | TMYICGDSTECSNLLLQYGSFCTQLNRALTGIAVEQDKNTQEVFAQVKQIYKTPPEKDFGGF |
| | | NFSQILPDPSKPSKRSFIEDLLFNKVTLADAGFEKQYGDCLGDIAARDLICAQKFNGLTVLPPL |
| | | LTDEMIAQYTSALLAGTITSGWTFGAGAALQIPFAMQMAYRFNGIGVTQNVLYENQKLIAN |
| | | QFNSAIGKIQDSLSSTASALGKLQDVVNQNAQALNTLVKQLSSNFGAISSVLNDILSRLDKV |
| | | EAEVQIDRLITGRLQSLQTYVTQQLIRAAEIRASANLAATKMSECVLGQSKRVDFCGKGYHL |
| | | MSFPQSAPHGVVFLHVTYVPAQEKNFTTAPAICHDGKAHFPREGVFVSNGTHWFVTQRNFY |
| | | EPQIITTDNTFVSGNCDVVIGIVNNTVYDPLQPELDSFKEELDKYFKNHTSPDVDLGDISGIN |
| | | ASVVNIQKEEDRLNEVAKNLNESLIDLQELGKYEQYEKWPWYIWLGFIAGLIAIVMVTEVILC |
| | | CMTSCCSCLKGCCSCGSCCKFDEDDSEPVLKGVKLHYT |
| 6 | SARS-CoV-2 | MYSFVSEETGTLIVNSVLLFLAFVVFLLVTLAILTALRLCAYCCNIVNVSLVKPSFYVYSRVK |
| | envelope protein | NLNSSRVPDLLV |
| 7 | SARS-CoV-2 | MADSNGTITVEELKKLLEQWNLVIGFLFLTWICLLQFAYANRNRFLYIEKLIFLWLLWPVTL |
| | membrane protein | ACFVLAAVYRINVVITGGIAIAMACLVGLMWLSYFIASFRLFARTRSMWSFNPETNILLNVPL |
| | | HGTILTRPLLESELVIGAVILRGHLRIAGHHLGRCDEKDLPKEITVATSRTLSYYKLGASQRVA |
| | | GDSGFAAYSRYRIGNYKLNTDHSSSSDNIALLVQ |
| 8 | SARS-CoV-2 | MSDNGPQNQRNAPRITFGGPSDSTGSNQNGERSGARSKQRRPQGLPNNTASWFTALTQHGK |
| | nucleocapsid | EDLKFPRGQGVPINTNSSPDDQIGYYRRATRRIRGGDGKMKDLSPRWYFYYLGTGPEAGLP |
| | protein | YGANKDGIIWVATEGALNTPKDHIGTRNPANNAAIVLQLPQGTTLPKGFYAEGSRGGSQAS |
| | | SRSSSRSRNSSRNSTPGSSRGTSPARMAGNGGDAALALLLLDRLNQLESKMSGKGQQQQGQ |
| | | TVTKKSAAEASKKPRQKRTATKAYNVTQAFGRRGPEQTQGNFGDQELIRQGTDYKHWPQI |
| | | AQFAPSASAFFGMSRIGMEVTPSGTVVLTYTGAEKLDDKDPNFKDQVILLNKHIDAYKTFPPT |
| | | EPKKDKKKKADETQALPQRQKKQQTVTLLPAADLDDFSKQLQQSMSSADSTQA |
| 9 | SARS-CoV spike | SDLDRCTTFDDVQAPNYTQHTSSMRGVYYPDEIFRSDTLYLTQDLFLPFYSNVTGFHTINHT |
| | protein S1 subunit | FDNPVIPFKDGIYFAATEKSNVVRGWVFGSTMNNKSQSVIIINNSTNVVIRACNFELCDNPFF |
| | | AVSKPMGTQTHTMIFDNAFNCTFEYISDAFSLDVSEKSGNFKHLREFVFKNKDGFLYVYKG |
| | | YQPIDVVRDLPSGFNTLKPIFKLPLGINITNFRAILTAFSPAQDTWGTSAAAYFVGYLKPTTF |
| | | MLKYDENGTITDAVDCSQNPLAELKCSVKSFEEDKGIYQTSNFRVVPSGDVVRFPNITNLCPF |
| | | GEVFNATKFPSVYAWERKKISNCVADYSVLYNSTFFSTFKCYGVSATKLNDLCFSNVYADS |
| | | FVVKGDDVRQIAPGQTGVIADYNYKLPDDFMGCVLAWNTRNIDATSTGNYNYKYRYLRH |
| | | GKLRPFERDISNVPFSPDGKPCTPPALNCYWPLNDYGFYTTTGIGYQPYRVVVLSIELLNAP |
| | | ATVCGPKLSTDLEKNQCVNFNFNGLTGTGVLTPSSKRFQPFQQFGRDVSDFTDSVRDPKTSEI |
| | | LDISPCSFGGVSVITPGTNASSEVAVLYQDVNCTDVSTAIHADQLTPAWRIYSTGNNVFQTQ |
| | | AGCLIGAEHVDTSYECDIPIGAGICASYHTVSLLR |
| 10 | SARS-CoV spike | RVVPSGDVVRFPNITNLCPFGEVFNATKFPSVYAWERKKISNCVADYSVLYNSTFFSTFKCY |
| | protein RBD | GVSATKLNDLCFSNVYADSFVVKGDDVRQIAPGQTGVIADYNYKLPDDFMGCVLAWNTR |
| | | NIDATSTGNYNYKYRYLRHGKLRPFERDISNVPFSPDGKPCTPPALNCYWPLNDYGFYTTTG |
| | | IGYQPYRVVVLSFELLNAPATVCGPKLSTDLIKNQCVNF |
| 11 | SARS-CoV spike | NTRNIDATSTGNYNYKYRYLRHGKLRPFERDISNVPFSPDGKPCTPPALNCYWPLNDYGFY |
| | protein RBM | TTTGIGYQPY |
| 12 | SARS-CoV-2 | SQCVNLTTRTQLPPAYTNSFTRGVYYPDKVFRSSVLHSTQDLFLPFFSNVTVVFHAIHVSGTN |
| | spike protein S1 | GTKRFDNPVLPFNDGVYFASTEKSNIIRGWIFGTTLDSKTQSLLIVNNATNVVIKVCEFQFCN |
| | subunit | DPFLGVYYHKNNKSWMESEFRVYSSANNCTFEYVSQPFLMDLEGKQGNFKNLREFVFKNI |
| | | DGYFKIYSKHTPINLVRDLPQGFSALEPLVDLPIGINITRFQTLLALHRSYLTPGDSSSGWTAG |
| | | AAAYYVGYLQPRTFLLKYNENGTITDAVDCALDPLSETKCTLKSFTVEKGIYQTSNFRVQPT |
| | | ESIVRFPNITNLCPFGEVFNATRFASVYAWNRKRISNCVADYSVLYNSASFSTFKCYGVSPTK |
| | | LNDLCFTNVYADSFVIRGDEVRQIAPGQTGKIADYNYKLPDDFTGCVIAWNSNNLDSKVGG |
| | | NYNYLYRLFRKSNLKPFERDISTEIYQAGSTPCNGVEGFNCYFPLQSYGFQPTNGVGYQPYR |
| | | VVVLSFELLHAPATVCGPKKSTNLVKNKCVNFNFNGLTGTGVLTESNKKFLPFQQFGRDIA |
| | | DTTDAVRDPQTLEILDITPCSFGGVSVITPGTNTSNQVAVLYQDVNCTEVPVAIHADQLTPT |
| | | WRVYSTGSNVFQTRAGCLIGAEHVNNSYECDIPIGAGICASYQTQTNSPRRAR |
| 13 | SARS-CoV-2 | RVQPTESIVRFPNITNLCPFGEVFNATRFASVYAWNRKRISNCVADYSVLYNSASFSTFKCY |
| | spike protein | GVSPTKLNDLCFTNVYADSFVIRGDEVRQIAPGQTGKIADYNYKLPDDFTGCVIAWNSNNL |
| | RBD | DSKVGGNYNYLYRLFRKSNLKPFERDISTEIYQAGSTPCNGVEGFNCYFPLQSYGFQPTNGV |
| | | GYQPYRVVVLSFELLHAPATVCGPKKSTNLVKNKCVNF |
| 14 | SARS-CoV-2 | NSNNLDSKVGGNYNYLYRLFRKSNLKPFERDISTEIYQAGSTPCNGVEGFNCYFPLQSYGFQ |
| | spike protein | PTNGVGYQPY |
| | RBM | |
| 15 | Human ACE2 | MSSSSWLLLSLVAVTAAQSTIEEQAKTFLDKFNHEAEDLFYQSSLASWNYNTNITEENVQN |
| | isoform 1 | MNNAGDKWSAFLKEQSTLAQMYPLQEIQNLTVKLQLQALQQNGSSVLSEDKSKRLNTILN |
| | | TMSTIYSTGKVCNPDNPQECLLLEPGLNEIMANSLDYNERLWAWESWRSEVGKQLRPLYEE |
| | | YVVLKNEMARANHYEDYGDYWRGDYEVNGVDGYDYSRGQLIEDVEHTFEEEKPLYEHLH |
| | | AYVRAKLMNAYPSYISPIGCLPAHLLGDMWGRFWTNLYSLTVPFGQKPNIDVTDAMVDQA |
| | | WDAQRIFKEAEKFFVSVGLPNMTQGFWENSMLTDPGNVQKAVCHPTAWDLGKGDFRILM |
| | | CTKVTMDDFLTAHHEMGHIQYDMAYAAQPFLLRNGANEGFHEAVGEIMSLSAATPKHLKS |
| | | IGLLSPDFQEDNETEINFLLKQALTIVGTLPFTYMLEKWRWMVFKGEIPKDQWMKKWWEM |
| | | KREIVGVVEPVPHDETYCDPASLFHVSNDYSFIRYYTRTLYQFQFQEALCQAAKHEGPLHKC |
| | | DISNSTEAGQKLFNMLRLGKSEPWTLALENVVGAKNMNVRPLLNYFEPLFTWLKDQNKNS |
| | | FVGWSTDWSPYADQSEKVRISLKSALGDKAYEWNDNEMYLFRSSVAYAMRQYFLKVKNQ |
| | | MILFGEEDVRVANLKPRISENFFVTAPKNVSDIIPRTEVEKAIRMSRSRINDAFRLNDNSLEFL |
| | | GIQPTLGPPNQPPVSIWLIVFGVVMGVIVVGIVILIFTGIRDRKKKNKARSGENPYASEDISKGE |
| | | NNPGFQNTDDVQTSF |
| 16 | Human ACE2 | MSSSSWLLLSLVAVTAAQSTIEEQAKTFLDKENHEAEDLEYQSSLASWNYNTNITEENVQN |
| | isoform 2 | MNNAGDKWSAFLKEQSTLAQMYPLQEIQNLTVKLQLQALQQNGSSVLSEDKSKRLNTILN |
| | | TMSTIYSTGKVCNPDNPQECLLLEPGLNEIMANSLDYNERLWAWESWRSEVGKQLRPLYEE |
| | | YVVLKNEMARANHYEDYGDYWRGDYEVNGVDGYDYSRGQLIEDVEHTFEEEKPLYEHLH |
| | | AYVRAKLMNAYPSYISPIGCLPAHLLGDMWGREWTNLYSLTVPFGQKPNIDVTDAMVDQA |
| | | WDAQRIFKEAEKFFVSVGLPNMTQGFWENSMLTDPGNVQKAVCHPTAWDLGKGDFRILM |
| | | CTKVTMDDFLTAHHEMGHIQYDMAYAAQPFLLRNGANEGFHEAVGEIMSLSAATPKHLKS |
| | | IGLLSPDFQEDNETEINFLLKQALTIVGTLPFTYMLEKWRWMVFKGEIPKDQWMKKWWEM |
| | | KREIVGVVEPVPHDETYCDPASLFHVSNDYSFIRYYTRTLYQFQFQEALCQAAKHEGPLHKC |
| | | DISNSTEAGQKLL |
| 17 | Human ACE2 | QSTIEEQAKTFLDKENHEAEDLEYQSSLASWNYNTNITEENVQNMNNAGDKWSAFLKEQST |
| | isoform 1 | LAQMYPLQEIQNLTVKLQLQALQQNGSSVLSEDKSKRLNTILNTMSTIYSTGKVCNPDNPQE |
| | extracellular | CLLLEPGLNEEMANSLDYNERLWAWESWRSEVGKQLRPLYEEYVVLKNEMARANHYEDY |
| | domain | GDYWRGDYEVNGVDGYDYSRGQLIEDVEHTFEEEKPLYEHLHAVVRAKLMNAYPSYISPIG |
| | | CLPAHLLGDMWGREWTNLYSLTVPFGQKPNEDVTDAMVDQAWDAQRIFKEAEKFFVSVGL |
| | | PNMTQGFVVENSMLTDPGNVQKAVCHPTAWDLGKGDFRILMCTKVTMDDFLTAHHEMGHI |
| | | QYDMAYAAQPFLLRNGANEGFHEAVGEEMSLSAATPKHLKSIGLLSPDFQEDNETEINFLLK |
| | | QALTIVGTLPFTYMLEKWRWMVFKGEIPKDQWMKKWWENIKREIVGVVEPVPHDETYCDP |
| | | ASLFHVSNDYSFIRYYTRTLYQFQFQEALCQAAKHEGPLHKCDISNSTEAGQKLFNMLRLG |
| | | KSEPWTLALENVVGAKNMNVRPLLNYFEPLFTWLKDQNKNSFVGWSTDWSPYADQSIKV |
| | | RISLKSALGDKAYEWNDNEMYLFRSSVAYAMRQYFLKVKNQMILFGEEDVRVANLKPRIS |
| | | FNFFVTAPKNVSDIIPRTEVEKAIRMSRSRINDAFRLNDNSLEFLGIQPTLGPPNQPPVS |
| 18 | Bat (Rhinolophus | MSGSSWELLSLVAVTAAQSTTEDLAKKELDDENSEAENLSHQSSLASWEYNTNISDENVQK |
| | ferrumequinum) | MDEAGAKWSDEYEKQSKLAKNESLEEHINDTVKLQLQILQQSGSPVLSEDKSKRLNSILNA |
| | ACE2 | MSTIYSTGKVCKPNNPQECLLLEPGLDNIMGTSKDYNERLWAWEGWRAEVGKQLRPLYEE |
| | | YVVLKNEMARGYHYEDYGDYWRRDYETEGSPDLEYSRDQLIKDVERIFAEIKPLYEQLHA |
| | | YVRTKLMDTYPFHISPTGCLPAHLLGDMWGREWTNLYPLTVPFGQKPNIDVTDAMLNQNVV |
| | | DAKRIFKEAEKFLVSIGLPNMTEGFWNNSMLTDPGDGRKVVCHPTAWDLGKGDFRIKMCT |
| | | KVTMEDFLTAHHEMGHIQYDMAYASQPYLLRNGANEGFHEAVGEVMSLSVATPEHLKTM |
| | | GLLSSDFLEDNETEINFLFKQALNIVGTLPLTYMLEKWRWMVFKGEIPKEEWMKKWWENIK |
| | | RKIVGVVEPVPHDETYCDPASLFHVANDYSFIRYYTRTIIEFQFHEALCRIAKHDGPLHKCGI |
| | | SNSTDAGEKLHQMLSVGKSQPWTSVLKDEVGSKNMDVGPLLRYFEPLYTVVLTEQNRKSEV |
| | | GWNTDWSPYADQSEKVWISLKSALGEKAYEWNNNEMYLFRSSVAYAMREYFLKTKNQTIL |
| | | FGEEDVWVSNLKPRISFNFYVTSPRNLSDIIPRPEVEGAIRMSRSRINDAFRLDDNSLEFLGIQ |
| | | PTLGPPYQPPVTIWLIVFGVVMAVVVVGIVVLIITGIRDRRKKDQARSEENPYSSVDLSKGEN |
| | | NPGFQNGNDVQTSF |
| 19 | Bat (Rhinolophus | QSTTEDLAKKELDDENSEAENLSHQSSLASWEYNTNISDENVQKMDEAGAKWSDFYEKQS |
| | ferrumequinum) | KLAKNESLEEHINDTVKLQLQILQQSGSPVLSEDKSKRLNSILNAMSTIYSTGKVCKPNNPQE |
| | ACE2 | CLLLEPGLDNIMGTSKDYNERLWAWEGWRAEVGKQLRPLYEEYVVLKNEMARGYHYED |
| | extracellular | YGDYWRRDYETEGSPDLEYSRDQLIKDVERIFAEIKPLYEQLHAYVRTKLMDTYPFHISPTG |
| | domain | CLPAHLLGDMWGRFWTNLYPLTVPFGQKPNEDVTDAMLNQNVVDAKRIFKEAEKFLVSIGL |
| | | PNMTEGFWNNSMLTDPGDGRKVVCHPTAWDLGKGDFREKMCTKVTMEDFLTAHHEMGHI |
| | | QYDMAYASQPYLLRNGANEGFHEAVGEVMSLSVATPEHLKTMGLLSSDFLEDNETEINFLF |
| | | KQALNIVGTLPLTYMLEKWRWMVFKGEIPKEEWMKKWVVEMKRKIVGVVEPVPHDETYC |
| | | DPASLFHVANDYSFIRYYTRTIFEFQFHEALCRIAKHDGPLHKCGISNSTDAGEKLHQMLSV |
| | | GKSQPWTSVLKDFVGSKNMDVGPLLRYFEPLYTWLTEQNRKSFVGWNTDWSPYADQSIKV |
| | | WISLKSALGEKAYEWNNNEMYLFRSSVAYAMREYFLKTKNQTTILFGEEDVWVSNLKPRISF |
| | | NFYVTSPRNLSDIIPRPEVEGAIRMSRSRINDAFRLDDNSLEFLGIQPTLGPPYQPPVT |
| 20 | Pangolin (Manis | MSGSSWLLLSLVAVTAAQSTSDEEAKTFLEKFNSEAEELSYQSSLASWNYNTNITDENVQK |
| | javanica) ACE2 | MNVAGAKWSTFYEEQSKIAKNYQLQNIQNDTIKRQLQALQLSGSSALSADKNQRLNTILNT |
| | | MSTIYSTGKVCNPGNPQECSLLEPGLDNIMESSKDYNERLWAWEGWRSEVGKQLRPLYEE |
| | | YVVLKNEMARANHYEDYGDYWRGDYEAEGANGYNYSRDHLIEDVEHIFTQIKPLYEHLH |
| | | AYVRAKLMDNYPSHISPTGCLPAHLLGDMWGRFWTNLYPLTVPFRQKPNEDVTDAMVNQT |
| | | WDANRIFKEAEKFFVSVGLPKMTQTFWENSMLTEPGDGRKVVCHPTAWDLGKHDFRIKM |
| | | CTKVTMDDFLTAHHEMGHIQYDMAYAMQPYLLRNGANEGFHEAVGELMSLSAATPKHLK |
| | | NIGLLPPDFYEDNETEINFLLKQALTIVGTLPFTYMLEKWRWMVFSGQIPKEQWMKKWWE |
| | | MKREIVGVVEPVPHDETYCDPASLFHVANDYSFIRYYTRTIYQFQFQEALCQTAKHEGPLHK |
| | | CDISNSAEAGQKLLQMLSLGKSKPWTLALERVVGTKNMDVRPLLNYFEPLLTWLKEQNKN |
| | | SFVGWNTDWSPYAAQSEKVRISLKSALGEKAYEWNDSEMYLFRSSVAYAMREYFSKVKKQ |
| | | TIPFEDECVRVSDLKPRVSFIFFVTLPKNVSAVIPRAEVEEAIRISRSRINDAFRLDDNSLEFLGI |
| | | QPTLQPPYQPPVTIWLIVFGVVMGVVVVGIVVLIFTGIRDRKKKDQARSEQNPYASVDLSKG |
| | | ENNPGFQNVDDVQTSF |
| 21 | Pangolin (Manis | QSTSDEEAKTFLEKFNSEAEELSYQSSLASWNYNTNITDENVQKMNVAGAKWSTFYEEQSK |
| | javanica) ACE2 | IAKNYQLQNIQNDTEKRQLQALQLSGSSALSADKNQRLNTILNTMSTIYSTGKVCNPGNPQE |
| | extracellular | CSLLEPGLDNIMESSKDYNERLWAWEGWRSEVGKQLRPLYEEYVVLKNEMARANHYEDY |
| | domain | GDYWRGDYEAEGANGYNYSRDHLIEDVEHIFTQIKPLYEHLHAYVRAKLMDNYPSHISPTG |
| | | CLPAHLLGDMWGRFWTNLYPLTVPFRQKPNEDVTDAMVNQTWDANRIFKEAEKFFVSVGL |
| | | PKMTQTFWENSMLTEPGDGRKVVCHPTAWDLGKHDFRIKMCTKVTMDDFLTAHHEMGHI |
| | | QYDMAYAMQPYLLRNGANEGFHEAVGEIMSLSAATPKHLKNIGLLPPDFYEDNETEINFLL |
| | | KQALTIVGTLPFTYMLEKWRWMVFSGQIPKEQWMKKWVVEMKREIVGVVEPVPHDETYCD |
| | | PASLFHVANDYSFIRYYTRTIYQFQFQEALCQTAKHEGPLHKCDISNSAEAGQKLLQMLSLG |
| | | KSKPWTLALERVVGTKNMDVRPLLNYFEPLLTWLKEQNKNSFVGWNTDWSPYAAQSIKV |
| | | RISLKSALGEKAYEWNDSEMYLFRSSVAYAMREYFSKVKKQTIPFEDECVRVSDLKPRVSFI |
| | | FFVTLPKNVSAVIPRAEVEEAIRISRSRINDAFRLDDNSLEFLGIQPTLQPPYQPPVT |
| 22 | Civet (Paguma | MSGSFWLLLSFAALTAAQSTTEELAKTFLETFNYEAQELSYQSSVASWNYNTNITDENAKN |
| | larvata) ACE2 | MNEAGAKWSAYYEEQSKLAQTYPLAEIQDAKEKRQLQALQQSGSSVLSADKSQRLNTILNA |
| | | MSTIYSTGKACNPNNPQECLLLEPGLDNIMENSKDYNERLWAWEGWRAEVGKQLRPLYEE |
| | | YVALKNEMARANNYEDYGDYWRGDYEEEWTGGYNYSRNQLIQDVEDTFEQEKPLYQHLH |
| | | AYVRAKLMDTYPSRISRTGCLPAHLLGDMWGRFWTNLYPLTVPFGQKPNEDVTDAMVNQN |
| | | WDARRIFKEAEKFFVSVGLPNMTQGFWENSMLTEPGDGRKVVCHPTAWDLGKGDFRIKM |
| | | CTKVTMDDFLTAHHEMGHIQYDMAYAAQPFLLRNGANEGFHEAVGEIMSLSAATPNHLKT |
| | | IGLLSPAFSEDNETEINFLLKQALTIVGTLPFTYMLEKWRWMVFKGAIPKEQWMQKWVVEM |
| | | KRNIVGVVEPVPHDETYCDPASLFHVANDYSFIRYYTRTIYQFQFQEALCQIAKHEGPLHKC |
| | | DISNSTEAGKKLLEMLSLGRSEPWTLALERVVGAKNMNVTPLLNYFEPLFTWLKEQNRNSF |
| | | VGWDTDWRPYSDQSEKVRISLKSALGEKAYEWNDNEMYLFRSSIAYAMREYFSKVKNQTIP |
| | | FVEDNVWVSDLKPRISENFFVTFSNNVSDVIPRSEVEDAIRMSRSRINDAFRLDDNSLEFLGIE |
| | | PTLSPPYRPPVTIWLIVFGVVMGAIVVGIVLLIVSGIRNRRKNDQAGSEENPYASVDLNKGEN |
| | | NPGFQHADDVQTSF |
| 23 | Civet (Paguma | QSTTEELAKTFLETFNYEAQELSYQSSVASWNYNTNITDENAKNMNEAGAKWSAYYEEQS |
| | larvata) ACE2 | KLAQTYPLAEIQDAKEKRQLQALQQSGSSVLSADKSQRLNTILNAMSTIYSTGKACNPNNPQ |
| | extracellular | ECLLLEPGLDNEMENSKDYNERLWAWEGWRAEVGKQLRPLYEEYVALKNEMARANNYED |
| | domain | YGDYWRGDYEEEWTGGYNYSRNQLIQDVEDTFEQEKPLYQHLHAYVRAKLMDTYPSRISR |
| | | TGCLPAHLLGDMWGREWTNLYPLTVPFGQKPNEDVTDAMVNQNVVDARRIFKEAEKFFVSV |
| | | GLPNMTQGFVVENSMLTEPGDGRKVVCHPTAWDLGKGDFRIKMCTKVTMDDFLTAHHEM |
| | | GHIQYDMAYAAQPFLLRNGANEGFHEAVGEEMSLSAATPNHLKTIGLLSPAFSEDNETEINF |
| | | LLKQALTIVGTLPFTYMLEKWRWMVFKGAIPKEQWMQKWVVEMKRNIVGVVEPVPHDET |
| | | YCDPASLFHVANDYSFIRYYTRTIYQFQFQEALCQIAKHEGPLHKCDISNSTEAGKKLLEML |
| | | SLGRSEPWTLALERVVGAKNMNVTPLLNYFEPLFTWLKEQNRNSFVGWDTDWRPYSDQSI |
| | | KVRISLKSALGEKAYEWNDNEMYLFRSSIAYAMREYFSKVKNQTIPFVEDNVWVSDLKPRI |
| | | SENFFVTFSNNVSDVIPRSEVEDAIRMSRSRINDAFRLDDNSLEFLGIEPTLSPPYRPPVT |
| 24 | Pig (Sus scrofa) | MSGSFWLLLSLIPVTAAQSTTEELAKTFLEKFNLEAEDLAYQSSLASWNYNTNITDENIQKM |
| | ACE2 | NDARAKWSAFYEEQSRIAKTYPLDEIQTLILKRQLQALQQSGTSGLSADKSKRLNTILNTMS |
| | | TIYSSGKVLDPNNPQECLVLEPGLDEIMENSKDYSRRLWAWESWRAEVGKQLRPLYEEYV |
| | | VLENEMARANNYEDYGDYWRGDYEVTGTGDYDYSRNQLMEDVERTFAEIKPLYEHLHAY |
| | | VRAKLMDAYPSRISPTGCLPAHLLGDMWGREVVTNLYPLTVPFGEKPSEDVTEAMVNQSWD |
| | | AIRIFEEAEKFFVSIGLPNMTQGFWNNSMLTEPGDGRKVVCHPTAWDLGKGDFREKMCTKV |
| | | TMDDFLTAHHEMGHIQYDMAYAIQPYLLRNGANEGFHEAVGEEMSLSAATPHYLKALGLL |
| | | PPDFYEDSETEINFLLKQALTIVGTLPFTYMLEKWRWMVFKGEIPKEQWMQKWVVEMKREI |
| | | VGVVEPLPHDETYCDPACLFHVAEDYSFIRYYTRTIYQFQFHEALCRTAKHEGPLYKCDISN |
| | | STEAGQKLLQMLSLGKSEPWTLALENIVGVKTMDVKPLLSYFEPLLTWLKAQNGNSSVGW |
| | | NTDWTPYADQSEKVRISLKSALGKEAYEWNDNEMYLFRSSIAYAMRNYFSSAKNETIPFGA |
| | | EDVWVSDLKPRISENFFVTSPANMSDIIPRSDVEKAISMSRSRINDAFRLDDNTLEFLGIQPTL |
| | | GPPDEPPVTVWLIIFGVVMGLVVVGIVVLIFTGIRDRRKKKQASSEENPYGSMDLSKGESNS |
| | | GFQNGDDIQTSF |
| 25 | Pig (Sus scrofa) | QSTTEELAKTFLEKFNLEAEDLAYQSSLASWNYNTNITDENIQKMNDARAKWSAFYEEQSR |
| | ACE2 | IAKTYPLDEIQTLILKRQLQALQQSGTSGLSADKSKRLNTILNTMSTIYSSGKVLDPNNPQEC |
| | extracellular | LVLEPGLDEEMENSKDYSRRLWAWESWRAEVGKQLRPLYEEYVVLENEMARANNYEDYG |
| | domain | DYWRGDYEVTGTGDYDYSRNQLMEDVERTFAEIKPLYEHLHAYVRAKLMDAYPSRISPTG |
| | | CLPAHLLGDMWGREWTNLYPLTVPFGEKPSIDVTEAMVNQSWDAIRIFEEAEKFFVSIGLPN |
| | | MTQGFVVNNSMLTEPGDGRKVVCHPTAWDLGKGDFREKMCTKVTMDDFLTAHHEMGHIQ |
| | | YDMAYAIQPYLLRNGANEGFHEAVGEMSLSAATPHYLKALGLLPPDFYEDSETEINFLLKQ |
| | | ALTIVGTLPFTYMLEKWRWMVFKGEIPKEQWMQKWWEMKREIVGVVEPLPHDETYCDPA |
| | | CLFHVAEDYSFIRYYTRTIYQFQFHEALCRTAKHEGPLYKCDISNSTEAGQKLLQMLSLGKS |
| | | EPWTLALENIVGVKTMDVKPLLSYFEPLLTWLKAQNGNSSVGWNTDWTPYADQSEKVRISL |
| | | KSALGKEAYEWNDNEMYLFRSSIAYAMRNYFSSAKNETIPFGAEDVWVSDLKPRISFNFFV |
| | | TSPANMSDIIPRSDVEKAISMSRSRINDAFRLDDNTLEFLGIQPTLGPPDEPPVT |
| 26 | SARS-CoV-2 | PNITNLCPFGEVFNATRFASVYAWNRKRISNCVADYSVLYNSASFSTFKCYGVSPTKLNDLC |
| | spike protein | FTNVYDSFVIRGDEVRQIAPGQTGKIADYNYKLPDDFTGCVIAWNSNNLDSKVGGNYNYLY |
| | RBD | RLFRKSNLKPFERDISTEIYQAGSTPCNGVEGFNCYFPLQSYGFQPTNGVGYQPYRVVVLSFE |
| | | LLHAPATVCGPKKS |
| 27 | SARS-CoV-2 | MLLVNQSHQGFNKEHTSKMVSAIVLYVLLAAAAHSAFAQCVNLTTRTQLPPAYTNSFTRG |
| | spike protein S1 | VYYPDKVFRSSVLHSTQDLFLPFFSNVTWFHAIHVSGTNGTKRFDNPVLPFNDGVYFASTEK |
| | subunit | SNIIRGWIFGTTLDSKTQSLLIVNNATNVVIKVCEFQFCNDPFLGVYYHKNNKSWMESEFRV |
| | | YSSANNCTFEYVSQPFLMDLEGKQGNFKNLREFVFKNEDGYFKIYSKHTPINLVRDLPQGFS |
| | | ALEPLVDLPIGINITRFQTLLALHRSYLTPGDSSSGWTAGAAAYYVGYLQPRTFLLKYNENG |
| | | TITDAVDCALDPLSETKCTLKSFTVEKGIYQTSNFRVQPTESIVRFPNITNLCPFGEVFNATRF |
| | | ASVYAWNRKRISNCVADYSVLYNSASFSTFKCYGVSPTKLNDLCFTNVYADSFVIRGDEVR |
| | | QIAPGQTGKIADYNYKLPDDFTGCVIAWNSNNLDSKVGGNYNYLYRLFRKSNLKPFERDIS |
| | | TEIYQAGSTPCNGVEGFNCYFPLQSYGFQPTNGVGYQPYRVVVLSFELLHAPATVCGPKKST |
| | | NLVKNKCVNFNFNGLTGTGVLTESNKKFLPFQQFGRDIADTTDAVRDPQTLEILDITPCSFG |
| | | GVSVITPGTNTSNQVAVLYQDVNCTEVPVAIHADQLTPTWRVYSTGSNVFQTRAGCLIGAE |
| | | HVNNSYECDIPIGAGICASYQTQTNSPRRAR |
| 28 | SARS-CoV spike | MNITNLCPFGEVFNATKFPSVYAWERKKISNCVADYSVLYNSTFFSTFKCYGVSATKLNDL |
| | protein RBD | CFSNVYADSFVVKGDDVRQIAPGQTGVIADYNYKLPDDFMGCVLAWNTRNIDATSTGNYN |
| | | YKYRYLRHGKLRPFERDISNVPFSPDGKPCTPPALNCYWPLNDYGFYTTTGIGYQPYRVVV |
| | | LSFELLNAPATYLSLNTAAAL |
| 29 | Human ACE2 | MSSSSWLLLSLVAVTAAQSTIEEQAKTFLDKFNHEAEDLFYQSSLASWNYNTNITEENVQN |
| | | MNNAGDKWSAFLKEQSTLAQMYPLQEIQNLTVKLQLQALQQNGSSVLSEDKSKRLNTILN |
| | | TMSTIYSTGKVCNPDNPQECLLLEPGLNEIMANSLDYNERLWAWESWRSEVGKQLRPLYEE |
| | | YVVLKNEMARANHYEDYGDYWRGDYEVNGVDGYDYSRGQLIEDVEHTFEEIKPLYEHLH |
| | | AYVRAKLMNAYPSYISPIGCLPAHLLGDMWGRFWTNLYSLTVPFGQKPNIDVTDAMVDQA |
| | | WDAQRIFKEAEKFFVSVGLPNMTQGFWENSMLTDPGNVQKAVCHPTAWDLGKGDFRILM |
| | | CTKVTMDDFLTAHHEMGHIQYDMAYAAQPFLLRNGANEGFHEAVGEIMSLSAATPKHLKS |
| | | IGLLSPDFQEDNETEINFLLKQALTIVGTLPFTYMLEKWRWMVFKGEIPKDQWMKKWWEM |
| | | KREIVGVVEPVPHDETYCDPASLFHVSNDYSFIRYYTRTLYQFQFQEALCQAAKHEGPLHKC |
| | | DISNSTEAGQKLFNMLRLGKSEPWTLALENVVGAKNMNVRPLLNYFEPLFTWLKDQNKNS |
| | | FVGWSTDWSPYADQSEKVRISLKSALGDRAYEWNDNEMYLFRSSVAYAMRQYFLKVKNQ |
| | | MILFGEEDVRVANLKPRISFNFFVTAPKNVSDIIPRTEVEKAIRMSRSRINDAFRLNDNSLEFL |
| | | GIQPTLGPPNQPPVSIWLIVFGVVMGVIVVGIVILIFTGIRDRKKKNKARSGENPYASEDISKGE |
| | | NNPGFQNTDDVQTSF |
| 30 | Human ACE2 | QSTIEEQAKTFLDKFNHEAEDLFYQSSLASWNYNTNITEENVQNMNNAGDKWSAFLKEQST |
| | extracellular | LAQMYPLQEIQNLTVKLQLQALQQNGSSVLSEDKSKRLNTILNTMSTIYSTGKVCNPDNPQE |
| | domain | CLLLEPGLNEBEANSLDYNERLWAWESWRSEVGKQLRPLYEEYVVLKNEMARANHYEDY |
| | | GDYWRGDYEVNGVDGYDYSRGQLIEDVEHTFEEEKPLYEHLHAVVRAKLMNAYPSYISPIG |
| | | CLPAHLLGDMWGRFWTNLYSLTVPFGQKPNEDVTDAMVDQAWDAQRIFKEAEKFFVSVGL |
| | | PNMTQGFVVENSMLTDPGNVQKAVCHPTAWDLGKGDFRILMCTKVTMDDFLTAHHEMGHI |
| | | QYDMAYAAQPFLLRNGANEGFHEAVGEBESLSAATPKHLKSIGLLSPDFQEDNETEINFLLK |
| | | QALTIVGTLPFTYMLEKWRWMVFKGEIPKDQWMKKWWENIKREIVGVVEPVPHDETYCDP |
| | | ASLFHVSNDYSFIRYYTRTLYQFQFQEALCQAAKHEGPLHKCDISNSTEAGQKLFNMLRLG |
| | | KSEPWTLALENVVGAKNMNVRPLLNYFEPLFTWLKDQNKNSFVGWSTDWSPYADQSIKV |
| | | RISLKSALGDRAYEWNDNEMYLFRSSVAYAMRQYFLKVKNQMILFGEEDVRVANLKPRISF |
| | | NFFVTAPKNVSDIIPRTEVEKAIRMSRSRINDAFRLNDNSLEFLGIQPTLGPPNQPPVS |
| 31 | Bat (Rhinolophus | MSSSSWLLLSLVAVTTAQFTTEDLAKIFLDEFNSEAENLSYQSSLASWDYNTNINDENVQK |
| | sinicus) ACE2 | MDEAGAKWSAFYEEQSKLAKNYPLEQIQNVTVKLQLQILQQSGSPVLSEDKSKRLNSILNA |
| | | MSTIYSTGKVCKPNKPHECLLLEPGLDNIMGTSKDYSERLWAWEGWRAEVGKQLRPLYEE |
| | | YVVLKNEMARGYHYEDYGDYWRRDYETEESPGPGYSRDQLMKDVERIFTEEKPLYEHLHA |
| | | YVRAKLMDTYPFHISPTGCLPAHLLGDMWGRFWTNLYPLTVPFGQKPNIDVTDEMLKQGW |
| | | DADRIFKEAEKFFVSVGLPNMTEGFWNNSMLTEPGDGRKVVCHPTAWDLGKGDFREKMCT |
| | | KVTMEDFLTAHHEMGHIQYDMAYASQPYLLRNGANEGFHEAVGEVMSLSVATPKHLKTM |
| | | GLLSPDFREDNETEINFLLKQALNIVGTLPFTYMLEKWRWMVFKGEIPKEEWMKKWVVEMK |
| | | RKIVGVVEPVPHDETYCDPASLFHVANDYSFIRYYTRTIIEFQFHEALCRIAQHDGPLHKCDI |
| | | SNSTDAGKKLHQMLSVGKSQAWTKTLEDIVDSRNMDVGPLLRYFEPLYTWLQEQNRKSYV |
| | | GWNTDWSPYSDQSEKVRISLKSALGENAYEWNDNEMYLFRSSVAYAMREYFLKEKHQTIL |
| | | FGAENVWVSNLKPRISFNFHVTSPGNLSDIIPRPEVEGAIRMSRSRINDAFRLDDNSLEFLGIQ |
| | | PTLGPPYQPPVTIWLIVFGVVMAVVVVGIVVLIITGIRDRRKTDQARSEENPYSSVDLSKGEN |
| | | NPGFQNGDDVQTSF |
| 32 | Bat (Rhinolophus | QFTTEDLAKIFLDEFNSEAENLSYQSSLASWDYNTNINDENVQKMDEAGAKWSAFYEEQSK |
| | sinicus) ACE2 | LAKNYPLEQIQNVTVKLQLQILQQSGSPVLSEDKSKRLNSILNAMSTIYSTGKVCKPNKPHE |
| | extracellular | CLLLEPGLDNBEGTSKDYSERLWAWEGWRAEVGKQLRPLYEEYVVLKNEMARGYHYEDY |
| | domain | GDYWRRDYETEESPGPGYSRDQLMKDVERIFTEEKPLYEHLHAVVRAKLMDTYPFHISPTG |
| | | CLPAHLLGDMWGRFWTNLYPLTVPFGQKPNEDVTDEMLKQGWDADRIFKEAEKFFVSVGL |
| | | PNMTEGFWNNSMLTEPGDGRKVVCHPTAWDLGKGDFREKMCTKVTMEDFLTAHHEMGHI |
| | | QYDMAYASQPYLLRNGANEGFHEAVGEVMSLSVATPKHLKTMGLLSPDFREDNETEINFLL |
| | | KQALNIVGTLPFTYMLEKWRWMVFKGEIPKEEWMKKWVVEMKRKIVGVVEPVPHDETYC |
| | | DPASLFHVANDYSFIRYYTRTIFEFQFHEALCRIAQHDGPLHKCDISNSTDAGKKLHQMLSV |
| | | GKSQAWTKTLEDIVDSRNMDVGPLLRYFEPLYTWLQEQNRKSYVGWNTDWSPYSDQSIKV |
| | | RISLKSALGENAYEWNDNEMYLFRSSVAYAMREYFLKEKHQTILFGAENVWVSNLKPRISF |
| | | NFHVTSPGNLSDIIPRPEVEGAIRMSRSRINDAFRLDDNSLEFLGIQPTLGPPYQPPVT |
| 33 | Pig (Sus scrofa) | MSGSFWLLLSLIPVTAAQSTTEELAKTFLEKFNLEAEDLAYQSSLASWTINTNITDENIQKMN |
| | ACE2 | DARAKWSAFYEEQSRIAKTYPLDEIQTLILKRQLQALQQSGTSGLSADKSKRLNTILNTMSTI |
| | | YSSGKVLDPNNPQECLVLEPGLDEBEENSKDYSRRLWAWESWRAEVGKQLRPLYEEYVVL |
| | | ENEMARANNYEDYGDYWRGDYEVTGTGDYDYSRNQLMEDVERTFAEIKPLYEHLHAVVR |
| | | AKLMDAYPSRISPTGCLPAHLLGDMWGREWTNLYPLTVPFGEKPSEDVTEAMVNQSWDAIR |
| | | IFEEAEKFFVSIGLPNMTQGFWNNSMLTEPGDGRKVVCHPTAWDLGKGDFREKMCTKVTM |
| | | DDFLTAHHEMGHIQYDMAYAIQPYLLRNGANEGFHEAVGEEMSLSAATPHYLKALGLLPPD |
| | | FYEDSETEINFLLKQALTIVGTLPFTYMLEKWRWMVFKGEIPKEQWMQKWVVEMKREIVGV |
| | | VEPLPHDETYCDPACLFHVAEDYSFIRYYTRTIYQFQFHEALCRTAKHEGPLYKCDISNSTEA |
| | | GQKLLQMLSLGKSEPWTLALENIVGVKTMDVKPLLSYFEPLLTVVLKAQNGNSSVGWNTD |
| | | WTPYADQSEKVRISLKSALGEDAYEWNDNEMYLFRSSIAYAMRNYFSSAKNETIPFGAVDV |
| | | WVSDLKPRISENFFVTSPANMSDIIPRSDVEKAISMSRSRINDAFRLDDNTLEFLGIQPTLGPP |
| | | DEPPVTVWLIEFGVVMGLVVVGIVVLIFTGIRDRRKKKQASSEENPYGSMDLSKGESNSGFQ |
| | | NGDDIQTSF |
| 34 | Pig (Sus scrofa) | QSTTEELAKTFLEKFNLEAEDLAYQSSLASWTINTNITDENIQKMNDARAKWSAFYEEQSRI |
| | ACE2 | AKTYPLDEIQTLILKRQLQALQQSGTSGLSADKSKRLNTILNTMSTIYSSGKVLDPNNPQECL |
| | extracellular | VLEPGLDEEMENSKDYSRRLWAWESWRAEVGKQLRPLYEEYVVLENEMARANNYEDYGD |
| | domain | YWRGDYEVTGTGDYDYSRNQLMEDVERTFAEIKPLYEHLHAVVRAKLMDAYPSRISPTGC |
| | | LPAHLLGDMWGREWTNLYPLTVPFGEKPSIDVTEAMVNQSWDAIRIFEEAEKFFVSIGLPN |
| | | MTQGFVVNNSMLTEPGDGRKVVCHPTAWDLGKGDFREKMCTKVTMDDFLTAHHEMGHIQ |
| | | YDMAYAIQPYLLRNGANEGFHEAVGEMSLSAATPHYLKALGLLPPDFYEDSETEINFLLKQ |
| | | ALTIVGTLPFTYMLEKWRWMVFKGEIPKEQWMQKWVVEMKREIVGVVEPLPHDETYCDPA |
| | | CLFHVAEDYSFIRYYTRTIYQFQFHEALCRTAKHEGPLYKCDISNSTEAGQKLLQMLSLGKS |
| | | EPWTLALENIVGVKTMDVKPLLSYFEPLLTWLKAQNGNSSVGWNTDWTPYADQSEKVRISL |
| | | KSALGEDAYEWNDNEMYLFRSSIAYAMRNYFSSAKNETIPFGAVDVWVSDLKPRISENFEV |
| | | TSPANMSDHPRSDVEKAISMSRSRINDAFRLDDNTLEFLGIQPTLGPPDEPPVT |

Having thus described in detail preferred embodiments of the present disclosure, it is to be understood that the disclosure defined by the above paragraphs is not to be limited to particular details set forth in the above description as many apparent variations thereof are possible without departing from the spirit or scope of the present disclosure.

### REFERENCES

1. Wang, C., Horby, P. W., Hayden, F. G. & Gao, G. F. A novel coronavirus outbreak of global health concern. Lancet 395, 470-473, doi:10.1016/S0140-6736(20)30185-9 (2020).
2. WHO. COVID-19 Status Report. https://www.who.int/emergencies/diseases/novel-coronavirus-2019/situation-reports. Accessed on 18 Apr. 2020 (2020).
3. Zhou, P. et al. A pneumonia outbreak associated with a new coronavirus of probable bat origin. Nature 579, 270-273, doi:10.1038/s41586-020-2012-7 (2020).
4. Coronaviridae Study Group of the International Committee on Taxonomy of, V. The species Severe acute respiratory syndrome-related coronavirus: classifying 2019-nCoV and naming it SARS-CoV-2. Nat Microbiol 5, 536-544, doi:10.1038/s41564-020-0695-z (2020).
5. Peiris, J. S. et al. Coronavirus as a possible cause of severe acute respiratory syndrome. Lancet 361, 1319-1325 (2003).
6. Petherick, A. Developing antibody tests for SARS-CoV-2. Lancet 395, 1101-1102, doi:10.1016/50140-6736(20)30788-1 (2020).
7. Hoffmann, M. et al. SARS-CoV-2 Cell Entry Depends on ACE2 and TMPRSS2 and Is Blocked by a Clinically Proven Protease Inhibitor. Cell 181, 271-280 e278, doi:10.1016/j.cell.2020.02.052 (2020).
8. Nie, J. et al. Establishment and validation of a pseudovirus neutralization assay for SARS-CoV-2. Emerging microbes & infections 9, 680-686, doi:10.1080/22221751.2020.1743767 (2020).
9. Tang, Y. W., Schmitz, J. E., Persing, D. H. & Stratton, C. W. The Laboratory Diagnosis of COVID-19 Infection: Current Issues and Challenges. J Clin Microbiol, doi:10.1128/JCM.00512-20 (2020).
10. Okba, N. M. A. et al. Severe Acute Respiratory Syndrome Coronavirus 2-Specific Antibody Responses in Coronavirus Disease 2019 Patients. Emerg Infect Dis 26, doi:10.3201/eid2607.200841 (2020).
11. Stadlbauer, D. et al. SARS-CoV-2 Seroconversion in Humans: A Detailed Protocol for a Serological Assay, Antigen Production, and Test Setup. Curr Protoc Microbiol 57, e100, doi:10.1002/cpmc.100 (2020).
12. Li, W. et al. Angiotensin-converting enzyme 2 is a functional receptor for the SARS coronavirus. Nature 426, 450-454 (2003).
13. Ou, X. et al. Characterization of spike glycoprotein of SARS-CoV-2 on virus entry and its immune cross-reactivity with SARS-CoV. Nat Commun 11, 1620, doi:10.1038/s41467-020-15562-9 (2020).
14. BBC. Coronavirus: Double warning over antibody tests. https://www.bbc.com/news/uk-52335210 Accessed on 18-04-2020 (2020).
15. Burton, D. R. Antibodies, viruses and vaccines. Nat Rev Immunol 2, 706-713, doi:10.1038/nri891 (2002).
16. Klasse, P. J. Neutralization of Virus Infectivity by Antibodies: Old Problems in New Perspectives. Adv Biol 2014, doi:10.1155/2014/157895 (2014).
17. Li, W. et al. Bats are natural reservoirs of SARS-like coronaviruses. Science 310, 676-679 (2005).
18. Young, P. L. et al. Serologic evidence for the presence in Pteropus bats of a paramyxovirus related to equine morbillivirus. Emerg Infect Dis 2, 239-240 (1996).
19. Yob, J. M. et al. Nipah virus infection in bats (order Chiroptera) in peninsular Malaysia. Emerg Infect Dis 7, 439-441. (2001).
20. Smith, I. et al. Identifying Hendra virus diversity in pteropid bats. PLoS ONE 6, e25275, doi:10.1371/journal.pone.0025275 PONE-D-11-12458 [pii] (2011).
21. Barr, J. et al. Isolation of multiple novel paramyxoviruses from pteropid bat urine. J Gen Virol 96, 24-29, doi:10.1099/vir.0.068106-0 (2015).
22. Leroy, E. M. et al. Fruit bats as reservoirs of Ebola virus. Nature 438, 575-576 (2005).
23. Zwick, M. B. et al. Neutralization synergy of human immunodeficiency virus type 1 primary isolates by cocktails of broadly neutralizing antibodies. J Virol 75, 12198-12208 (2001).
24. Howell, K. A. et al. Cooperativity Enables Non-neutralizing Antibodies to Neutralize Ebolavirus. Cell Rep 19, 413-424, doi:10.1016/j.celrep.2017.03.049 (2017).
25. Besselaar, T. G. & Blackburn, N. K. The synergistic neutralization of Rift Valley fever virus by monoclonal antibodies to the envelope glycoproteins. Arch Virol 125, 239-250, doi:10.1007/bf01309641 (1992).
26. Young, B. E. et al. Epidemiologic Features and Clinical Course of Patients Infected With SARS-CoV-2 in Singapore. JAMA, doi:10.1001/jama.2020.3204 (2020).
27. Jiang, L. et al. Detection of viral respiratory pathogens in mild and severe acute respiratory infections in Singapore. Sci Rep 7, 42963, doi:10.1038/srep42963 (2017).
28. Yu, M. et al. Determination and application of immunodominant regions of SARS coronavirus spike and nucleocapsid proteins recognized by sera from different animal species. Journal of Immunological Methods 331, 1-12 (2008).
29. Crameri, G. et al. Experimental Infection and Response to Rechallenge of Alpacas with Middle East Respiratory Syndrome Coronavirus. Emerg Infect Dis 22, 1071-1074, doi:10.3201/eid2206.160007 (2016).
30. Normile D. Singapore claims first use of antibody test to track coronavirus infections. Science 2020, doi:10.1126/science.abb4942
31. Amanat F, Nguyen T H O, Chromikova V et al. A serological assay to detect SARS-CoV-2 seroconversion in humans. medRxiv 2020 https://doi.org/10.1101/2020.03.17.20037713.
32. Okba N M A, Muller Mass., Li W et al. SARS-CoV-2 specific antibody responses in COVID-19 patients. medRxiv 2020 medRxiv https://doi.org/10.1101/2020.03.18.20038059.
33. Schuh A J, Amman B R, Sealy T K et al. Antibody-Mediated Virus Neutralization Is Not a Universal Mechanism of Marburg, Ebola, or Sosuga Virus Clearance in Egyptian Rousette Bats. J Infect Dis 2019 219(11):1716-1721.
34. Li F, Li W, Farzan M, Harrison S. C. Structure of SARS coronavirus spike receptor-binding domain complexed with receptor. Science 2005 309(5742):1864-8. S
35. Zhu Z, Chakraborti S, He Y et al. Potent cross-reactive neutralization of SARS coronavirus isolates by human monoclonal antibodies. Proc Natl Acad Sci USA 2007 104(29):12123-8.
36. Bossart K N, McEacherna J A, Hickey A C et al. Neutralization assays for differential henipavirus serology using Bio-Plex Protein Array Systems. J Viol Meth 2007 142: 29-40.
37. Lam T T-Y, Sum M H-H, Zhu H-C et al. Identification of 2019-nCoV related coronaviruses in Malayan pangolins in southern China. bioRxiv 2020 https://doi.org/10.1101/2020.02.13.945485.
38. Wang N, Shi X, Jiang L et al. Structure of MERS-CoV spike receptor-binding domain complexed with human receptor DPP4. Cell Res 2013 23(8):986-93.

What is claimed is:

1. A method of analysing a sample for the presence of antibodies to a SARSr-CoV, comprising:
contacting the sample with: (i) a spike protein of a severe acute respiratory syndrome-related coronavirus (SARSr-CoV), a S1 subunit or a receptor binding domain (RBD) of a SARSr-CoV spike protein, wherein the spike protein or the S1 subunit comprises the RBD of the SARSr-CoV spike protein, and (ii) an ACE2 protein or an extracellular domain of an ACE2 protein or a fragment thereof which binds specifically to the spike protein, the S1 subunit or the RBD of (i), and
determining the level of interaction between the spike protein, the S1 subunit or the RBD of (i) and the ACE2 protein or the ACE2 protein extracellular domain of (ii),
wherein the spike protein, the S1 subunit or the RBD of (i) or ACE2 protein or the ACE2 protein extracellular domain of (ii) is conjugated to a) a detection entity, or b) a biotin which binds an avidin or streptavidin-conjugated detection entity, for determining the interaction between (i) and (ii); and
(a) the spike protein, the S1 subunit or the RBD of (i) is conjugated to the detection entity or the biotin, and wherein the ACE2 protein or the ACE2 protein extracellular domain of (ii) is immobilised on a solid support; or
(b) the ACE2 protein or the ACE2 protein extracellular domain of (ii) is conjugated to the detection entity or the biotin, and wherein spike protein, the S1 subunit or the RBD of (i) is immobilised on a solid support.

2. The method according to claim 1, wherein the sample is a blood sample, a lymph sample, a saliva sample, or a synovial fluid sample.

3. The method according to claim 1, wherein the SARSr-CoV is SARS-CoV-2 or a variant thereof.

4. The method according to claim 1, wherein the spike protein, the S1 subunit or the RBD of (i) is conjugated to the detection entity or the biotin, and wherein the ACE2 protein or the ACE2 protein extracellular domain of (ii) is immobilised on a solid support.

5. The method according to claim 1, wherein the ACE2 protein or the ACE2 protein extracellular domain of (ii) is conjugated to the detection entity or the biotin, and wherein the spike protein, the S1 subunit or the RBD of (i) is immobilised on a solid support.

6. The method according to claim 1, wherein the detection entity is a horseradish peroxidase, an alkaline phosphatase, an acridinium compound, a phycoerythrin, or a fluorescein isothiocyanate.

7. The method according to claim 1, wherein the detection entity is a horseradish peroxidase.

8. The method according to claim 1, wherein the spike protein or the S1 subunit of (i) comprises the S1 subunit.

9. The method according to claim 1, wherein the S1 subunit comprises an amino acid sequence having SEQ ID NO: 12 or 27.

10. The method according to claim 1, wherein the RBD comprises an amino acid sequence having SEQ ID NO: 13 or 26.

11. The method according to claim 1, wherein the extracellular domain of ACE2 protein comprises an amino acid sequence having SEQ ID NO: 17 or 30.

12. The method according to claim 1, wherein the spike protein, the S1 subunit or the RBD of (i) is at a quantity that is:
(a) sufficient to produce a detectable signal of interaction between the spike protein, the S1 subunit or the RBD of (i) and the ACE2 protein or the ACE2 protein extracellular domain of (ii) in the absence of the sample, and/or
(b) less than or equal to, in molar ratio, the quantity of the antibodies to the SARSr-CoV in the sample, and/or
(c) a minimal quantity required to produce a detectable signal of interaction between the spike protein, the S1 subunit or the RBD of (i) and the ACE2 protein or the ACE2 protein extracellular domain of (ii) in the absence of the sample.

13. The method according to claim 1, wherein the solid support is a microtiter plate or a bead.

14. The method according to claim 13, wherein the detection entity is a horseradish peroxidase, an alkaline phosphatase, an acridinium compound, a phycoerythrin, or a fluorescein isothiocyanate.

15. The method according to claim 13, wherein the quantity of the spike protein, the S1 subunit or the RBD of (i) immobilized on the solid support is regulated by adjusting the quantity of the spike protein, the S1 subunit or the RBD of (i) incubated with the solid support.

16. The method according to claim 1, comprising:
contacting the sample with the spike protein, the S1 subunit or the RBD of (i),
immobilizing the spike protein, the S1 subunit or the RBD of (i) to the solid support,
contacting the spike protein, the S1 subunit or the RBD of (i) immobilized on the solid support with the ACE2 protein or the ACE2 protein extracellular domain of (ii), and
determining the level of interaction between the spike protein, the S1 subunit or the RBD of (i) and the ACE2 protein or the ACE2 protein extracellular domain of (ii),
wherein the ACE2 protein or the ACE2 protein extracellular domain of (ii) is conjugated to the detection entity, and
(a) the spike protein, the S1 subunit or the RBD of (i) is conjugated to a biotin, the solid support is a microtiter plate or a bead, and the solid support is conjugated with an avidin or a streptavidin, or
(b) the spike protein, the S1 subunit or the RBD of (i) is conjugated to an avidin or a streptavidin, the solid support is a bead, and the solid support is conjugated with a biotin.

17. The method according to claim 1, comprising:
immobilizing the spike protein, the S1 subunit or the RBD of (i) to the solid support,
contacting the sample with the spike protein, the S1 subunit or the RBD of (i) immobilized on the solid support,
contacting the spike protein, the S1 subunit or the RBD of (i) immobilized on the solid support with the ACE2 protein or the ACE2 protein extracellular domain of (ii), and
determining the level of interaction between the spike protein, the S1 subunit or the RBD of (i) and the ACE2 protein or the ACE2 protein extracellular domain of (ii),
wherein the ACE2 protein or the ACE2 protein extracellular domain of (ii) is conjugated to a) the detection entity, or b) the biotin which binds an avidin or streptavidin-conjugated detection entity.

18. The method according to claim 17, wherein the solid support is a microtiter plate or a bead.

19. The method according to claim 18, wherein the solid support is a bead, and the quantity of the spike protein, the S1 subunit or the RBD of (i) immobilized on the bead is regulated by adjusting a) the quantity of the spike protein, the S1 subunit or the RBD of (i) incubated with the bead, b) the quantity of the bead incubated with the spike protein, the S1 subunit or the RBD of (i), and/or c) the surface area of a bead for conjugating the spike protein, the S1 subunit or the RBD of (i).

20. The method according to claim 18, wherein the solid support is a bead, and the molar ratio of the spike protein, the S1 subunit or the RBD of (i) to the antibodies to a SARSr-CoV in the sample is optimized by adjusting the spike protein, the S1 subunit or the RBD of (i) immobilized on the bead.

21. A method of determining whether a subject is or has been infected with a SARSr-CoV, comprising:
contacting a sample obtained from the subject with: (i) a spike protein of a SARSr-CoV, a S1 subunit or a RBD of SARSr-CoV spike protein, wherein the spike protein or the S1 subunit comprises the RBD of the SARSr-CoV spike protein, and (ii) an ACE2 protein or an extracellular domain of an ACE2 protein or a fragment thereof which binds specifically to the spike protein, the S1 subunit or the RBD of (i), and
determining the level of interaction between the spike protein, the S1 subunit or the RBD of (i) and the ACE2 protein or the ACE2 protein extracellular domain of (ii),
wherein the spike protein, the S1 subunit or the RBD of (i) or ACE2 protein or the ACE2 protein extracellular domain of (ii) is conjugated to a) a detection entity or b) a biotin which binds an avidin or streptavidin-conjugated detection entity, for determining the interaction between (i) and (ii); and
(a) the spike protein, the S1 subunit or the RBD of (i) is conjugated to the detection entity or the biotin, and wherein the ACE2 protein or the ACE2 protein extracellular domain of (ii) is immobilised on a solid support; or
(b) the ACE2 protein or the ACE2 protein extracellular domain of (ii) is conjugated to the detection entity or the biotin, and wherein spike protein, the S1 subunit or the RBD of (i) is immobilised on a solid support.

22. The method according to claim 21, wherein the spike protein, the S1 subunit or the RBD of (i) is conjugated to the detection entity or the biotin, and wherein the ACE2 protein or the ACE2 protein extracellular domain of (ii) is immobilised on a solid support.

23. The method according to claim 21, wherein the detection entity is a horseradish peroxidase, an alkaline phosphatase, an acridinium compound, a phycoerythrin, or a fluorescein isothiocyanate.

24. The method according to claim 21, wherein the RBD comprises an amino acid sequence having SEQ ID NO: 13 or 26.

25. The method according to claim 21, wherein the SARSr-CoV is SARS-CoV-2 or a variant thereof.

26. The method according to claim 21, wherein the solid support is a microtiter plate or a bead.
